(19) United States
(12) Patent Application Publication
(10) Pub. No.: US 2026/0188518 A1
(43) Pub. Date: Jul. 2, 2026

(54) DETECTION OF RISK OF PRE-ECLAMPSIA IN OBESE PREGNANT WOMEN

(71) Applicant: METABOLOMIC DIAGNOSTICS LIMITED, Little Island Co. Cork (IE)

(72) Inventors: Robin TUYTTEN, Cork (IE); Gregoire THOMAS, Lokeren (BE)

(73) Assignee: METABOLOMIC DIAGNOSTICS LIMITED, Little Island Co. Cork (IE)

(21) Appl. No.: 19/552,021

(22) Filed: Feb. 27, 2026

Related U.S. Application Data
(62) Division of application No. 17/387,528, filed on Jul. 28, 2021.
Application No. 17/387,528 is a continuation of application No. PCT/EP2020/051959, filed on Jan. 27, 2020.

(30) Foreign Application Priority Data
Jan. 29, 2019 (EP) 19154278.6

(51) Int. Cl. G16H 50/30

(52) U.S. Cl. CPC G16H 50/30

(57) ABSTRACT

A computer implemented method of early prediction of risk of pre-eclampsia in a pregnant obese woman is described. The method comprises the steps of inputting abundance values for a panel of obese pregnancy specific metabolite biomarkers obtained from an assayed biological sample into a computational model, in which the biological sample is obtained from an obese pregnant woman at 8 to 24 weeks of pregnancy, and inputting a patient parameter for the pregnant obese woman into the computational model selected from at least one of ethnicity, risk of gestational diabetes, fetal sex, number of pregnancies and level of obesity. The computational model is configured to select a subset comprising at least two of the obese pregnancy specific metabolite biomarkers based on the patient parameter input, correlate abundance values for the subset of obese pregnancy specific metabolite biomarkers with risk of pre-eclampsia, and output a predicted risk of pre-eclampsia for the pregnant obese woman.

### CROSS-REFERENCE TO RELATED APPLICATIONS

This application is a divisional under 35 U.S.C. § 121 of co-pending U.S. application Ser. No. 17/387,528 filed Jul. 28, 2021, which is a continuation under 35 U.S.C. § 120 of co-pending International Application No. PCT/EP2020/051959 filed Jan. 27, 2020, which designates the U.S. and claims benefit under 35 U.S.C. § 119 (a) of EP Provisional application Ser. No. 19/154,278.6 filed Jan. 29, 2019, the contents of which are incorporated herein by reference in their entireties.

### FIELD OF THE INVENTION

The present invention relates to method of assessing the risk of an obese pregnant woman developing pre-eclampsia at an early stage in pregnancy.

### BACKGROUND TO THE INVENTION

Pre-Eclampsia (PE) is a disorder specific to pregnancy which occurs in 2-8% of all pregnancies. PE originates in the placenta and manifests as new-onset hypertension and proteinuria after 20 weeks' gestation. PE remains a leading cause of maternal and perinatal morbidity and mortality: each year 70,000 mothers and 500,000 infants die from the direct consequences of PE. Maternal complications of PE include cerebrovascular accidents, liver rupture, pulmonary oedema or acute renal failure. For the fetus, placental insufficiency causes fetal growth restriction, which is associated with increased neonatal morbidity and mortality. To date, the only cure for PE is delivery of the placenta, and hence the baby. Consequently, iatrogenic prematurity adds to the burden of neonatal morbidity and mortality. The impact of PE on the health of patients is not restricted to the perinatal period: affected mothers have a lifelong increased risk of cardiovascular disease, stroke and type 2 diabetes mellitus. Children born prematurely as a result of PE may have neurocognitive development issues ranging from mild learning difficulties to severe disabilities. In the longer term young children and adolescents of pregnancies complicated by PE exhibit increased blood pressure and BMI compared to their peers, with increased incidences of diabetes, obesity, hypertension and cardiac disease.

Obesity increases the overall risk of pre-eclampsia by approximately 2— to 3-fold. The risk of pre-eclampsia progressively increases with increasing BMI. The increase in risk with obesity is observed across different ethnicities (and references therein). Within the context of the global obesity epidemic, it is not outlandish to expect that pre-eclampsia incidence will increase over time; it is expected that by 2025, one in every five women of reproductive age worldwide will have obesity of whom at least 7-9% are likely to develop pre-eclampsia. This trend might already be apparent in the US, with a 25% increase in pre-eclampsia rate reported over the period 1987-2004.

Whilst there are currently no ready available treatments to cure pre-eclampsia when it manifests, there are some drug treatments, i.e., aspirin, metformin and others, which have the potential to prevent some of the pre-eclampsia cases developing. Interestingly, the effectiveness of these treatments might associate with a specific form of pre-eclampsia or a specific pregnancy sub-population. In this context Aspirin has been confirmed to effectively reduce the occurrence of a form of pre-eclampsia which is characterised by placental compromise and which is associated with an early manifestation of pre-eclampsia, i.e., preterm pre-eclampsia. Differently, the treatment with Metformin has been focused on treating obese pregnant women.

However, for these prophylactic interventions with therapeutics to impact on the incidence of pre-eclampsia at the population level, health care providers need to have a stratification tool, or test, which combines the following two attributes: identification of these pregnancies at increased risk for the disease and triaging the pregnancies to the appropriate treatment. These requirements follow the precautionary principle that one should not do harm to the pregnant woman and her unborn child; a blanket administration of drugs to all pregnancies in order to prevent pre-eclampsia in some, might incur unnecessary health risks (e.g., due to treatment side effects) in these who are not at increased risk in the first place.

In the case of identifying of pregnancies at increased risk of early onset and/or preterm pre-eclampsia, attempts are being made to deliver on a combination of a risk stratification step, which stratifies up to 10% of the pregnancy population into a high risk group at the end of first trimester/beginning of the 2nd trimester, and then to prophylactically treat this at-risk population with aspirin with the aim to prevent some of the early onset and/or preterm pre-eclampsia cases. In this case, the risk algorithm combines maternal history (fi, history of pre-eclampsia) and characteristics (fi, race, Body Mass Index (bmi)), biophysical findings (fi, blood pressure readings and Ultrasound measurements indicative for compromised placental perfusion) and biochemical factors (pregnancy-associated plasma protein A (gene: PAPPA) and Placental Growth Factor (Gene: PGF)).

Whilst there is progress made to identify these women who may benefit from a treatment with aspirin to prevent a fraction of preterm pre-eclampsia (a 62% reduction in incidence is reported), the applicants realized there is not a viable risk stratification test to identify-within the obese pregnant population-those pregnant women who can benefit from a prophylactic treatment such as, for example, metformin. It is of note that the reduction in pre-eclampsia incidence associated with Metformin is reported to be 75%.

The availability of a test to identify those at risk for pre-eclampsia within the obese pregnant population will also allow for testing the effectiveness of interventions other than metformin to prevent pre-eclampsia in this specific patient population, including but not restricted to;
Aspirin;
Low Molecular Weight Heparin;
Restriction of weight gain by either caloric intake reduction or life style changes;
Interventions to lower the glycemic index, including but not restricted to, insulin, glycemic index lowering probiotics; Citrulline
Antioxidants, including but not limited to, antioxidant vitamins (e.g., ascorbic acid, -tocopherol, -carotene), inorganic antioxidants (e.g., selenium), and a plant-derived polyphenols,
antioxidants to mitochondria, including but not limited to, Mito VitE and ergothioneine;
statins, including but not limited to, Pravastin.
Therapies involving the use of anti-inflammatory or immunosuppressive agents like, but not limited to, tacrolimus, or sulfalazine.
In addition, one can easily envision preferred therapeutic combinations like, but not limited to, aspirin and metformin; or metformin and sulfalazine.

Vieira et al explored the development of a prediction model for pre-eclampsia in the obese within the SCOPE study, whereby factors for a multivariable model were selected from the extensive collection of patient metadata collected in SCOPE, and a wide array of protein markers as quantified within the SCOPE study. For reference and benchmark reasons, the results of Vieira et al are quoted here: Factors associated with pre-eclampsia in univariable analysis (p<=0.05) were family history of thrombotic disease (p (<0.001); family history of pre-eclampsia or gestational hypertension (p=0.02); HDL cholesterol (MoM) (p=0.03); adiponectin (p=0.03); natriuretic peptide A (ANP) propeptide (p=0.05); natriuretic peptide B (MoM) (p=0.03); placental growth factor (PIGF) (p<0.001) and abnormal uterine artery Doppler (UtA-RI) (p=0.004). In multivariable analysis family history thrombotic disease (OR 2.48; 95% CI 1.38-4.45); PIGF (per log unit; inverted data-effects of lower values reported) (OR 1.77; 95% CI 1.29-0.2.42) and UtA-RI (per SD of MoM) (OR 1.28 95% CI 1.01-1.61) were found as significant variables in logistic regression. A prediction model with the variables family history thrombotic disease, HDL cholesterol and PIGF (OR 0.6; 95% CI 0.4-0.7) were selected by stepwise procedure. The final model had an area under the receiver operating characteristic curve (AUROC) of 0.68 (95% CI 0.62-0.75). Detection rates were 25.9% and 53.2% for a false positive rate of 10% and 25%, respectively.

It is an object of the invention to overcome at least one of the above-referenced problems.

### SUMMARY OF THE INVENTION

The Applicant realized that pre-eclampsia in the obese is most likely a different type of pre-eclampsia, whereby its prediction would require for factors that are specific to the disease within the obese pregnant population only. Furthermore, the Applicant has discovered that within the cohort of obese pregnant women, sub-cohorts of obese pregnant women exist for whom the pathology of development of pre-eclampsia is different, and for whom different metabolite biomarkers are relevant at an early stage of pregnancy. For example, the Applicant has discovered that the metabolite biomarkers; glycyl-glycine; taurine, and asymmetric dimethylarginine are important biomarkers for the early prediction of PE in obese pregnant women that are at risk of gestational diabetes, whereas for pregnant obese women that have a BMI>=40 (i.e. morbidly obese), the metabolite biomarkers bilirubin, biliverdin, etiocholanolone glucuronide, L-leucine, taurine, and L-arginine are important. The Applicant has therefore identified a panel of metabolite biomarkers for use in the early prediction of PE in an obese pregnant woman, from which specific combinations of biomarkers may be chosen and employed depending on a characteristic (patient parameter) of the woman such as ethnicity (black or non-black ethnicity), number of pregnancies (nulliparous v multiparous), risk of gestational diabetes, sex of foetus, and level of obesity (morbidly obese). Thus, the methods of the invention involve broadly involve:
assaying a biological sample obtained from the pregnant obese woman at an early stage of pregnancy to determine abundance values for a panel of obese pregnancy specific metabolite biomarkers;
determining a patient parameter for the woman (for example, BMI);
picking a subset of the panel of metabolite biomarkers based on the patient parameter; and
correlating abundance values for the subset of metabolites with risk of PE.

A computation model may be employed to identify a subset of metabolites based on the patient parameter, and then correlation of abundance values for the subset of metabolites with risk of PE. Combinations of metabolite biomarkers that are predictive of PE at an early stage of pregnancy are described herein, including combinations that are specified for different sub-types of obese pregnancies.

The Applicant has therefore developed a primarily metabolite-driven method of early detection of risk of PE that is tailored for obese pregnant women, and that can be further tailored for specific sub-cohorts of pregnant obese women, for example nulliparous or multi-parous pregnancies, morbidly obese pregnant women, women of black ethnicity, and pregnant women at risk of developing gestational diabetes. The methods employ a specific panel of obese pregnancy specific metabolites, that may be used singly, or preferably in groups, to stratify a pregnant obese woman according to risk of PE. With the current realisation that treatment for PE often needs to be tailored to the specific type of PE, the methods of the invention will help improve the stratification of obese pregnant women according to risk, and according to treatment regimen.

In a first aspect, the invention provides a computer implemented method of early prediction of risk of pre-eclampsia (including term PE or pre-term PE) in a pregnant obese woman, comprising the steps of:
inputting abundance values for a panel of obese pregnancy specific metabolite biomarkers obtained from an assayed biological sample into a computational model, in which the biological sample is obtained from an obese pregnant woman at 8 to 22 or 24 weeks of pregnancy; and
inputting a patient parameter for the pregnant obese woman selected from at least one of ethnicity, risk of gestational diabetes, fetal sex, number of pregnancies and level of obesity into the computational model,
   in which the computational model is configured to select a subset comprising at least one, and generally two, of the obese pregnancy specific metabolite biomarkers based on the patient parameter input, calculate a predicted risk of pre-eclampsia based on the abundance values for the subset of obese pregnancy specific metabolite biomarkers, and output the predicted risk of pre-eclampsia for the pregnant obese woman.

In one embodiment, the panel of obese pregnancy specific metabolite biomarkers comprises biliverdin; glycyl-glycine; NG-monomethyl-L-arginine; and dilinoleoyl glycerol.

In one embodiment, the panel of obese pregnancy specific metabolite biomarkers comprises biliverdin; glycyl-glycine; NG-monomethyl-L-arginine; dilinoleoyl glycerol; taurine, stearic acid. eicosapentaenoic acid, etiocholanolone glucuronide, L-(+)-ergothioneine,

In one embodiment, the panel of obese pregnancy specific metabolite biomarkers includes substantially all of: biliverdin; Met_XXX; glycyl-glycine; NG-monomethyl-L-arginine; etiocholanolone glucuronide; eicosapentaenoic acid; dilinoleoyl glycerol; L-leucine; hexadecanoic acid; 25-hydroxyvitamin D3; linoleic acid; octadecenoid acid; stearoylcarnitine; stearic acid; 8, 11, 14 eicosatrienoic acid; 1-palmitoyl-2-hydroxy-sn-glycero-3-phosphocholine; L-isoleucine; bilirubin; L-arginine; L-(+)-ergothioneine; myristic acid; L-palmitoylcarnitine; arachidonic acid, urea; choline; taurine; docosahexaenoic acid; asymmetric dimethylarginine; L-methionine; 2-hydroxybutanoic acid; 3-hydroxybutanoic acid; L-acetylcarnitine; citrulline; decanoylcarnitine; dodecanoyl-I-carnitine; and sphingosine-1-phosphate.

In one embodiment, the method includes a step of inputting the abundance value for obese pregnancy specific proteins into the computational model selected from PIGF, soluble endoglin and PAPPA, in which the computational model is configured to select a combination comprising a protein and a subset comprising at least one or two of the panel of obese pregnancy specific metabolite biomarkers based on the patient parameter input, correlate abundance values for the combination with risk of pre-eclampsia, and output a predicted risk of pre-eclampsia for the pregnant obese woman.

In one embodiment, the method includes a step of inputting a clinical risk factor value into the computational model, in which the computational model is configured to select a combination comprising a clinical risk factor value (for example blood pressure) and a subset comprising at least one or two of the panel of obese pregnancy specific metabolite biomarkers based on the patient parameter input, correlate abundance values for the combination with risk of pre-eclampsia, and output a predicted risk of pre-eclampsia for the pregnant obese woman.

In one embodiment, the patient parameter for the pregnant obese woman inputted into the computational model is risk of gestational diabetes, and in which the computation model is configured to select a subset of the panel of metabolite biomarkers comprising at least one of biliverdin; glycyl-glycine; taurine, and stearic acid. In one embodiment, a combination of a subset of metabolite biomarkers and optionally one of more clinical risk factor values selected of Table 18, or from the combinations below, is selected:
stearic acid, hexadecenoic acid and Mat_prev_pet;
biliverdin, citrulline, 25-hydroxyvitamin D3, and etiocholanolone glucuronide;
stearic acid, 8, 11, 14-eicosatrienoic acid and Mat_prev_pet; and
glycyl-glycine and asymmetric dimethylarginine.

In one embodiment, the patient parameter for the pregnant obese woman inputted into the computational model is no risk of gestational diabetes, and in which the computation model is configured to select a subset of the panel of metabolite biomarkers and optionally one of more clinical risk factor values from the combinations of Table 17.

In one embodiment, the patient parameter for the pregnant obese woman inputted into the computational model is that the pregnancy is nulliparous, and in which the computation model is configured to select a subset of the panel of metabolite biomarkers comprising at least one of biliverdin and etiocholanolone glucuronide. In one embodiment, the computation model is configured to select a subset of metabolite biomarkers and optionally one of more clinical risk factor values selected from Table 19, or the following combinations: eicosapentaenoic acid, biliverdin, etiocholanolone glucuronide and Mat_age_LT25; and biliverdin, etiocholanolone glucuronide, L-(+)-ergothioneine, and Mat_age_LT25.

In one embodiment, the patient parameter for the pregnant obese woman inputted into the computational model is that the pregnancy is multiparous, and in which the computation model is configured to select a subset of the panel of metabolite biomarkers of Table 20 or comprising at least one of biliverdin, glycyl-glycine, and L-(+)-ergothioneine. In one embodiment, the subset of metabolite biomarkers is selected from the following combinations:
L-arginine, glycyl-glycine, NG-monomethyl-L-arginine, and asymmetric dimethylarginine; and
8, 11, 14-eicosatrienoic acid, eicosapentaenoic acid, biliverdin, etiocholanolone glucuronide.

In one embodiment, the patient parameter for the pregnant obese woman inputted into the computational model is that the obese pregnant woman has a BMI> or =40, and in which the computation model is configured to select a subset of the panel of metabolite biomarkers comprising at least one of biliverdin, taurine, glycyl-glycine, and L-arginine. In one embodiment, the subset of metabolite biomarkers and optionally one of more clinical risk factor values selected from the combinations of Table 21 are inputted into the computational model.

In one embodiment, the patient parameter for the pregnant obese woman inputted into the computational model is that the obese pregnant woman is of black ethnicity, and in which the computation model is configured to select a subset of the panel of metabolite biomarkers of Table 22 or comprising at least one of taurine and stearic acid. In one embodiment, the the computational model is configured to select a subset of metabolite biomarkers and optionally one of more clinical risk factor values selected from Table 22 or the following combinations:
taurine, stearoylcarnitine, and Mat_hdl; and
3-hydroxybutanoic acid and stearic acid.

In one embodiment, the patient parameter for the pregnant obese woman inputted into the computational model is that the obese pregnant woman is not of black ethnicity, and in which the computation model is configured to select a subset of the panel of metabolite biomarkers comprising at least one of biliverdin, NG-monomethyl-L-arginine, and glycyl-glycine. In one embodiment, the computational model is configured to select a subset of metabolite biomarkers and optionally one of more clinical risk factor values selected from the combinations of Table 23.

In one embodiment, the patient parameter for the pregnant obese woman inputted into the computational model is that obese pregnant woman is carrying a male offspring, and in which the computation model is configured to select a subset of the panel of metabolite biomarkers comprising at least one of biliverdin and Met_XXX. In one embodiment, the computational model is configured to select the subset of metabolite biomarkers and optionally one of more clinical risk factor values selected from the combinations of Table 24.

In one embodiment, the patient parameter for the pregnant obese woman inputted into the computational model is that obese pregnant woman is carrying a female offspring, and in which the computation model is configured to select a subset of the panel of metabolite biomarkers comprising at least one of L-leucine, biliverdin, NG-monomethyl-L-arginine, and glycyl-glycine. In one embodiment, the computational model is configured to select the subset of metabolite biomarkers and optionally one of more clinical risk factor values selected from the combinations of Table 25.

In one embodiment, the computation model is configured to select a combination of plurality of metabolite biomarkers and optionally one of more clinical risk factor values selected from the combinations of Table 26 are inputted into the computational model.

In one embodiment, the computational model is configured to (a) combine the abundance values of the subset of metabolites and optionally the clinical risk factor values into a risk score using a multivariable algorithm, (b) compare the risk score with a reference risk score, and (c) output a predicted risk of pre-eclampsia based on the comparison.

In another aspect, the invention provides a method, generally a computer implemented method, of predicting risk of pre-eclampsia in a pregnant obese woman, comprising the steps of:
assaying a biological sample obtained from the pregnant obese woman (generally at an early stage of pregnancy, i.e. prior to the appearance of clinical symptoms, for example at 8 to 22 or 24 weeks of pregnancy) to determine an abundance of a plurality of metabolite biomarkers selected from the metabolites of Table 15, and preferably from the group consisting of:
   biliverdin; Met_XXX; glycyl-glycine; NG-monomethyl-L-arginine; etiocholanolone glucuronide; eicosapentaenoic acid; dilinoleoyl glycerol; L-leucine; hexadecanoic acid; 25-hydroxyvitamin D3; linoleic acid; octadecenoic acid; stearoylcarnitine; stearic acid; 8, 11, 14-eicosatrienoic acid; 1-palmitoyl-2-hydroxy-sn-glycero-3-phosphocholine; L-isoleucine; bilirubin; L-arginine; L-(+)-ergothioneine; myristic acid; L-palmitoylcarnitine; arachidonic acid, urea; choline; taurine; docosahexaenoic acid; asymmetric dimethylarginine; L-methionine; 2-hydroxybutanoic acid; 3-hydroxybutanoic acid; L-acetylcarnitine; citrulline; decanoylcarnitine; dodecanoyl-I-carnitine; sphingosine-1-phosphate, and
calculate a predicted risk of pre-eclampsia based on the abundance values for the plurality of metabolite biomarkers, that in one embodiment comprises inputting abundance values for the plurality of metabolite biomarkers into a computational model configured to calculate risk of risk of pre-eclampsia based the abundance values for the plurality of metabolite biomarkers.

In one embodiment, the plurality of metabolite biomarkers includes at least one, two, three or all of biliverdin; glycyl-glycine; NG-monomethyl-L-arginine; dilinoleoyl glycerol; taurine, and stearic acid.

In one embodiment, the plurality of metabolite biomarkers includes at least one, two, three or all of biliverdin; glycyl-glycine; NG-monomethyl-L-arginine; etiocholanolone glucuronide) and dilinoleoyl glycerol.

In one embodiment, the method comprises a step of determining an abundance of an obese pregnancy specific protein biomarker of PE, i.e. PIGF, s-Endoglin and/or PAPPA protein, and inputting the abundance value for the protein biomarker into the computational model, in which the computational model is configured to correlate the metabolite abundance values and the protein biomarker abundance value with risk of pre-eclampsia, and output a predicted risk of pre-eclampsia.

In one embodiment, the method comprises a step of inputting a clinical risk factor value into the computational model, in which the computational model is configured to correlate the metabolite abundance values and the clinical risk factor value with risk of pre-eclampsia, and output a predicted risk of pre-eclampsia.

In one embodiment, the obese pregnant woman is determined to have, or be at risk of developing, gestational diabetes, in which the plurality of metabolite biomarkers includes at least one of biliverdin; glycyl-glycine; taurine, and stearic acid.

In one embodiment, a combination of plurality of metabolite biomarkers and optionally one of more clinical risk factor values selected from the following combinations are inputted into the computational model:
stearic acid, hexadecenoic acid and Mat_prev_pet;
biliverdin, citrulline, 25-hydroxyvitamin D3, and etiocholanolone glucuronide;
stearic acid, 8, 11, 14-eicosatrienoic acid and Mat_prev_pet; and
glycyl-glycine and asymmetric dimethylarginine.

In one embodiment, the obese pregnant woman is nulliparous, in which the plurality of metabolite biomarkers includes at least one or both of biliverdin and etiocholanolone glucuronide.

In one embodiment, the obese pregnant woman is nulliparous, in which a combination of plurality of metabolite biomarkers and optionally one of more clinical risk factor values selected from the following combinations are inputted into the computational model: eicosapentaenoic acid, biliverdin, etiocholanolone glucuronide and Mat_age_LT25; and biliverdin, etiocholanolone glucuronide, L-(+)-ergothioneine, and Mat_age_LT25.

In one embodiment, the obese pregnant woman is multiparous, in which the plurality of metabolite biomarkers includes at least one of biliverdin, glycyl-glycine, and L-(+)-ergothioneine.

In one embodiment, the obese pregnant woman is multiparous, in which the plurality of metabolite biomarkers is selected from the following combinations:
L-arginine, glycyl-glycine, NG-monomethyl-L-arginine, and asymmetric dimethylarginine; and
8, 11, 14-eicosatrienoic acid, eicosapentaenoic acid, biliverdin, etiocholanolone glucuronide.

In one embodiment the obese pregnant woman is morbidly obese (i.e. has a BMI>=40), in which the plurality of metabolite biomarkers includes at least one or more of biliverdin, taurine, glycyl-glycine, and L-arginine.

In one embodiment the obese pregnant woman is morbidly obese, in which a combination of plurality of metabolite biomarkers and optionally one of more clinical risk factor values selected from the combinations of Table 21 are inputted into the computational model.

In one embodiment, the obese pregnant woman is of black ethnicity in which the plurality of metabolite biomarkers includes at least one or more of taurine and stearic acid.

In one embodiment, the obese pregnant woman is of black ethnicity, and in which a combination of plurality of metabolite biomarkers and optionally one of more clinical risk factor values selected from the following combinations are inputted into the computational model:
taurine, stearoylcarnitine, and Mat_hdl; and 3-hydroxybutanoic acid and stearic acid.

In one embodiment, the obese pregnant woman is not of black ethnicity, in which the plurality of metabolite biomarkers includes at least one or more of biliverdin, NG-monomethyl-L-arginine, and glycyl-glycine,

In one embodiment, the obese pregnant woman is not of black ethnicity, in which a combination of plurality of metabolite biomarkers and optionally one of more clinical risk factor values selected from the combinations of Table 23 are inputted into the computational model:

In one embodiment, the obese pregnant woman is carrying a male offspring, in which the plurality of metabolite biomarkers includes at least one or more of biliverdin and Met_XXX.

In one embodiment, the obese pregnant woman is carrying a male offspring, in which a combination of plurality of metabolite biomarkers and optionally one of more clinical risk factor values selected from the combinations of Table 24 are inputted into the computational model.

In one embodiment, the obese pregnant woman is carrying a female offspring, in which the plurality of metabolite biomarkers includes at least one or more of L-leucine, biliverdin, NG-monomethyl-L-arginine, and glycyl-glycine,

In one embodiment, the obese pregnant woman is carrying a female offspring, in which a combination of plurality of metabolite biomarkers and optionally one of more clinical risk factor values selected from the combinations of Table 25 are inputted into the computational model.

In one embodiment, a combination of plurality of metabolite biomarkers and optionally one of more clinical risk factor values selected from the combinations of Table 26 are inputted into the computational model.

In one embodiment, the computational model is configured to (a) combine the abundance values of the plurality of metabolites (and optionally one or more protein biomarkers and/or clinical risk factors) into a risk score using a multivariable algorithm, (b) compare the risk score with a reference risk score, and (c) output a predicted risk of pre-eclampsia based on the comparison.

In any embodiment, the predicted risk of pre-eclampsia is prediction of risk of preterm pre-eclampsia.

In any embodiment, the predicted risk of pre-eclampsia is prediction of risk of term pre-eclampsia.

In any embodiment, the predicted risk of pre-eclampsia is prediction of high risk of pre-eclampsia.

In any embodiment, the predicted risk of pre-eclampsia is prediction of low risk of pre-eclampsia.

In another aspect, the invention provides a computer implemented method of early prediction of risk of pre-eclampsia in a plurality of pregnant obese women including a first obese pregnant woman having a first patient parameter and a second obese pregnant woman having a second patient parameter that is different to the first patient parameter, comprising the steps of:
predicting risk of pre-eclampsia in the first patient according to a method of the invention; and
predicting risk of pre-eclampsia in the second patient according to a method of the invention.

In another aspect, the invention provides a computer implemented method of early prediction of risk of pre-eclampsia in a plurality of pregnant obese women including a first obese pregnant woman having a first patient parameter, a second obese pregnant woman having a second patient parameter that is different to the first patient parameter, and a third obese pregnant woman having a third patient parameter that is different to the first and second patient parameters, the method comprising the steps of:
predicting risk of pre-eclampsia in the first patient according to a method of the invention;
predicting risk of pre-eclampsia in the second patient according to a method of the invention; and
predicting risk of pre-eclampsia in the third patient according to a method of the invention.

In another aspect, the invention provides a method of identifying at an early stage of pregnancy elevated risk of a pregnant obese woman developing pre-eclampsia, comprising the steps of:
providing the sex of the foetus; and
providing the BP of the pregnant obese woman;
   wherein when the fetal sex is male and the blood pressure is elevated relative to a reference blood pressure value for a pregnant obese woman, the pregnant obese woman exhibits an increased risk of developing pre-eclampsia, an/or
   wherein when the fetal sex is male and the blood pressure is reduced relative to a reference blood pressure value for a pregnant obese woman, the pregnant obese woman exhibits a decreased risk of developing pre-eclampsia.

Thus, for women with male offspring, blood pressure can be used as a prognostic variable of risk of pre-eclampsia. The reference blood pressure value is generally the average blood pressure value for pregnant obese women who do not develop pre-eclampsia, where a blood pressure value higher than the average indicates elevated risk of PE compared with the general sub-population of pregnant obese women, and a blood pressure value lower than the average indicates reduced risk of PE compared with the general sub-population of pregnant obese women.

In one embodiment, the method comprises a step of determining the sex of the foetus.

In one embodiment, the sex of the foetus is determined by means of in-vitro diagnosis of a sample obtained from the pregnant woman at an early stage of pregnancy.

In one embodiment, the method comprises the steps of:
assaying a biological sample obtained from the pregnant obese woman at an early stage of pregnancy to determine an abundance of one or more obese pregnancy specific metabolite biomarkers, and
quantifying the risk of pre-eclampsia based on the abundance values for the one or more obese pregnancy specific metabolite biomarkers.

In one embodiment, the obese pregnancy specific metabolite biomarkers are selected from the group consisting of:
biliverdin; Met_XXX; glycyl-glycine; NG-monomethyl-L-arginine; etiocholanolone glucuronide; eicosapentaenoic acid; dilinoleoyl glycerol; L-leucine; hexadecanoic acid; 25-hydroxyvitamin D3; linoleic acid; octadecenoic acid; stearoylcarnitine; stearic acid; 8, 11, 14-eicosatrienoic acid; 1-palmitoyl-2-hydroxy-sn-glycero-3-phosphocholine; L-isoleucine; bilirubin; L-arginine; L-(+)-ergothioneine; myristic acid; L-palmitoylcarnitine; arachidonic acid, urea; choline; taurine; docosahexaenoic acid; asymmetric dimethylarginine; L-methionine; 2-hydroxybutanoic acid; 3-hydroxybutanoic acid; L-acetylcarnitine; citrulline; decanoylcarnitine; 24 dodecanoyl-I-carnitine; sphingosine-1-phosphate.

In one embodiment, the obese pregnancy specific metabolite biomarkers selected from the combinations of Table 24.

In one embodiment, the method employs a computer processor configured to receive an input of the patient's blood pressure, fetal sex, and optionally an abundance of at least one obese pregnancy specific metabolite biomarker, correlate the fetal sex, blood pressure, and optionally the abundance of the at least one obese pregnancy specific metabolite biomarker, with risk of pre-eclampsia, and provide an output of risk of pre-eclampsia, the method comprising the steps:
inputting fetal sex into the computer processor;
inputting the patient's blood pressure into the computer processor;
optionally inputting an abundance of at least one obese pregnancy specific metabolite biomarkers into the computer processor,
correlating, by the computer processor, the inputs with risk of pre-eclampsia; and outputting, by the computer processor, risk of the patient developing pre-eclampsia.

In another aspect, the invention provides a computer program comprising program instructions for causing a computer to perform the method steps of a method of the invention.

In another aspect, the invention provides a computer implemented method of identifying at an early stage of pregnancy elevated risk of a pregnant obese woman developing pre-eclampsia, comprising the steps of:
providing the sex of a foetus; and
providing the BP of the pregnant obese woman; and
comparing the blood pressure value with a reference value for the blood pressure,
and output a predicted risk for pre-eclampsia based on the comparison.

In another aspect, the invention provides a computer program comprising program instructions for causing a computer to perform the method of the invention.

In another aspect, the invention provides a system configured to identify at an early stage of pregnancy elevated risk of a pregnant obese woman developing pre-eclampsia, the system comprising:
a device to determine the sex of a foetus; and
a device to measure the blood pressure of the pregnant obese woman; and
a processor module configured to receive the fetal sex and blood pressure inputs,
compare the blood pressure input with a reference blood pressure value, and
output the risk of pre-eclampsia based on the comparison.

When the blood pressure is elevated relative to the reference value, the output is generally an elevated risk of pre-eclampsia, or when the blood pressure is reduced relative to the reference value, the output is generally a reduced risk of pre-eclampsia.

In another aspect, the invention provides a method of stratifying an obese pregnant woman according to a prophylactic treatment regimen at an early stage of pregnancy (i.e. prior to the appearance of symptoms of pre-eclampsia), comprising the steps of predicting risk of pre-eclampsia in the pregnant woman according to a method of the invention, and stratifying the obese pregnant woman into a prophylactic treatment regimen according to detected risk of PE.

In another aspect, the invention provides a method (for example a computer implemented method) of predicting risk of pre-eclampsia in a pregnant obese woman, comprising a step of assaying a biological sample obtained from the pregnant obese woman at an early stage of pregnancy (i.e. prior to the appearance of symptoms of pre-eclampsia, for example at 8 to 22 weeks of pregnancy) to determine an abundance of at least one metabolite biomarker selected from Table 15 or from the group consisting of:
NG-Monomethyl-L-arginine, Biliverdin, Glycyl-glycine, Dilinoleoyl-glycerol, Etiocholanolone glucuronide, Bilirubin, L-leucine, 25-Hydroxyvitamin D3, 1-heptadecanoyl-2-hydroxy-sn-glycero-3-phosphocholine, L-(+)-Ergothioneine, octadecenoic acid, Met_XXX, Hexadecanoic acid, Linoleic acid, L-Isoleucine, Stearic acid, Asymmetric dimethylarginine, Taurine, 8,11,14 Eicosatrienoic acid, L-alanine, L-arginine, Stearoylcarnitine and Urea.
   wherein a modulated abundance of the at least one metabolite biomarker relative to a reference abundance for that biomarker correlates with a risk of the pregnant obese woman developing pre-eclampsia.

In one embodiment, the method comprises inputting an abundance value for the metabolite biomarkers into a computational model configured to compare the abundance values with a reference value for the metabolite biomarker, and output a predicted risk of pre-eclampsia based on the comparison.

In one embodiment, the at least one metabolite biomarker is selected from the group consisting of: NG-Monomethyl-L-arginine, Glycyl-glycine, Dilinoleoyl-glycerol, Biliverdin, Asymmetric dimethylarginine, Taurine, Bilirubin, Etiocholanolone glucuronide, L-leucine, L-(+)-Ergothioneine, and 1-heptadecanoyl-2-hydroxy-sn-glycero-3-phosphocholine.

In one embodiment, the at least one metabolite biomarker is selected from the group consisting of: NG-Monomethyl-L-arginine, Biliverdin, Bilirubin, and L-(+)-Ergothioneine.

In one embodiment, the obese pregnant woman is determined to not have, and not be at risk of developing, gestational diabetes, in which the at least one metabolite biomarker is selected from NG-Monomethyl-L-arginine, Dilinoleoyl-glycerol, Biliverdin, Etiocholanolone glucuronide, Bilirubin, L-leucine, Glycyl-glycine, linoleic acid, Hexadecanoic acid, Met_XXX, Octadecenoic acid, Stearic acid.

In one embodiment, the at least one metabolite biomarker is selected from the group consisting of: NG-Monomethyl-L-arginine, Dilinoleoyl-glycerol, Octadecenoic acid, Hexadecanoic acid, Linoleic acid and Stearic acid.

In one embodiment, the obese pregnant woman is determined to have, or be at risk of developing, gestational diabetes, in which the at least one metabolite biomarker is selected from Asymmetric dimethylarginine, taurine and NG-Monomethyl-L-arginine.

In one embodiment, the at least one metabolite biomarker is selected from the group consisting of: Asymmetric dimethylarginine and taurine.

In one embodiment, the obese pregnant woman is nulliparous, in which the metabolite biomarker is selected from the group consisting of NG-Monomethyl-L-arginine, Biliverdin, Dilinoleoyl-glycerol, L-leucine, L-alanine, L-Isoleucine, Etiocholanolone glucuronide, Bilirubin.

In one embodiment, the at least one metabolite biomarker is selected from the group consisting of NG-Monomethyl-L-arginine, Biliverdin.

In one embodiment, the obese pregnant woman is multiparous, in which the metabolite biomarker is selected from the group consisting of Glycyl-glycine, 25-Hydroxyvitamin D3, Bilirubin, Etiocholanolone glucuronide, 8,11,14 Eicosatrienoic acid, Dilinoleoyl-glycerol, L-(+)-Ergothioneine.

In one embodiment, the metabolite biomarker is selected from 8,11,14 Eicosatrienoic acid, and Glycyl-glycine.

In one embodiment, the obese pregnant woman has a BMI>=40, in which the metabolite biomarker is selected from the group consisting of Bilirubin, Biliverdin, L-arginine, Etiocholanolone glucuronide, L-leucine, taurine.

In one embodiment, the metabolite biomarker is selected from Bilirubin, Biliverdin, taurine and L-arginine.

In one embodiment, the obese pregnant woman is of black ethnicity, in which the metabolite biomarker is selected from L-(+)-Ergothioneine and stearylcarnitine.

In one embodiment, the obese pregnant woman is of non-black ethnicity, in which the metabolite biomarker is selected from NG-Monomethyl-L-arginine, Glycyl-glycine, Biliverdin, Etiocholanolone glucuronide, Dilinoleoyl-glycerol, L-leucine, Met_XXX, 25-Hydroxyvitamin D3, Bilirubin, L-Isoleucine, Octadecenoic acid, 1-heptadecanoyl-2-hydroxy-sn-glycero-3-phosphocholine, Hexadecanoic acid, linoleic acid

In one embodiment, the metabolite biomarker is selected from Glycyl-glycine linoleic acid, Met_XXX, NG-Monomethyl-L-arginine, L-Isoleucine

In one embodiment, the method comprises a step of determining an abundance of a protein biomarker of PE, i.e. PIGF, PAPPA protein and/or soluble endoglin, and inputting the abundance value for the protein biomarker into the computational model, in which the computational model is configured to correlate the metabolite abundance value and the protein biomarker abundance value with risk of pre-eclampsia, and output a predicted risk of pre-eclampsia.

In one embodiment, the method comprises a step of inputting a clinical risk factor value into the computational model, in which the computational model is configured to correlate the metabolite abundance values and the clinical risk factor value with risk of pre-eclampsia, and output a predicted risk of pre-eclampsia. The clinical risk factor may be selected from any of Table 7.

There is also provided a computer program comprising program instructions for causing a computer program to carry out the above methods which may be embodied on a record medium, carrier signal or read-only memory.

In one embodiment, the methods include an assaying step, which in one embodiment comprises assaying the biological sample with mass spectrometry.

In one embodiment, the assaying step comprises treating the sample by liquid chromatography (LC) to provide a mass spectrometry compatible eluent containing resolved metabolites, and assaying the eluent using tandem mass spectrometry.

In one embodiment, the assaying step comprises an initial step of pre-treating the biological sample by precipitation with a metabolite extraction solvent or/and by solid phase extraction to provide a pre-treated sample.

In one embodiment, the method employs LC directly hyphenated to MS, hereafter “in-line LC-MS” or “LC-MS”.

In one embodiment, the metabolite extraction solvent comprised methanol, isopropanol and a buffer, typically a volatile buffer, ideally an ammonium acetate buffer.

In one embodiment, the method comprises the steps of: providing an absorptive sampling device comprising the biological sample absorbed on an absorptive medium; extracting the biological sample from the absorptive sampling device into a metabolite extraction solvent to provide a mixture, separating a metabolite rich supernatant from the mixture, and performing a liquid chromatography (LC) step on the supernatant to provide a mass spectrometry compatible eluent containing multiple metabolites.

In one embodiment, the absorptive sampling device is a volume controlling sampling device, for example a VAMS device. Examples of absorptive sampling devices and volume controlling absorptive sampling devices are provided herein. In this aspect of the invention, a fixed volume of biological sample is collected and processed into a metabolite rich eluent.

In one embodiment, the metabolite extraction solvent comprises methanol, isopropanol and an ammonium acetate buffer.

In one embodiment, the metabolite extraction solvent comprises methanol, isopropanol and an ammonium acetate buffer in a ratio of about 10:9:1 (v/v/v).

In one embodiment, the biological sample is extracted from the absorptive sampling device directly into the metabolite extraction solvent.

Typically, the LC step is a dual LC step in which a first aliquot of the sample is subjected to a separation process using one form of LC to provide a first mass spectrometry compatible eluent in which metabolites of a first type are resolved from each other (e.g. hydrophobic metabolites), and a second aliquot of the sample is subjected to a separation process using a second form of LC to provide a second mass spectrometry compatible eluent in which metabolites of a second type are resolved from each other (e.g. hydrophilic metabolites). Mixtures of hydrophobic metabolites within a sample can be resolved by employing reverse phase LC (for instance, C18-, C8-, C4-, cyano-, phenyl-, biphenyl-, pentafluorophenyl-bonded phases). Mixtures of hydrophilic metabolites within a sample can be resolved by employing hydrophilic interaction LC (for instance, bare silica, diol-bonded-phase, amide-polyol-bonded, zwitter-ionic etc).

Thus, in one embodiment, the LC step comprises separating a first aliquot of the pre-treated sample by reversed phase liquid chromatography to provide a first mass spectrometry compatible eluent containing resolved hydrophobic metabolites, separating a second aliquot of the pre-treated sample by hydrophilic interaction chromatography to provide a second mass spectrometry compatible eluent containing resolved hydrophilic metabolites, and assaying the first and second eluents using on-line tandem mass spectroscopy.

In one embodiment, the RPLC employs a varying mixture of a first mobile phase (A) comprising water, methanol and a volatile buffer (e.g. an ammonium acetate buffer) and a second mobile phase (B) comprising methanol, acetonitrile, isopropanol and a volatile buffer (i.e. an ammonium acetate buffer).

In one embodiment, the RPLC mobile phases are mixed according to a varying volumetric gradient of about 1-20% (preferably about 10%) to 80-100% (preferably about 100%) mobile phase B over a suitable period, for example 1-20 minutes or about 8-12 minutes, preferably about 10 minutes. The varying volumetric gradient may be a linear gradient, or a stepwise gradient.

In one embodiment, the HILIC employs a varying mixture of a first mobile phase (A) comprising a volatile ammonium formate buffer and a second mobile phase (B) comprising acetonitrile.

In one embodiment, the HILIC mobile phases are mixed according to a varying volumetric gradient of about 80-100% (preferably about 100%) to 40-60% (preferably about 50%) mobile phase B over a period of about 8-12 minutes, preferably about 10 minutes. The varying volumetric gradient may be a linear gradient, or a stepwise gradient.

In one embodiment the combined LC run would employ two columns of different polarities installed in parallel employing a multi-port valve, e.g., a 10-port valve, and two LC pump systems. During one single LC run the first injection would be made onto the hydrophilic column to carry out the separation of the hydrophilic metabolites. The eluent of this hydrophilic interaction column is connected to the mass spectrometer and the eluent of the reversed phase column is directed to waste. After the separation of the hydrophilic metabolites is finished, the multi-port valve is used to switch the eluent of the hydrophilic column to waste and the eluent of the reversed phase column to the mass spectrometer. Subsequently the same sample is injected again, now onto the hydrophobic column where the separation of the hydrophobic metabolites takes place.

In another setup the reversed phase column would be the first column and the hydrophilic interaction column would be the second column.

A list of mass spectrometry compatible buffers for employment in LC-MS can be found at https://www.nestgrp.com/protocols/trng/buffer.shtml

In one embodiment, the biological sample comprises at least one stable isotope-labelled internal standard (SIL-IS) corresponding to a hypertensive disorder of pregnancy relevant metabolite. In one embodiment, the at least one SIL-IS is added to the biological sample prior to pre-treatment with the metabolite extraction solvent. In one embodiment, a plurality of SIL-IS's are added to the sample. In one embodiment, the at least one SIL-IS corresponds to a hypertensive disorder of obese pregnancy relevant metabolite disclosed herein.

In one embodiment, the extraction solvent comprises methanol, isopropanol and buffer (for example a volatile buffer). In one embodiment, the buffer is an acetate buffer. In one embodiment, the acetate buffer is an ammonium acetate buffer. In one embodiment, the acetate buffer has a concentration of about 150-250 mM, preferably about 200 mM. In one embodiment, the buffer is configured to buffer the pH of the extraction solvent to about 4-5, preferably about 4.5. In one embodiment, the extraction solvent comprises methanol and isopropanol in a ratio of 5-15:5-15. In one embodiment, the extraction solvent comprises methanol and isopropanol in approximately equal amounts (i.e. 8-12:8-12). In one embodiment, the extraction solvent comprises methanol, isopropanol and acetate buffer in a ratio of about 10:9:1 (v/v/V).

In one embodiment, the metabolite extraction solvent comprises about 0.01% to 0.1% antioxidant (m/v). In one embodiment, the metabolite extraction solvent comprises about 0.05% antioxidant (vm/v). In one embodiment, the antioxidant is 3,5-Di-tert-4-butyl-hydroxytoluene BHT (CAS: 128-37-0). Other antioxidants that could be employed include e.g., a mix of Ascorbic acid (CAS: 50-81-7) with Ethylenediaminetetraacetic acid (EDTA; CAS: 60-00-4); butylated hydroxy anisole (BHA; CAS: 25013-16-5), Butylated hydroxy toluene which we use (BHT,CAS: 128-37-0), and propyl gallate (PG; CAS: 121-79-9).

In one embodiment, the metabolite extraction solvent is added to the biological sample in two separate aliquots and mixed after addition of the first aliquot and again after the addition of the second aliquot. In one embodiment, the solvent and sample are mixed after addition of the second aliquot. In one embodiment, the solvent and sample are mixed by vortexing.

In one embodiment, the mixture of biological sample and extraction solvent is incubated at a temperature of less than room temperature, for example less than 10° C. or 5° C. (i.e. typically less than −20° C., −10° C., −5° C., or 0° C.) for a period of time to assist protein precipitation, prior to separation of precipitated protein. In one embodiment, precipitated protein is separated by centrifugation to provide the pre-treated sample that is typically substantially free of protein and enriched in metabolites.

In one embodiment, the biological sample is a liquid sample and is collected and stored on volumetric absorptive microsampling (VAM) device. The Applicant has discovered that use of a VAM device provide for accurate control of blood sample volume, an important consideration for applications where accurate quantitative analysis of metabolites is required, such as detection or prediction of disease.

Thus, in one embodiment, the method includes the steps of providing a biological sample on an absorption medium as preferably collected with a volume-controlling sampling device which—by design—collects a controlled volume of the sample on a suitable absorption medium (for example, a volumetric absorptive microsampling device); and extracting the volumetrically obtained biological sample from such absorption medium device. In one embodiment, the biological sample is extracted from the absorption medium directly into the metabolite extraction solvent.

In one embodiment, the tandem mass spectroscopy is targeted tandem mass spectrometry. In one embodiment, the tandem mass spectrometry is carried out in multiple reaction monitoring mode.

In one embodiment, the tandem mass spectrometry comprises an ionisation technique enabling the direct analysis of an LC effluent, like electrospray ionization, and ionisation techniques derived there-of, atmospheric pressure chemical ionisation or atmospheric pressure photoionization, or continuous flow-fast atom bombardment.

When the methods of the invention are used in such way that the LC-eluent is fractionated, deposited in discrete droplets on a surface, or traced on a surface, to preserve the spatial resolution as achieved by the chromatography for later analysis, the tandem mass spectrometry can be performed using other ionization techniques also. Among them, for instance, electron ionization, chemical ionization, field desorption ionisation, matrix-assisted laser desorption ionization, surface enhanced laser desorption ionization.

In one embodiment, the tandem mass spectroscopy is carried out under both positive and negative electrospray ionization. The Applicant has discovered that in applications where multiple metabolites are being assayed, it is difficult to sufficiently charge all metabolites of interest when they all (or their fragments) need to carry the same charge. The Applicant has addressed this issue by employing a method in which the samples are assayed by tandem MS using both positive and negative electrospray ionization.

In one embodiment, the method is a method of profiling metabolites in the biological sample. In one embodiment, the method is a method of qualitative and/or quantitative profiling of metabolites in the biological sample. In one embodiment, the method is a method of qualitative and/or quantitative profiling of disorders of pregnancy related metabolites in the biological sample.

In one embodiment, the method is a method of qualitative and/or quantitative profiling of pre-eclampsia related metabolites in the biological sample.

In one embodiment, the method is a method of profiling metabolites selected from Table 3 (ANNEX), for example all or substantially all of the metabolites of Table 15 (ANNEX). In one embodiment, the method is a method of profiling all or substantially all of the metabolites:
25-Hydroxyvitamin D3; 2-Hydroxybutanoic acid; 3-Hydroxybutanoic acid; L-alanine; Arachidonic acid; L-arginine; L-leucine; 8,11,14 Eicosatrienoic acid; Citrulline; Decanoylcarnitine; Dodecanoyl-I-carnitine; Docosahexaenoic acid; Dilinoleoyl-glycerol (the total of the metabolites: 1,3Dilinoleoyl-glycerol; 1,2-Dilinoleoyl-glycerol (isomer mixture)); Choline; Glycyl-glycine; Homo-L-arginine; Hexadecanoic acid (palmitic acid); L-Isoleucine; Linoleic acid; L-methionine; NG-Monomethyl-L-arginine; Octadecenoic acid (the total of the metabolites: Oleic acid (primary contributor; (Z)-octadec-9-enoic acid), and possibly (E)-octadec-9-enoic acid, (Z,E)-octadec-11-enoic acid, (Z,E)-octadec-6-enoic acid); L-Palmitoylcarnitine; Asymmetric dimethylarginine; Sphingosine 1-phosphate; Sphinganine-1-phosphate (C18 base); Symmetric dimethylarginine; Taurine; Urea; Stearoylcarnitine; Eicosapentaenoic acid; 1-heptadecanoyl-2-hydroxy-sn-glycero-3-phosphocholine; Bilirubin; Biliverdin; Etiocholanolone glucuronide; Cotinine; Myristic acid; Stearic acid; L-(+)-Ergothioneine; Met_XXX (unknown metabolite signal corresponding a molecular mass of 366); 1-Palmitoyl-2-hydroxy-sn-glycero-3-phosphocholine (LysoPC(16:0)); L-Acetylcarnitine; L-Lysine; L-Glutamine

In one embodiment, the biological sample is a liquid, for example blood, or a blood derivative such as serum or plasma, as well as urine, sweat, saliva, tears, amniotic fluid, cerebrospinal fluid, or nipple aspirate. In one embodiment, the biological sample is obtained from a pregnant woman.

In one embodiment, the methods comprise methods for early prediction of risk of PE in a pregnant obese woman, wherein the sample is preferably obtained from the pregnant obese woman at 8 to 24 weeks' gestation. In one embodiment, the sample is obtained from the pregnant obese woman in the second trimester of pregnancy, although the sample can be obtained in the first or third trimester. In one embodiment, the sample is obtained from the pregnant obese woman at 16+/−2 or 3 weeks' gestation (i.e. 13-19 weeks or 14-18 weeks' gestation).

In one embodiment, the method of the invention is a method of identifying a pregnant obese woman, ideally in the second trimester of pregnancy, suitable for treatment with a prophylactic therapy/intervention for preventing development of PE, for example, Metformin treatment.

In one embodiment, a metabolite biomarker, or combination of metabolite biomarkers, is chosen such that detection of modulated abundance of the biomarker or biomarkers correlates with an AUROC score of at least 0.60, 0.61, 0.62, 0.63, 0.64, 0.65, 0.66, 0.67, 0.68, 0.69, 0.70, 0.75, 0.80, 0.85, or 0.90

The invention may be primarily employed as a means of assessment of the risk, or aiding in the assessment of the risk, that a pregnant obese woman will develop pre-eclampsia. However, the invention may also be employed as a means of diagnosing the presence of pre-eclampsia, predicting the risk of pre-eclampsia, assessment of likely patient outcome due to the syndrome, and assessment of effectiveness of a treatment for pre-eclampsia in which the levels of one or more of the metabolite markers of the invention assessed over a period of treatment to monitor for effectiveness of the treatment, all in an obese sub-population of pregnant women. In terms of prediction, the methods of the invention typically provide for a detection rate of at least 40%, 50%, 60% or 70% in which the false positive rate is at most 20% when all metabolite classes are employed. Further, the methods of the invention typically provide for a detection rate of at least 60%, 70%, 80% or 90% in which the false positive rate is at most 40% when all metabolite classes are employed.

In one embodiment of the invention, the method is a method of monitoring the effectiveness of a treatment for a pre-eclampsia in a pregnant obese woman, in which changes in the abundance of a metabolite or a plurality of metabolites according to the invention is correlated with effectiveness of the treatment. For example, when the metabolite being assayed is Etiocholanolone glucuronide, then a decrease in abundance of the metabolite is generally indicative of effectiveness of the treatment. Generally, the method will involve an initial assay to determine the starting abundance level of the or each metabolite, and then further periodic measurements of the abundance level of the or each metabolite during and/or after the course of the treatment to monitor abundance levels of the or each marker. Likewise, when the metabolite being assayed is NG-Monomethyl-L-arginine, then a decrease in abundance of the metabolite is generally indicative of effectiveness of the treatment. Generally, the method will involve an initial assay to determine the starting abundance level of the or each metabolite, and then further periodic measurements of the abundance level of the or each metabolite during and/or after the course of the treatment to monitor abundance levels of the or each marker.

Preferably, the step of determining the quantitative estimate of pre-eclampsia risk in a pregnant obese woman comprises determining the likelihood of pre-eclampsia using a multivariable analysis which typically comprises using the abundance of the or each metabolite biomarker and distribution parameters derived from a set of reference abundance values. Ideally, the multivariable analysis employs logistic regression. Other methods such as partial least square-discriminant analysis (PLS-DA), Principal Component Canonical Variants Analysis (PC-CVA), Bayesian inference, boosted regression or others could be used for the purpose.

The methods of the invention relate to the early prediction of pre-eclampsia in pregnant obese women.

However, the methods of the invention are also applicable for the early prediction of risk of hypertensive disorders in pregnant obese women, including for example eclampsia, mild pre-eclampsia, severe pre-eclampsia, early onset pre-eclampsia, i.e., pre-eclampsia presenting and/or requiring for pre-eclampsia induced delivery before 32 or 34 weeks of gestation, preterm pre-eclampsia, i.e., pre-eclampsia presenting and/or requiring for pre-eclampsia induced delivery before 37 weeks of gestation, term pre-eclampsia, i.e., pre-eclampsia presenting and/or requiring for pre-eclampsia induced delivery at or after 37 weeks of gestation, post-partum pre-eclampsia presenting after delivery, chronic hypertension, EPH gestosis, gestational hypertension, superimposed pre-eclampsia, HELLP syndrome, or nephropathy. Further, while the invention is described with reference to pregnant obese humans, it is also applicable to pregnant obese higher mammals.

In another aspect, the invention relates to a method of treating a pregnant obese woman identified as being at risk of developing pre-eclampsia to prevent development of pre-eclampsia, the method comprising identifying a pregnant obese woman at risk of development of pre-eclampsia using a method of the invention, and then treating the pregnant obese woman with a prophylactic therapy.

In one embodiment, the prophylactic therapy is Metformin treatment. In one embodiment, the prophylactic therapy is administered to the pregnant obese woman during the first, second and/or third trimester of pregnancy.

In one embodiment, the pregnant obese woman is identified as being at risk of pre-eclampsia in the second trimester.

In one embodiment, the pregnant obese woman has a singleton pregnancy.

Other aspects and preferred embodiments of the invention are defined and described in the other claims set out below.

### BRIEF DESCRIPTION OF THE FIGURES

FIG. 1: Exemplary ROC curves for single predictors of PE in obese pregnant women across all viewpoints assessed.

FIG. 2: Exemplary ROC curves and algorithms for multivariable predictors of PE in obese pregnant women across all viewpoints assessed

FIG. 3: Exemplary ROC curves and algorithms for multivariable rule-in/Rule-out predictors of PE in obese pregnant women across all viewpoints assessed

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

Unless otherwise required by context, the use of the terms “AUROC” and “AUC” are used interchangeably herein.

As used herein, the term “risk of pre-eclampsia” should be understood to mean an increased risk of developing pre-eclampsia compared with the general sub-population of pregnant obese women. In one embodiment, the term implies an AUROC predictive performance of at least 0.65. In one embodiment, the term implies an AUROC predictive performance of at least 0.70. In one embodiment, the term implies an AUROC predictive performance of at least 0.75. In one embodiment, the term implies an AUROC predictive performance of at least 0.80. In one embodiment, the term implies an AUROC predictive performance of at least 0.85. In one embodiment, the term implies an AUROC predictive performance of at least 0.90.

As used herein, the term “early detection of risk of pre-eclampsia” means detection of risk prior to the appearance of symptoms of the syndrome, for example during the second trimester of pregnancy (or late first trimester or early third trimester), for example from 8 to 24 weeks, 8 to 22 weeks, or from 10 and 22 weeks, and ideally about 16 weeks (+/−2 or 3 weeks) gestation. Thus, metabolite abundance is determined in a sample obtained from the pregnant obese woman at an early stage of the pregnancy. The term “early stage of pregnancy” means prior to the appearance of clinical symptoms of the syndrome, for example during the second trimester of pregnancy, for example from 8 to 24 weeks or from 10 to 22 weeks, and ideally about 16 weeks (+/−2 or 3 weeks).

As used herein, the term “pre-eclampsia” means the disorder of pregnancy characterized by gestational hypertension accompanied by one or more of the following new-onset conditions at or after 20 weeks' gestation:
1. Proteinuria
2. Other maternal organ dysfunction, including:
   Acute kidney injury (AKI) (creatinine ≥90 μmol/L; 1 mg/dL)
   liver involvement (elevated transaminases e.g. ALT or AST>40 IU/L) with or without right upper quadrant or epigastric abdominal pain)
   neurological complications (examples include eclampsia, al-tered mental status, blindness, stroke, clonus, severe headaches, persistent visual scotomata)
   haematological complications (thrombocytopenia-platelet count below 150,000/L, DIC, hemolysis)
3. Uteroplacental dysfunction (such as fetal growth restriction, abnormal umbilical artery Doppler wave form analysis, or stillbirth), as defined by the International Society for the Study of Hypertension in Pregnancy³⁴. Other and/or older pre-eclampsia diagnostic criteria are also comprised within this application, for instance, but not limited to the previous International Society for the Study of Hypertension in Pregnancy definition³⁵ or as summarised in Table 1 in Steegers et al.¹

The term “pre-eclampsia” includes both term pre-eclampsia and pre-term pre-eclampsia.

As used herein, the term “obese” as applied to a pregnant woman means the woman is obese as per the WHO definition. In one embodiment, obesity is determined just-prior to or during her pregnancy, or at 16 weeks of pregnancy (+/−2 or 3 weeks). The WHO defines obesity as follows: “obesity is the body mass index (BMI), a person's weight (in kilograms) divided by the square of his or her height (in metres). A person with a BMI of 30 or more is generally considered obese”. Where appropriate ethnicity specific bmi thresholds can also be considered, e.g, for Asian populations³⁶. The term “morbidly obese” refers to a pregnant obese woman who exhibits a BMI of >=40 at time of taking the biological sample.

As used herein, the term “biological sample” (or the test sample or the control) may be any biological fluid obtained from the subject pregnant woman or the foetus, including blood, serum, plasma, saliva, amniotic fluid, cerebrospinal fluid. Ideally, the biological sample is serum. The subject may be fasting or non-fasting when the biological sample is obtained. In a preferred embodiment, the biological sample is, or is derived from, blood obtained from the test pregnant obese woman.

In the specification, the term “panel of obese pregnancy specific metabolite biomarkers” refers to one or more of the metabolite biomarkers of Table 15 for example metabolite biomarker selected from: 25-Hydroxyvitamin D3; 2-Hydroxybutanoic acid; 3-Hydroxybutanoic acid; L-alanine; Arachidonic acid; L-arginine; L-leucine; 8,11,14 Eicosatrienoic acid; Citrulline; Decanoylcarnitine; Dodecanoyl-I-carnitine; Docosahexaenoic acid; Dilinoleoyl-glycerol (the total of the metabolites: 1,3Dilinoleoyl-glycerol; 1,2-Dilinoleoyl-glycerol (isomer mixture)); Choline; Glycyl-glycine; Homo-L-arginine; Hexadecanoic acid (palmitic acid); L-Isoleucine; Linoleic acid; L-methionine; NG-Monomethyl-L-arginine; Octadecenoic acid (the total of the metabolites: Oleic acid (primary contributor; (Z)-octadec-9-enoic acid), and possibly (E)-octadec-9-enoic acid, (Z,E)-octadec-11-enoic acid, (Z,E)-octadec-6-enoic acid); L-Palmitoylcarnitine; Asymmetric dimethylarginine; Sphingosine 1-phosphate; Sphinganine-1-phosphate (C18 base); Symmetric dimethylarginine; Taurine; Urea; Stearoylcarnitine; Eicosapentaenoic acid; 1-heptadecanoyl-2-hydroxy-sn-glycero-3-phosphocholine; Bilirubin; Biliverdin; Etiocholanolone glucuronide; Cotinine; Myristic acid; Stearic acid; L-(+)-Ergothioneine; Met_069 (unknown metabolite signal corresponding a molecular mass of 366); 1-Palmitoyl-2-hydroxy-sn-glycero-3-phosphocholine (LysoPC (16:0)); L-Acetylcarnitine; L-Lysine; L-Glutamine.

In one embodiment, the panel of obese pregnancy specific metabolite biomarkers” refers to at least 5, 10, 15 or substantially all of the metabolite biomarker selected from: biliverdin; Met_XXX; glycyl-glycine; NG-monomethyl-L-arginine; etiocholanolone glucuronide; eicosapentaenoic acid; dilinoleoyl glycerol; L-leucine; hexadecanoic acid; 25-hydroxyvitamin D3; linoleic acid; octadecenoic acid; stearoylcarnitine; stearic acid; 8, 11, 14 eicosatrienoic acid; 1-palmitoyl-2-hydroxy-sn-glycero-3-phosphocholine; L-isoleucine; bilirubin; L-arginine; L-(+)-ergothioneine; myristic acid; L-palmitoylcarnitine; arachidonic acid, urea; choline; taurine; docosahexaenoic acid; asymmetric dimethylarginine; L-methionine; 2-hydroxybutanoic acid; 3-hydroxybutanoic acid; L-acetylcarnitine; citrulline; decanoylcarnitine; dodecanoyl-I-carnitine; and sphingosine-1-phosphate.

In one embodiment the panel comprises at least 2, 3, 4, 5, 6, 7, 8, 9, 10, 11, 12, 13, 14 or 15 of the above obese pregnancy specific metabolite biomarkers. Typically, the panel comprises biliverdin or glycyl-glycine, and ideally comprises biliverdin and glycyl-glycine; Typically, the panel of obese pregnancy specific metabolite biomarkers comprises biliverdin; glycyl-glycine; NG-monomethyl-L-arginine; and dilinoleoyl glycerol. Typically, the panel of obese pregnancy specific metabolite biomarkers comprises biliverdin; glycyl-glycine; NG-monomethyl-L-arginine; dilinoleoyl glycerol; taurine, and stearic acid.

As used herein, the term “patient parameter for the pregnant obese woman” should be understood to mean a parameter selected from ethnicity, risk of gestational diabetes, fetal sex, number of pregnancies and level of obesity. “Ethnicity” refers to whether the patient is black or not. “Risk of gestational diabetes” refers to whether the obese pregnant woman has been determined to be at risk of gestational diabetes at the time of the assay (i.e. early in the pregnancy), or at low risk. Methods of identifying risk of gestational diabetes include the use of prognostic models that consist of simple predictors such as maternal age, body mass index, ethnicity, parity, history of GDM, and history of macrosomia¹⁻⁴ or a combination of maternal characteristics and maternal serum adiponectin and sex hormone-binding globulin at 11 to 13 weeks¹. “Fetal sex” refers to the sex of the foetus being carried by the pregnant obese woman-. Fetal sex can be established by ultrasound. Ultrasound has been the traditional method used for fetal sex determination. In the second and third trimesters, it is accurate in >99% of cases with normal genitalia.⁵ Early ultrasound (12-14 weeks) is also a reliable option when performed at specialized centers.⁶ Invasive testing, either using chorionic villus sampling from 11 weeks or amniocentesis from 15 weeks is also an option.⁷⁻⁸ More preferably, fetal sex can be determined by fetal sex determination using cell-free fetal DNA, for example using, but not limited to, DNA sequencing or PCR technology⁹⁻¹⁰. “Number of pregnancies” means determining whether the pregnancy is nulliparous (i.e. first pregnancy) or multiparous (second or subsequent pregnancy). “Level of obesity” means determining whether the pregnant obese woman is morbidly obese, i.e. has a BMI> or =40. As described herein, patient parameters can be used to pre-select one or more obese pregnancy metabolite biomarkers that are accurate predictors for risk of PE in the pregnant woman.

As used herein, the term “clinical risk factor” refers to one of the following:

| Parameter | Description |
|---|---|
| Mat_age | Maternal age |
| Mat_age_GE35 | Maternal age greater than or equal to 35 years |
| Mat_age_LT25 | Maternal age lower than 25 years |
| Mat_chol | Maternal cholesterol at first visit |
| Mat_hdl | Maternal HDL at first visit |
| Mat_ldl | Maternal LDL at first visit |
| Mat_ldl_hdl | Maternal HDL/HDL at first visit |
| Mat_trig | Maternal triglycerides at first visit |
| Mat_bmi | BMI at first visit |
| Mat_prev_pet | Pre-eclampsia in any previous pregnancy |
| fh_any_pet | Family history of Pre-eclampsia (Mother of |
| | pregnant woman, sister of pregnant woman) |
| Mat_dbp_m (BP) | Maternal diastolic blood pressure at baseline, |
| | calculated from duplicate measurements |
| Mat_sbp_m (BP) | Maternal systolic blood pressure at baseline, |
| | calculated from duplicate measurements |
| gaw_1st_visit | Gestation at first visit in weeks |
| uncomp_pregn_score | Multivariable Risk score to predict an |
| | uncomplicated pregnancy in obese pregnant |
| | women ⁴⁷ |
| MAP | Mean Arterial Blood pressure |

As used herein, the term “blood pressure” may be maternal diastolic or maternal systolic blood pressure, or mean arterial pressure (MAP) which is estimated as follows: MAP=[⅔ (diastolic bp)+⅓ (systolic bp)].

As used herein, the term “patient parameter specific clinical risk factor” means a clinical risk factor that is useful in early prediction of PE is a pregnant obese sub-group that exhibits the patient parameter. An example is blood pressure is pregnant obese women carrying a male offspring.

As used herein, the term “computational model” refers to a computer processor configured to receive as an input at least the abundance values for one or more metabolites, perform univariable or multivariable analysis on the input value(s), and provide an output comprising a quantitative estimate of pre-eclampsia risk.

The risk that a pregnant individual develops pre-eclampsia can be determined from metabolite concentrations, and other variables, like clinical risk factors and/or protein abundances using statistical analysis based on data collected in a representative patient population study. Examples 2 to 4 show results from such studies. There are multiple statistical methods for combining parameters that characterize the pregnant individual, such as abundance of metabolite markers, to obtain a risk estimate. The likelihood method 48 and the linear discriminant function method 49 are commonly used for this purpose. The basic principle of the likelihood method is that the population distributions for a variable (such as the abundance of a metabolite marker) or the risk scores as obtained by the application of a multivariable algorithm are known for the ‘unaffected’ (“controls”) and ‘affected’ groups (“cases”).

For univariable analysis, the computational model is configured to compare the input value with the likelihood of membership of the ‘non pre-eclampsia destined” and ‘pre-eclampsia destined’ groups. From this, the computational model is determining a subject's risk for pre-eclampsia, and the metabolite concentration is correlated to a likelihood of pre-eclampsia. For example, the measured concentrations may be compared to a threshold value.

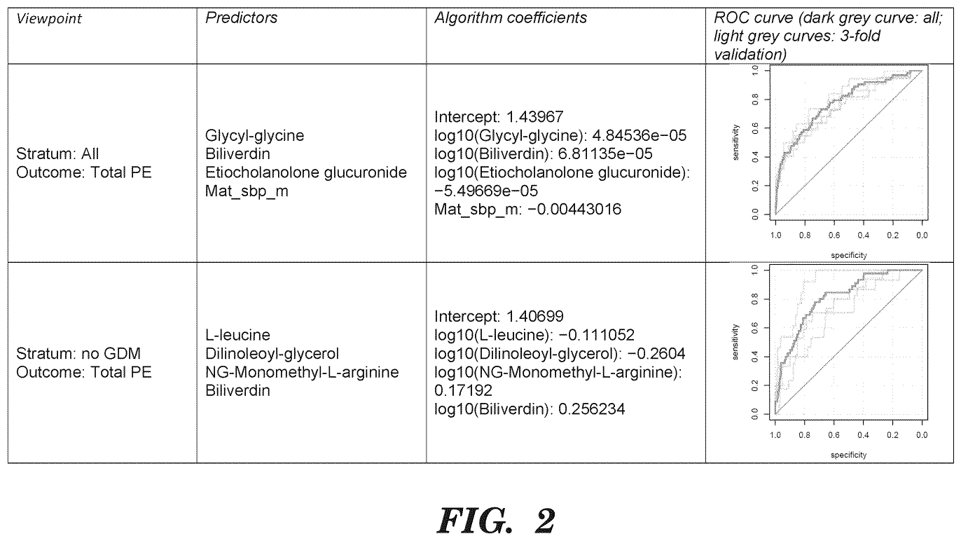

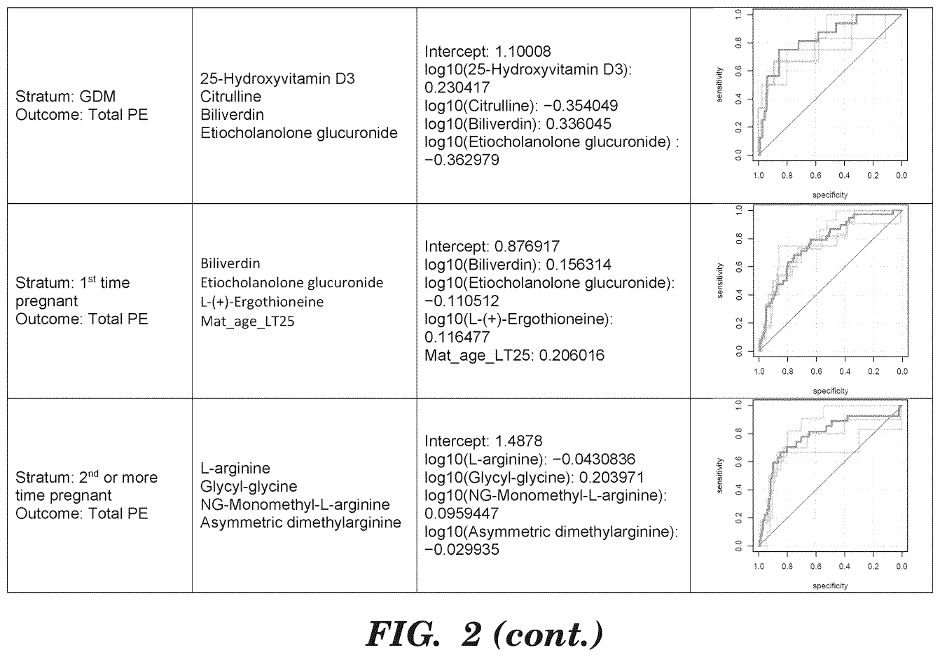

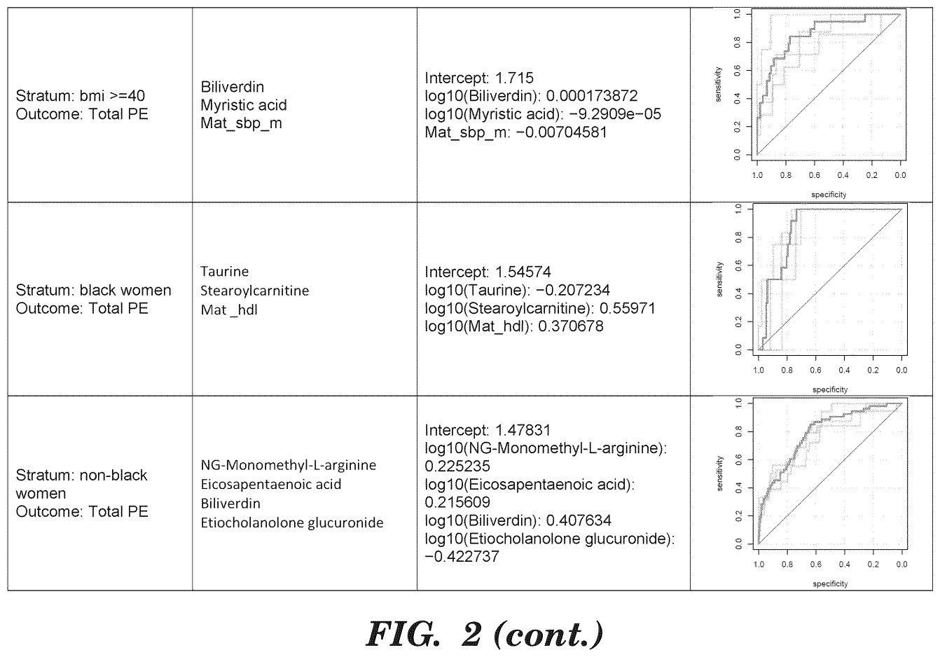

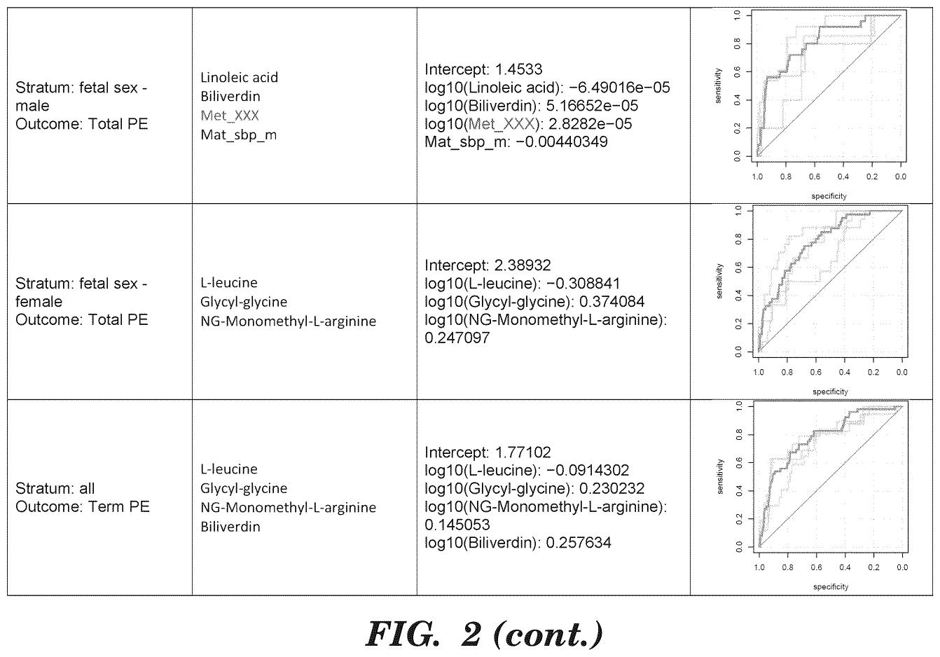

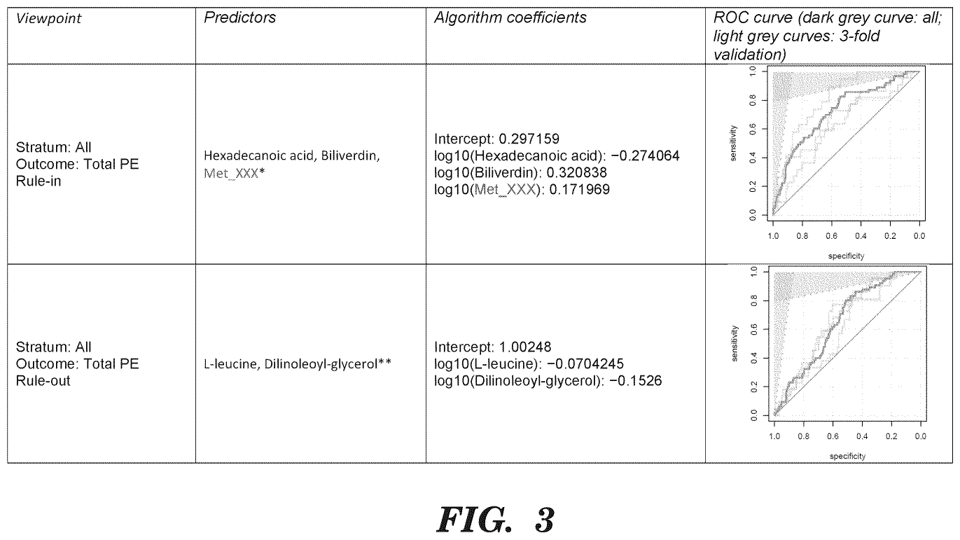

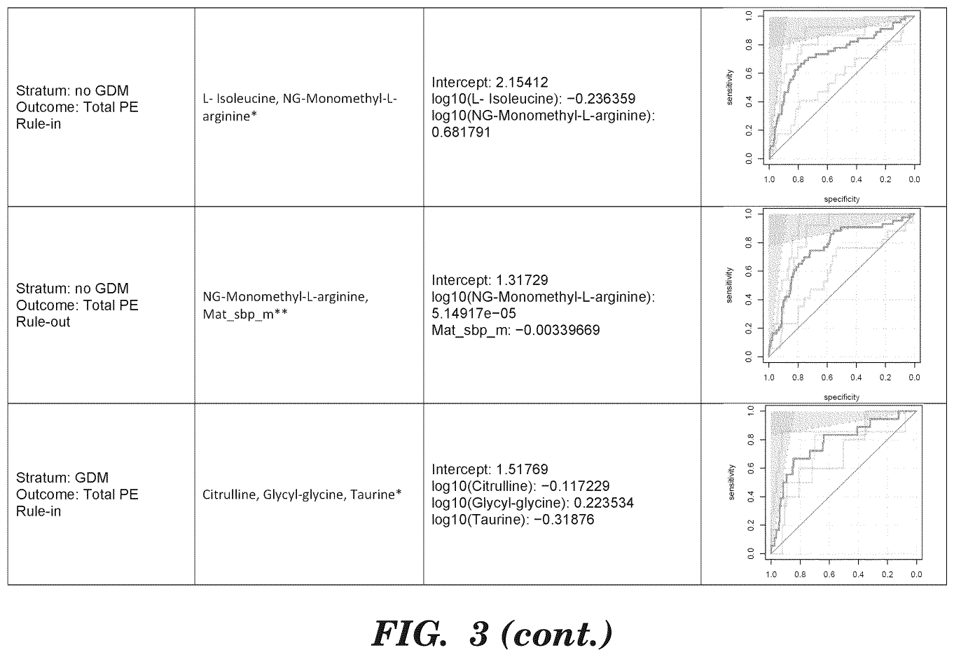

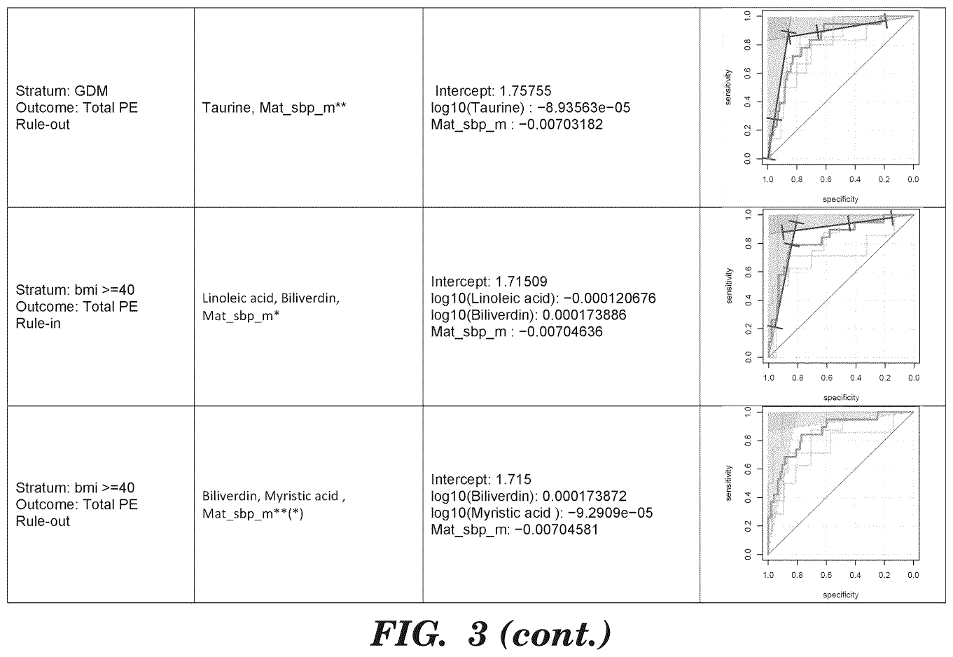

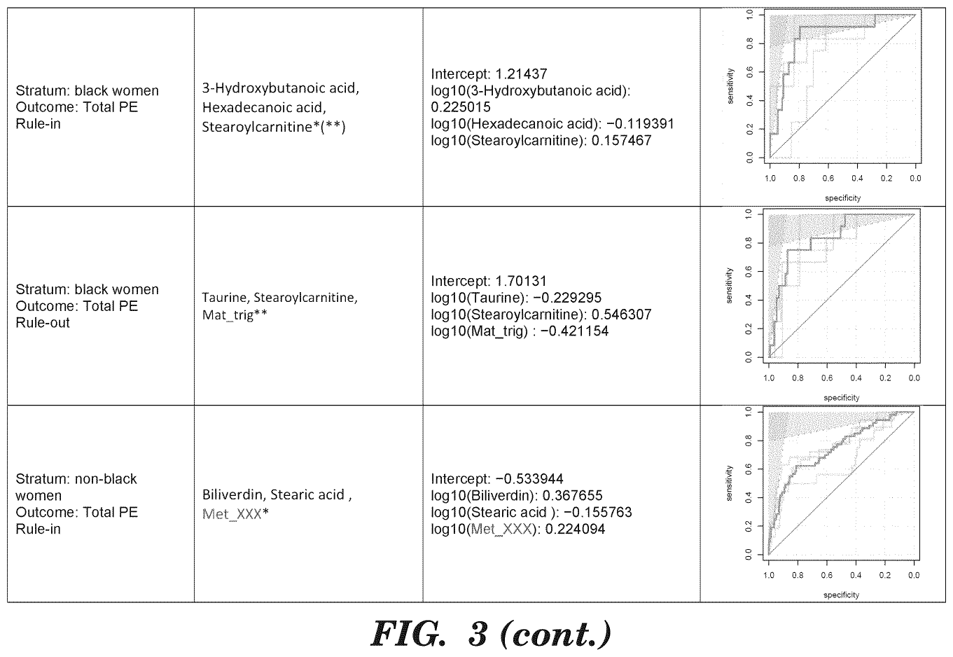

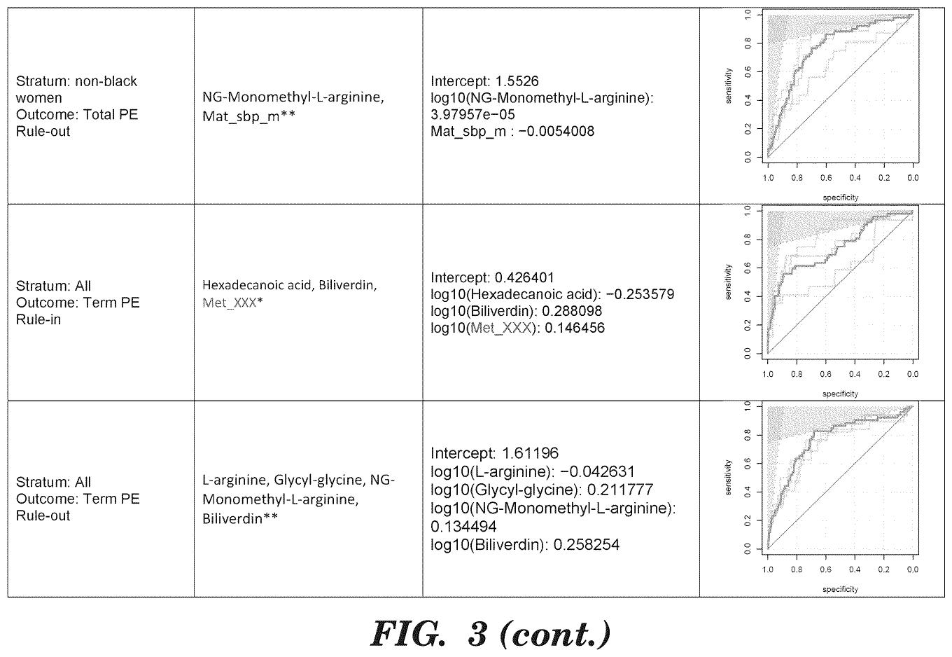

For multivariable analysis, the model generally employs an algorithm—examples of suitable algorithms are provided in the FIGS. 2 and 3 below. In one embodiment, the computational model is configured to (a) combine the abundance values of the plurality of metabolites (and optionally one or more protein biomarkers and/or clinical risk factors) into a risk score using a multivariable algorithm, (b) compare the risk score with the likelihood of membership of the ‘non pre-eclampsia destined” and ‘pre-eclampsia destined’ groups—from this, the computational model determines a subject's risk for pre-eclampsia, and the risk score is correlated to a likelihood of pre-eclampsia developing (for example, the calculated risk score may be compared to a threshold value) and (c) output a predicted risk of pre-eclampsia based on the comparison.

In some embodiments, the computational model is configured to select a subset comprising at least one, and generally two, of the panel of obese pregnancy specific metabolite biomarkers based on the patient parameter input, and correlate abundance values for the subset of metabolites with risk of PE.

As used herein, the term “modulated abundance” as applied to a metabolite biomarker means an abundance of the metabolite that is either increased or decreased in a test sample compared with a reference abundance for the specific metabolite. Tables 8-14 (ANNEX) indicates, for each metabolite, whether the metabolite-on average-exhibits increased or decreased abundance in a population of pregnant obese women developing pre-eclampsia compared with a reference abundance for the metabolite as calculated from the metabolite abundances in a population of pregnant obese women not developing pre-eclampsia. Thus, for example, detection of increased abundance of Dilinoleoyl-glycerol or Etiocholanolone glucuronide in a pregnant obese woman relative to a reference abundance correlates with risk of the pregnant woman obese woman developing pre-eclampsia. Likewise, detection of decreased abundance of NG-Monomethyl-L-arginine, or Biliverdin, Glycyl-glycine in a pregnant obese woman relative to a reference abundance correlates with risk of the pregnant woman obese woman developing pre-eclampsia. For any of the metabolite markers, methods of detection of modulated abundance are described below. Thus, for example, when (UP) LC-MS is employed as means of detecting modulated abundance, the critical p-value for clinical significance is ideally at most 0.05.

As used herein the term “reference abundance” as applied to a metabolite biomarker should be understood to mean the mean abundance (or median abundance) of the metabolite marker as calculated from the metabolite abundances in the biological samples from a population of pregnant obese women not developing pre-eclampsia. The population of pregnant obese women not developing pre-eclampsia may be obese pregnant women with a pregnancy without pre-eclampsia, or a pregnancy without pre-eclampsia and without any other complications. Ideally, the population is age matched and the biological samples are ideally obtained from subjects at the same gestational period as the test subject +/−4 weeks, 3 weeks, 2 weeks, or 1 week. In another embodiment, the reference abundance may be calculated from the metabolite abundances in the biological samples from a population of pregnant obese women who subsequently develop pre-eclampsia, the biological samples being ideally obtained from subjects at the same gestational period as the test subject +/−4 weeks, +/−3 weeks, +/−2 weeks, or +/−1 week. In this case, modulated abundance would predict, or aid in the prediction, of non-occurrence of pre-eclampsia (i.e. a normotensive pregnancy).

As used herein, the term “prophylactic therapy” refers to a therapeutic intervention for pregnant obese women to prevent development of pre-eclampsia typically during the second or third trimester of pregnancy. Examples of therapeutic intervention include Aspirin; Low Molecular Weight Heparin; Restriction of weight gain by either caloric intake reduction or life style changes; Interventions to lower the glycemic index, including but not restricted to, insulin, glycemic index lowering probiotics; Citrulline; Antioxidants, including but not limited to, antioxidant vitamins (e.g., ascorbic acid, -tocopherol, -carotene), inorganic antioxidants (e.g., selenium), and a plant-derived polyphenols, antioxidants to mitochondria, including but not limited to, Mito VitE and ergothioneine; statins, including but not limited to, Pravastin. Therapies involving the use of anti-inflammatory or immunosuppressive agents like, but not limited to, tacrolimus, or sulfalazine. In addition, one can easily envision preferred therapeutic combinations like, but not limited to, aspirin and metformin; or metformin and sulfalazine.

As used herein, the term “metformin treatment” refers to treatment with Metformin (GLUCOPHAGE) or a combination therapy comprising Metformin and an addition drug, for example aspirin, thiazolidinediones, DPP-4 inhibitors, sulfonylureas, and meglitinide.

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. CD ROM, or magnetic recording medium, e.g. a floppy disk or hard disk. The carrier may be an electrical or optical signal which may be transmitted via an electrical or an optical cable or by radio or other means.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1—Methods

### Participants and Specimens

Prospective clinical samples were collected from pregnant women with a singleton pregnancy at first visit (15+0 to 18+6 weeks) weeks' gestation) and which were either diagnosed with pre-eclampsia (cases) or not diagnosed with pre-eclampsia (controls) in the further course of their pregnancy. All samples were obtained from participants in the UPBEAT (the UK Better Eating and Activity Trial) prospective screening study of nulliparous women⁵⁰.

Written consent was obtained from each participant. The inclusion criteria applied for the study were women with a BMI>30 kg/m2 and a singleton pregnancy between 15+0 weeks and 18+6 weeks' gestation. The exclusion criteria applied were: women unable or unwilling to give informed consent; <15+0 weeks or >18+6 weeks' gestation; essential hypertension requiring treatment either pre-pregnancy or in index pregnancy; pre-existing renal disease; systemic lupus erythematosus; antiphospholipid syndrome; sickle cell disease; thalassemia; coeliac disease; thyroid disease; current psychosis; multiple pregnancy; currently prescribed metformin.

Pre-eclampsia was defined according to the International Society for the study of Hypertension in Pregnancy (ISSHP) criteria: two measures of systolic blood pressure (BP)>=140 mmHg and/or diastolic BP ≥=90 mmHg on at least 2 occasions 4 hours apart after 20 weeks gestation and the presence of proteinuria >=300 mg/24 h or >=1 g/l, or spot urine protein: creatinine ratio >=30 mg/mmol creatinine or urine dipstick protein >=212.

Note that, in the cohort studied, 22% of the pregnant participants would go on to develop gestational diabetes mellitus (GDM) later in their pregnancy.

### Pre-Eclampsia Rate in Function of Bmi in UPBEAT

Within the UPBEAT cohort, the bmi distribution across the various bmi classes is as follows: 49.2% of the population in class I obesity (BMI 30-34.9); 37.7% in class II obesity, (BMI 35-39.5); and 18.1% in class III obesity (BMI>40). The overall rate of pre-eclampsia increased over the respective obesity classes, with 4.4% in class I obesity, 6.5% in class II obesity and 8.9% in class III obesity.

### Sample Set Used

A nested case-control study was conducted within the UPBEAT cohort, using blood samples taken at 15+0-18+6 weeks of gestation; the cohort constituted a case: control ratio of 1:10. Cases are defined as these pregnant women who develop pre-eclampsia (as defined earlier) in the course of their pregnancy. For the avoidance of doubt, at time of blood sampling none of the women showed any signs of PE, what's more in accordance to the clinical definition of PE, PE can only manifest from 20 weeks of gestation onward. Within the study 65 cases were considered, this corresponds all cases within UPBEAT for which samples were available. Controls were randomly selected amongst all other pregnancies. To avoid artefacts due to selection bias, the demographic and clinical characteristics of the control population selected to the study was compared and the lack of bias was verified using the appropriate statistical tests; the Chi-squared, Spearman Correlation, Mann Whitney U and Kruskal-Wallis tests were used as appropriate. Samples of 647 controls pregnancies were selected to the study.

In Table 1, the baseline characteristics of this study cohort are presented. Using this study cohort the applicants engaged in discovering univariable and multi-variable predictors for the prediction of pre-eclampsia in the obese pregnant women.

TABLE 1
Characteristics of the study population (Stratum: all; Outcome: Total PE)

| | Control (non PE destined) | Case (PE destined) |
|---|---|---|
| Count | 645 | 65 |
| Obesity | class I obesity: 318 (49%) | class I obesity: 25 (38%) |
| | class II obesity: 216 (33%) | class II obesity: 20 (31%) |
| | class III obesity: 111 (17%) | class III obesity: 20 (31%) |
| BMI | 35.1 (32.8-38.4) | 37.4 (33.1-41.3) ° |
| Mother's age | 30.4 (5.49) | 30.4 (4.99) |
| Ethnicity | Asian: 32 (5%) | Asian: 4 (6.2%) |
| | Black: 132 (20%) | Black: 12 (18%) |
| | Other: 35 (5.4%) | Other: 5 (7.7%) |
| | White: 446 (69%) | White: 44 (68%) |
| Centres | St Thomas': 232 (36%) | St Thomas': 15 (23%) |
| | Newcastle: 123 (19%) | Newcastle: 22 (34%) |
| | Glasgow: 152 (24%) | Glasgow: 12 (18%) |
| | Manchester: 80 (12%) | Manchester: 6 (9.2%) |
| | Bradford: 16 (2.5%) | Bradford: 4 (6.2%) |
| | St Georges': 42 (6.5%) | St Georges': 6 (9.2%) ° |
| Gestational age at delivery | 39.9 (38.7-41) | 38.7 (37.7-39.7) *** |
| Delivery | | |
| extreme preterm: GA < 34 wks | extreme preterm: 16 (2.5%) | extreme preterm: 6 (9.2%) |
| GA | preterm: 38 (5.9%) | preterm: 13 (20%) |
| preterm: GA < 37 wks GA | term: 606 (94.1%) | term: 52 (80%) ** |
| term: GA >= 37 wks GA | | |
| Systolic BP at first visit | 119 (10.8) | 126 (11.8) *** |
| Diastolic BP at first visit | 72.6 (7.69) | 77.7 (7.98) *** |
| multiparous | FALSE: 287 (44%) | FALSE: 38 (58%) |
| | TRUE: 358 (56%) | TRUE: 27 (42%) ° |
| Aspirin treatment | FALSE: 552 (86%) | FALSE: 48 (74%) |
| | TRUE: 93 (14%) | TRUE: 17 (26%) ° |
| Statins treatment | FALSE: 645 (100%) | FALSE: 65 (100%) |
| Gestation age at first visit | 16 (1.05) | 15.8 (1) |
| Previous PET | FALSE: 338 (93%) | FALSE: 21(72%) |
| | TRUE: 24 (6.6%) | TRUE: 8 (28%) ** |

Results are expressed as mean (SD), median (interquartile range), or n (%).
° p < 0.05,
* p < 0.01,
** p < 0.001,
*** p < 10e−04 future cases vs controls, Chi-squared test, T test or Mann Whitney U test.

The applicants realised that even within the obese pregnancy population, there may still exist different risk profiles in different sub-populations; likewise, with pre-eclampsia being a syndrome, different sub-outcomes may also overlap. Therefore, to comprehensively encapsulate the complexity of predicting of PE in the obese pregnant population, prognosis of PE was also considered in several population sub-groups (strata) and outcomes. In Table 2, the different viewpoints taken to discover additional predictors, and non-trivial combinations thereof, which would not be discoverable when considering the pregnant obese as a single homogeneous group (Stratum: “all”) and/or pre-eclampsia as a single disease entity (Outcome: “Total PE”).
In view of the significant fraction of women developing GDM, pre-eclampsia prognosis was therefore investigated in the subsets of women with and without a gestational diabetes mellitus pregnancy outcome.
With first time pregnant being considered a pregnancy population with increased risk, differentiation between 1^{st} time obese pregnant and obese women who had been pregnant before was also looked into.
People having a bmi >=40, are considered to be morbidly obese, putting them at high risk of developing obesity-related health conditions; identifying within the group of morbidly obese pregnant women, the women at increased risk of pre-eclampsia may inform the prenatal care of this vulnerable group.
Ethnic origins have been associated with different risk profiles for pre-eclampsia, with black women often having a higher risk of developing pre-eclampsia. 51. The applicants looked into discovering predictors, and non-trivial combinations thereof, specific to prediction of pre-eclampsia in obese black women (vs. obese non-black women).
Sex of the fetus may also influence how pre-eclampsia develops, hence the applications considered looked into discovering predictors, and non-trivial combinations thereof, specific to predicting pre-eclampsia in obese women carrying either a female or male child.
Lastly, it is well established that preterm pre-eclampsia, i.e., pre-eclampsia warranting for the delivery of the baby before 37 weeks of gestation, may develop differently than term pre-eclampsia, hence the applicants looked into predicting preterm and term pre-eclampsia separately in obese women.

Additional prognostic viewpoints considered for the prognosis of PE in the obese pregnancy population are summarised in Table 2:

| Population sub-group/pre-eclampsia sub-outcome | Cases | Controls |
|---|---|---|
| (italic) | (N=) | (N=) |
| Prognosis of PE in the obese pregnant who don't | 45 | 465 |
| develop the GDM co-morbidity | | |
| Prognosis of PE in the obese pregnant who do | 18 | 169 |
| develop the GDM co-morbidity | | |
| Prognosis of PE in the first-time pregnant women | 38 | 287 |
| Prognosis of PE in the non-first-time pregnant | 27 | 358 |
| women, i.e.,within women who have been pregnant | | |
| before | | |
| Prognosis of PE in the morbidly obese pregnant | 20 | 111 |
| women, i.e., women with a bmi >= 40 | | |
| Prognosis of PE in pregnant women of black | 12 | 132 |
| ethnicity | | |
| Prognosis of PE in pregnant women of non-black | 53 | 513 |
| ethnicity | | |
| Prognosis of PE in pregnant women with male | 25 | 328 |
| offspring | | |
| Prognosis of PE in pregnant women with female | 40 | 315 |
| offspring | | |
| Prognosis of preterm PE. i.e., PE warranting a | 13 | 645 |
| delivery before 37 weeks of gestation | | |
| Prognosis of term PE. i.e., PE not leading to a | 52 | 645 |
| delivery before 37 weeks of gestation | | |

### Targeted Quantification of Metabolites.

The following describes one exemplary and preferred way of targeted quantification of metabolites in samples, particularly as also used in the present examples.

### Metabolites of Interest:

Table 3 (Annex) tabulates a non-limiting list of metabolites of interest which are considered in this application. These metabolites, and or metabolite classes, are deemed relevant by the inventors in view of identifying non-obvious prognostic combinations of metabolites, to predict risk of pre-eclampsia in a pregnant woman prior to appearance of clinical symptoms of pre-eclampsia in the woman pre-eclampsia. Where possible the metabolites of interest are identified by their CAS number, or/and their HMDB identifier; the molecular weights are also given (na: not available).

### The Analytical Methods are Based on the Following:

A. The use of an extraction solvent/protein precipitation solvent that enables the extraction of the different types (classes) of metabolites:

This extraction solvent composition, being a mixture of Methanol, Isopropanol and 200 mM Ammonium Acetate (aqueous) in a 10:9:1 ratio, which in turn is fortified with 0.05% 3,5-Di-tert-4-butyl-hydroxytoluene. In the remainder of this example this solvent is referred to as the “crash”.

B. The use of a dual (High Pressure) Liquid Chromatography (LC) system to enable the identification and quantification of the different classes of metabolites in a short analytical run.

The chromatographic systems were developed so that these could be directly hyphenated to a mass spectrometric detection system. This dual chromatography system allows the separation of different metabolite types/classes and at the same time generate a detectable signal at the level of the mass spectrometer. A single chromatographic system, with short turn-around time, is not effective in robustly generating a detectable signal across all classes. The ability to 1) comprehensively analyse metabolites across different classes of metabolites, as relevant to a prognostic question, in 2) a short turn-around time, is important to generate data on sufficiently large sample sets (necessary to enable statistically robust multivariable models) in economically viable time- and cost-frames.

Typical, but non-limiting examples of LC methods, are detailed below:

### Materials and Reagents Used in the Dual Separations:

LC-MS grade ammonium acetate (NH4OAc) and ammonium formate (NH4HCOO) were purchased from Fluka (Arklow, Ireland). LC-MS optima grade acetic acid, acetonitrile (ACN), methanol (MeOH) and 2-Propanol (IPA) were purchased from Fischer scientific (Blanchardstown, Ireland).

For the RPLC, the column-type used was a Zorbax Eclipse Plus C18 Rapid Resolution HD 2.1×50 mm, 1.8-Micron column (P.N. 959757-902; Agilent Technologies, Little Island, Ireland). For the HILIC-MS/MS, the column type was an Ascentis Express HILIC 15 cm×2.1 mm, 2.7 Micron (P.N. 53946-U: Sigma-Aldrich, Arklow, Ireland)

### Instrument:

The LC-MS/MS platform used consisted of a 1260 Infinity LC system (Agilent Technologies, Waldbronn, Germany). The latter was coupled to an Agilent Triple Quadrupole 6460 mass spectrometer (QqQ-MS) equipped with a JetStream Electrospray Ionisation source (Agilent Technologies, Santa Clara, CA, USA).

### RPLC:

The RPLC method is defined by the following settings/parameters:
Injection volume: 7 μL
Column oven temperature: 60° C.
Gradient RPLC was performed to resolve the hydrophobic metabolites using a binary solvent system:
   mobile phase A: Water:MeOH:NH4OAc buffer 200 mM at pH 4.5, (92:3:5)
   mobile phase B: MeOH:ACN:IPA:NH4OAc 200 mM at pH 4.5 (35:35:25:5)
A linear gradient program was applied: from 10% mobile phase B to 100% mobile phase B in 10 minutes. using the following gradient-flow rate program:

| Time | % Mobile | % Mobile | Flow rate |
|---|---|---|---|
| (min) | phase A | phase B | (ml/min) |
| 0.00 | 100% | 0% | 0.35 |
| 6.00 | 0% | 100% | 0.5 |
| 8.00 | 0% | 100% | 0.5 |
| 8.10 | 100% | 0% | 0.5 |
| 9.00 | 100% | 0% | 0.5 |
| 10.00 | 100% | 0% | 0.35 |

The efflux of the RPLC column was led directly to the QqQ-MS for mass spectrometric determination of the hydrophobic compounds of interest (see below)).

### HILIC:

The HILIC method is defined by the following settings/parameters:
Injection volume: 3 μL, whereby the injection plug was bracketed by 3 μL ACN solvent plugs; a specific injector program was devised for this.
Column oven temperature: 30° C.
Gradient HILIC was performed to resolve the hydrophobic metabolites using a binary solvent system:
   mobile phase A: 50 mM Ammonium formate (aqueous)
   mobile phase B: ACN
A linear step gradient program was applied: from 10% mobile phase B to 100% mobile phase B in 10 minutes. using the following gradient-flow rate program:

| Time | % Mobile | % Mobile | Flow rate |
|---|---|---|---|
| (min) | phase A | phase B | (ml/min) |
| 0.00 | 12% | 88% | 0.45 |
| 1.10 | 20% | 80% | 0.45 |
| 2.0 | 20% | 80% | 0.45 |
| 2.10 | 30% | 70% | 0.45 |
| 3.00 | 30% | 70% | 0.45 |
| 3.10 | 40% | 60% | 0.45 |
| 4.00 | 40% | 60% | 0.45 |
| 6.00 | 50% | 50% | 0.45 |
| 7.20 | 50% | 50% | 0.45 |
| 7.21 | 12% | 88% | 0.45 |
| 10.00 | 12% | 88% | 0.45 |

The efflux of the RPLC column was led directly to the QqQ-MS for mass spectrometric determination of the hydrophobic compounds of interest (see below)).

C. The use of a form of quantitative mass spectrometry, i.e., a tandem mass spectrometry system (MS/MS) operated in the Multiple Reaction Monitoring modus to allow for sensitive and specific analysis of metabolites.

Hereto the samples are subjected to ionization under conditions to produce ionized forms of the metabolites of interest. Then the ionized metabolites are fragmented into metabolite derived fragment ions. The amounts of two specific fragments per metabolite are determined to identify and quantify the amounts of the originator metabolites in the sample (for further detail see below). Tandem mass spectroscopy was carried out under both positive and negative electrospray ionization and multiple reaction monitoring (MRM) mode. For each metabolite of interest, as relevant to pre-eclampsia, the following parameters were specifically established and optimized for each and every metabolite of interest and each SIL-IS available:
appropriate precursor ion m/z, inclusive its preferred ionization mode (positive or negative),
Product ion spectra under various collision voltage conditions (cf. induction of ion-molecule collisions under different energy regimens, leading to specific product ions) and selection of the most appropriate Quantifier and Qualifier product ions to be used for mass spectrometric identification and quantifications.
Establishment of the reference Quantifier ion/Qualification ion ratios which to serve for specificity assessment.
In addition, a number of assay specific instrument parameters were also optimized per compound of interest: quadrupole resolutions, dwell time, Fragmentor Voltage, Collision Energy and Cell Accelerator Voltage.
At the same time, instrument-specific parameters were optimised to maximally maintain compound integrity in the electrospray source and achieve sensitive and specific metabolite analysis; source temperature, sheath gas flow, drying gas flow and capillary voltage. The mass spectrometer used was an Agilent Triple Quadrupole 6460 mass spectrometer (QqQ-MS) equipped with a JetStream Electrospray Ionisation source (Agilent Technologies, Santa Clara, CA, USA).

### RPLC-ESI-MS/MS

For the mass spectrometric method used for analyzing the hydrophobic metabolites of interest, the optimized electrospray ionization source parameters were as follows:

| Source Parameters | Positive mode | Negative mode |
|---|---|---|
| Gas Temperature, ° C. | 200 | 200 |
| Gas flow, l/min | 13 | 13 |
| Nebuliser, psi | 40 | 40 |
| Sheath Gas Heater | 400 | 400 |
| Sheath Gas Flow | 11 | 11 |
| Capillary, V | 5000 | 3000 |
| V Charging | 300 | 300 |

### HILIC-MS/MS:

For the mass spectrometric method used for analyzing the hydrophilic metabolites of interest, the optimized electrospray ionization source parameters were as follows:

| Parameters | Positive mode | Negative mode |
|---|---|---|
| Gas Temperature, ° C. | 200 | 200 |
| Gas flow, l/min | 13 | 13 |
| Nebuliser, psi | 40 | 40 |
| Sheath Gas Heater | 400 | 400 |
| Sheath Gas Flow | 12 | 12 |
| Capillary, V | 2500 | 3000 |
| V Charging | 300 | 300 |

D. For each metabolite a specific LC-MS/MS assay was developed for each of the targets of interest as well as for each of the SIL-IS;

A particular LC-MS/MS assay entails a combination of above points 2&3. To unambiguously identify a metabolite/SIL-IS of interest, each of the assays will constitute a specific set of experimental parameters which will unequivocally identify the compound of interest.

It is of note that the values of these experimental parameters are specific to and optimized for the used LC-MS/MS technology. In the case of the LC-MS/MS assays under consideration, this set of specific parameters are the following:
a) Retention time (Rt): The time between the injection and the appearance of the peak maximum (at the detector). The specific retention time is established for each metabolite.
b) Precursor ion m/z: Mass/charge ratio of the ion that is directly derived from the target compound by a charging process occurring in the ionisation source of the mass spectrometer. In this work the precursor ion is most often a protonated [M+H]⁺ or deprotonated form [M−H]⁻ of the target compound. In some instances, the precursor ion considered has undergone an additional loss of a neutral entity (f.i., a water molecule (H₂O)) in the ionisation source. In some other instances, the ionisation of the compound of interest follows the formation of an adduct between the neutral compound and another ion (f.i, a sodium adduct) available. The appropriate precursor ion is established for each metabolite.
c) Precursor ion charge: The charge of the ion that is directly derived from the target compound by a charging process occurring in the ionisation source of the mass spectrometer, the precursor ion can be either positively charged or negatively charged. The appropriate charge state is established for each metabolite.
d) Quantifier Product ion: Ion formed as the product of a reaction involving a particular precursor ion. The reaction can be of different types including unimolecular dissociation to form fragment ions, an ion-molecule collision, an ion-molecule reaction 53, or simply involve a change in the number of charges. In general, the quantifier product ion is the most intense fragment and/or specific to the compound of interest. The quantifier product ion data is used to quantify the compound of interest. The appropriate quantifier product ion is established for each metabolite and SIL-IS.
e) Qualifier Product ion: Ion formed as the product of a reaction involving a particular precursor ion. The reaction can be of different types including unimolecular dissociation to form fragment ions, an ion-molecule collision, an ion-molecule reaction 53, or simply involve a change in the number of charges. In general, the qualifier product ion is a less intense fragment to the compound of interest. The qualifier product ion data is used as an additional confirmation the LC-MS/MS is specific to the compound of interest. In specific cases, the use of more than one qualifier ions is considered. The appropriate qualifier product ion is established for each metabolite and SIL-IS.
f) Quantifier ion/Qualifier ion ratio (or vice versa): under well-defined tandem mass spectrometric conditions, a precursor ion produced from a compound of interest will dissociate in controlled fashion and generate quantifier product ions and qualifier product ions in predictable proportions. By monitoring the quantifier/Qualifier ratio, one gets additional assurance that the LC-MS/MS is specifically quantifying the compound of interest. The chance that an interference will elute at the same retention time, create the same precursor ion, and dissociate in the same quantifier and qualifier ions in the same proportion as the target of interest is deemed very low. In specific cases, the use of more than one quantifier/Qualifier ratio can be considered. The appropriate Quantifier ion/qualifier ion ratio (or vice versa) is established for each metabolite and SIL-IS.

Availability of the above 6 parameters will define with great certainty a highly specific assay to a compound of interest. In some instances, not all 6 parameters will be available, f.i., when the precursor ion will not dissociate in meaningful product ions.

For these metabolite targets wherefore a structurally identical SIL-IS standard is co-analyzed, one has an additional specificity metric: the metabolite target and the SIL-IS are, apart from their mass, chemically identical, and hence they would have the same retention time. In rare instances, perfect co-elution is not achieved due to a so-called deuterium effect 54.

The specific parameter sets established for exemplary metabolites and associated SIL-IS across the metabolite classes of interest to the prediction of pre-eclampsia, together with some instrument specific (but non-limiting) settings are presented in Tables 4&5 (Annex). For the un-identified Met_XXX, these parameters will unequivocally define the metabolite signal.

### E. The use of Stable Isotope Labelled Internal Standards (SIL-IS)

The use of SIL-IS enable Stable Isotope dilution mass spectrometry. Herewith one can achieve precise and accurate mass spectrometry-bases compound quantifications 5556. In brief, Stable Isotope Dilution Mass spectrometry is based on the principle that one fortifies, at the start of the analytical process, all study samples with the same volume of a well-defined mixture of SIL-IS. These SIL-IS are typically identical to the endogenous compounds of interest, in this case metabolites, but have a number of specific atoms (typically Hydrogen 1H, Nitrogen 14N or Carbon 12C) within their molecular structure replaced by a stable, heavy isotope of the same element (typically Deuterium 2H, Nitrogen 15N, Carbon 13C). The SIL-IS are therefore chemically identical but have a different “heavier” mass than their endogenous counterparts. Since they are chemically identical, they will “experience” the same experimental variability as the endogenous metabolites of interest. For instance, any differential extraction yield between study samples during sample preparation will equally affect the metabolite of interest and its corresponding SIL-IS. Equally, the metabolite of interest and its corresponding SIL-IS will undergo the same chromatography and are typically equally sensitive to variability during mass spectrometric analysis. As a result, the ratio of any target metabolite signal and its according SIL-IS signal are largely invariant to experimental variability, hence the ratio “metabolite signal/corresponding SIL-IS signal” is directly related to the original concentration of the target in the blood sample. So, in the here disclosed methods, the preferred way to precisely quantify the amount of a metabolite of interest in a sample is by means of establishing the ratio of “the amount of the target metabolite quantifier ion/the amount of the quantifier ion of the corresponding SIL-IS”. Whereby the here disclosed methods allow one to quantify a multitude of different target metabolites in a single analysis of the sample. Moreover, as all study samples are fortified with the same volume of a well-defined mixture of SIL-IS, one can readily compare the levels of the metabolites of interest across all study samples. The SIL-IS are exogenous compounds and thus not to be found in the native biological samples, so their spiked levels act as a common reference for all study samples.

F. The use of specific sample processing protocols for the simultaneous processing of large batches of biospecimens with high reproducibility and low technical variability.

The details of a non-limiting example of a fit-for-purpose processing protocol is elaborated below.

As part of the methods, a dedicated biospecimen preparation methodology has been established, involving the fortification of the samples with a relevant SIL-IS mixture, and the use of the “crash” to extract the metabolites of interest. In terms of sample handling, minimizing any potential sources of error is critical to ensure reliable and precise results. The critical source of error in this methodology relates to the control of volumes: the most critical volumes are the actual specimen volume available for analysis, and the volume of the SIL-IS added. Whereas experienced lab analysts can prepare samples precisely, the use of robot liquid handlers is preferred when processing large numbers of biospecimens as their pipetting accuracy and imprecision are more controllable, whereas human induced technical variability may be more unpredictable in nature.

Here, as a non-limiting example, we elaborate a dedicated blood processing process, as relevant to methods in this application, using a liquid handling robot.

The robot was configured to enable 96 blood specimens in parallel, using the well-established 96 well format; this is also the analytical batch format adopted for the collection of methods herein.

### Instrument:

Agilent Bravo Automated Liquid Handling Platform (BRAVO, Model 16050-102, Agilent Technologies, Santa Clara, CA, USA), equipped with, a 96 LT disposable Tip Head, an orbital shaker station and a Peltier Thermal Station (Agilent Technologies). The Robot deck has 9 predefined stations, which can be used for 96 well-plates (specimens, reagents, pipette tip boxes) or functional stations (e.g. Peltier Station, etc).

### Experimental Protocol:

In brief the following steps were performed for each batch of 96 40 μl aliquots; partial batches (n<96) are processed identically:
a) A 96-position plate (8×12 positions, PN: W000059X, Wilmut, Barcelona, Spain) with pre-ordered and 40 μl pre-aliquoted specimens (0.65 ml cryovials, PN: W2DST, Wilmut, Barcelona, Spain), constituting an analytical batch, are retrieved from −80° C. storage, and put on BRAVO deck (orbital shaker) and vortexed for 20 minutes to assist thawing. When thawed, the vials are decapped (manually).
b) In the meantime,
   a. a pre-prepared SIL-IS aliquot is retrieved from −20° C. storage for thermal conditioning, the SIL-IS is then vortexed (1 minute) and-sonicated (5 minutes), and the appropriate volumes are then placed in one column (8 wells) of a PolyPropylene (PP) 96 well plate. The SIL-IS plate is then placed on the BRAVO deck (Peltier at 4° C.).
   b. the pre-prepared proprietary [protein precipitation-metabolite extraction] formulation “crash” stock was taken from −20° C. storage, stirred, and a PP 96 well plate filled with the appropriate volumes, the “crash” plate is then put on the robot deck.
c) The Bravo protocol is then initiated, the critical steps of this process are:
   a. Draw up 140 μl of SIL-IS from the filled column of the SIL-IS plate and sequentially dispense 10 μl in each of the specimen vials.
   b. Fortified specimens will then be vortexed, on deck, for 5 min at 1200 rpm
   c. Addition of the “crash” solution; this part of the sample preparation is performed in two separate steps
      i. First step: addition of 200 μl “crash” solution, followed by on deck vortexing for 1 minute at 1200 rpm,
      ii. Second step: addition of 140 μl “crash” solution followed by vortexing for 4 minutes at 1000 rpm
d) The specimen plate is then removed from the BRAVO robot and vortexed at 4° C. for 10 min followed by 2 min sonication
e) Transfer of the specimen plate to the freezer, where they are kept at −20° C. for 20 minutes to maximize protein precipitation.
f) After precipitation, the specimen vials are centrifuged at 4° C. for 20 min at a speed of 8000 rpm, then they are returned to the BRAVO robot; the specimen plate is put on the Peltier station at 4° C.
g) Splitting of the supernatant (i.e., the metabolite extract) in two different aliquots to enable the separate analysis of the Hydrophobic and Hydrophilic compounds. Hereto, 240 μl of supernatant is aspirated and 120 μl dispensed is twice, into separate PP 96-well plates (duplicate “specimen extract” plates).
h) The specimen extract plates are then dried by means of vacuum evaporation at 40° C. for 60 minutes. Typically, 1 dried specimen extract plate is transferred to −80° C. until further analysis, the other specimen extract plate is returned to the BRAVO robot for re-constitution, readying the extracted specimens for LC-MS/MS analysis

G. The use of specific Quality Assurance procedures to avoid the introduction of experimental bias and to assure the quality of the quantification of the metabolites of interest.

These procedures define for instance: Analytical Batch Size and batch composition, Number and type of Quality Controls Samples, Criteria for acceptance of data read-outs, Operator blinding, designing sufficiently powered studies, selection of the appropriate study samples. To avoid experimental bias, specific methods are used to randomize the study samples. The lack of bias in sample order is then confirmed using the appropriate statistical tests.

Upon signal processing of the mass spectrometric data, specific post-analysis Quality Assurance methods are applied to assess per metabolite of interest: the data missing-ness rate across a clinical study, the presence of any (unwarranted) experimental bias, eventual signal drift, and the appropriateness of the chosen quantitative read-out (i.e., “metabolite quantifier ion/selected SIL-IS quantifier ion ratio”). Where necessary, alternative quantitative read-outs can be selected. Review of the analyte quantitation is routinely performed to quantify the stability and robustness. In the event there are some inter-day batch drift observed, an appropriate correction can be applied. The appropriate quantification metric is established for each metabolite of interest. Following quality control and the selection of the most robust quantification metric, the imprecision of each metabolite quantification will be gauged, by calculating coefficients of Variance (% CV), using the available QC samples and/or replicate measurements.

H. The application of a set of selection criteria (“Quality Stage-Gate criteria”) is used to determine which metabolites of interest can be progressed to biomarker performance analysis.

Typically, but non-limiting, precision, specificity and missingness criteria are considered. Alternatively, imputation of missing values can also be considered 57. Examples of typical precision limits are e.g., % CV<=15%, or <=20% CV or <=25%. The appropriate Quality Stage-Gate criteria are specifically established for each study of biospecimens and can vary per metabolite of interest. This step will define which metabolites of interest can be progressed to the next steps and be used in multi-variable prognostic/diagnostic test discovery; and will vary per study of biospecimens.

### Analytical Results:

54 metabolites were assayed in all specimens. Following quality control and the selection for each metabolite of interest the most robust quantification metric, the imprecision of the metabolite quantifications was gauged. Metabolites quantifications were only considered for univariable and multivariable analyses if the data missing rate across the study for a metabolite of interest <=20% AND if % CV<=25%. Table 6 reports the 44 metabolites selected, together with the precision (% CV) of the selected metabolite quantification metric. The % CV was calculated based on the 88 replicate samples analyzed across the study (randomly distributed throughout).

Cotinine quantification is the exception to the missing-ness criterion.

### Cotinine

Cotinine is an alkaloid found in tobacco and is also the predominant metabolite of nicotine. Cotinine has an in vivo half-life of approximately 20 hours and is typically detectable for several days (up to one week) after exposure to tobacco. The level of Cotinine in the blood, saliva, and urine is proportionate to the amount of exposure to tobacco smoke, so it is a valuable indicator of tobacco smoke exposure, including secondary (passive) smoke. Whilst smoking is a risk factor of interest for the prediction of pre-eclampsia, it might be prone to under-reporting. Previous studies by the applicants have shown that the bimodal distribution of the relative concentration of Cotinine correlates with reports smoking status. It was found that a cut off at the relative concentration of 10⁻¹. segregated non-smokers from smokers. Further, previous studies have shown that missing quantitations for cotinine were associated with non-smoker status. These observations were confirmed in the present study. The missing quantitations for cotinine were therefore imputed with the lowest observed measured and the cotinine was binarized using the above-mentioned threshold.

### Statistical Analysis

The prediction of pre-eclampsia within the obese using single variables as well as panels of variables was estimated. The following parameters were studied with the aim to discover specific combinations of variables relevant to the prediction of pre-eclampsia within the obese pregnant population:
Forty-four putative blood-borne metabolite biomarkers (Tables 3&6), assayed by means of the purposely developed analytical pipeline met the quality criteria (<20% missingness, with the exception of Cotinine, AND % CV<=25%), see earlier,
One well-studied blood-borne protein biomarker implicated in pre-eclampsia, i.e., Placental Growth Factor (PIGF, PGF (gene)). This protein biomarker was analysed using the Alere Triage meter Pro Fluorescence assay, as reported in (Vieira et al. 2018), in blood samples collected at the same gestational age as the ones used for metabolite analyses. Herein this variable is coded as PIGF.
A number of known clinical risk factors were also considered (Table 7), these were selected on the basis they would be universally easily accessible to primary care providers and/or could be replaced by a biomarker read-out, e.g., measurement of Cotinine to establish smoking status.

TABLE 7
Known clinical risk factors and routine lab analyses considered
as covariates in multi-variable modelling; lab analyses
are performed using blood specimens collected at same
gestational age as the ones used for metabolite analyses.

| Parameter | Description |
|---|---|
| Mat_age | Maternal age |
| Mat_age_GE35 | Maternal age greater than or equal to 35 years |
| Mat_age_LT25 | Maternal age lower than 25 years |
| Mat_chol | Maternal cholesterol at first visit |
| Ma_hdl | Maternal HDL at first visit |
| Mat_ldl | Maternal LDL at first visit |
| Mat_ldl_hdl | Maternal HDL/HDL at first visit |
| Mat_trig | Maternal triglycerides at first visit |
| Mat_bmi | BMI at first visit |
| Mat_prev_pet | Pre-eclampsia in any previous pregnancy |
| fh_any_pet | Family history of Pre-eclampsia (Mother of |
| | pregnant woman, sister of pregnant woman) |
| Mat_dbp_m (BP) | Maternal diastolic blood pressure at baseline, |
| | calculated from duplicate measurements |
| Mat_sbp_m (BP) | Maternal systolic blood pressure at baseline, |
| | calculated from duplicate measurements |
| gaw_1st_visit | Gestation at first visit in weeks |
| uncomp_pregn_score | Multivariable Risk score to predict an |
| | uncomplicated pregnancy in obese pregnant |
| | women ⁴⁷ |

The potential of these parameters to predict pre-eclampsia was studied in distinct set of samples as mentioned previously:
A random selection of 645 control patients and 65 case patients (Table 1).
A range of nested subpopulations (strata), and sub-outcomes within this cohort, as elaborated in Table 2).

### Confirmation of Non-Bias in the Data Set

Association between the mass spectrometry readouts and clinical variables, routine lab analyses and experimental parameters (e.g., order of clinical samples in the study) was checked. The association between analyte concentration or relative concentration and experimental parameters was tested (Chi-squared, Spearman Correlation, Mann Whitney U and Kruskal-Wallis tests, multiple testing correction Benjamini and Hochberg's method, p<0.05). A significant relationship was found with the date at which the sample was first frozen at the clinical collection centres for the following analytes, Sphingosine 1-phosphate, 1-Palmitoyl-2-hydroxy-sn-glycero-3-phosphocholine, Sphinganine-1-phosphate, and Biliverdin. This indicates that the concentration of these analytes varies as a function of storage duration. This phenomenon has been described elsewhere for a range of analytes. Since duration of the storage is independent from the outcome and other clinical parameters (data not shown), this technical bias was considered as an increased random variability in the quantitation of the affected analytes.

### Selection of Duplicates

As part of the quality control measures of the study, some clinical samples were measured twice. For these samples, only one of the two duplicate measurements was randomly selected for further analysis.

### Methods Applied to Select Predictors and Predictor Combinations

The dependencies of each analyte quantification on common patient characteristics such as clinical center, bmi, ethnicity, age etc or gestational age at sampling are also assessed. The analytes that do show a significant dependency (Spearman Correlation, Mann Whitney U, Benjamini, Hochberg and Yekutieli, p<0.01) on these factors are normalised using a multiple-of-median (MoM) methodology. For the computation of predictive models, both MoM-normalised and non-normalised variables were considered. The (combinations of) predictors as found by the inventors and reported here, will therefore explicitly include both MoM-normalised and non-MoM normalised variables. For clarity of presentation, no distinction is made between MoM-normalised and non-MoM normalised variables in the univariable and multivariable combinations; only the data for non-MoM normalised variables are given. One exception is made for PIGF: both MoM-normalised and non-MoM normalised read-outs were considered in the multivariable analyses; albeit they are considered mutually exclusive predictors, i.e., a model cannot feature both the MoM-normalised and non-MoM normalised read-out for PIGF.

The ability of the variables to predict the outcome (pre-eclampsia in obese patients) was identified using two approaches.

Firstly, the discriminative performance of each individual variable was quantified using the area under the receiver operating curve, (AUROC or AUC) 12. An AUC of 0.5 or lower indicates an absence of predictive power for the outcome. In order to select robust predictors, only the variables that had a lower limit of the 95% confidence interval of AUC greater or equal to 0.50 were selected as predictive markers for pre-eclampsia in obese patients. In other words, variables were considered univariable predictors if their AUC was significantly higher than 0.5 (p<0.05).

Secondly, Partial Least Squares-Discriminant Analysis (PLS-DA) models were generated for all combinations of two to four analytes and possible clinical predictors as identified earlier. For the avoidance of doubt, statistical techniques other than PLS-DA can also be employed, and more than 4 predictors can also be considered. The following statistical methodology was applied.
1. The concentrations and relative concentrations of PIGF, the routine lab analyses and the metabolites were log-transformed before modelling.
2. PLS-DA multivariable models were made by selecting variables as follows:
   1. 1 to 4 variables.
   2. All possible combinations of variables.
   3. Shall constitute at most one clinical risk factor or routine lab analyte, therefore variables such as Mat_chol, Mat_hdl, Mat_Idl, Mat_Idl_hdl or Mat_trig are not combined with a clinical risk factor.
3. A three-fold cross validation was performed stratifying over the outcome (pre-eclampsia).
4. Models were additionally trained on the entire dataset.
5. For all models, the p values of the Mann Whitney U tests per patient stratum and outcome were adjusted for multiple testing using Benjamini & Hochberg step-up False Discovery Rate-controlling procedure.
6. For all models the likelihood ratio of the raw and corrected p values were computed. The likelihood ratio is the ratio between the statistic value for the given model and the highest value amongst the parent models. Parent models are all models with fewer predictors than the given model and whose predictors are all used in the given model. The applicants called this likelihood ratio: “improvement”.
   Note: Statistical metrics as obtained from the full data set (i.e., train performance) are identifiable by the interjection “all” in their name; Statistical metrics as derived from the three-fold cross validation data (i.e., cross-validation mean test performance) by the interjection “mean” in their name. The suffix “mtc” indicates that the statistic is corrected for multiple testing. p Values were combined using Fisher's method.
7. Models were selected for reporting based on any of the below criteria:
   1. Models with one predictor (single markers; cf. first approach)
   2. Multivariable Models with 2 to 4 predictors wherefore: (predict.mean.pValue.mtc <=0.05)
      AND (predict.all.pValue.mtc <=0.05)
      AND (predict.mean.pValue.mtc_improvement <=0.10)
      AND (predict.all.pValue.mtc_improvement <=0.10)
   3. Reference models, i.e. models solely based on blood pressure values and/or PIGF These models selected respectively correspond to:
   1. Single predictors for PE in the obese.
   2. Sparse models that have a statistically significant prognostic performance for the outcome within the stratum studied.
   3. Models used as reference to estimate the relevance of models selected by the other criteria.

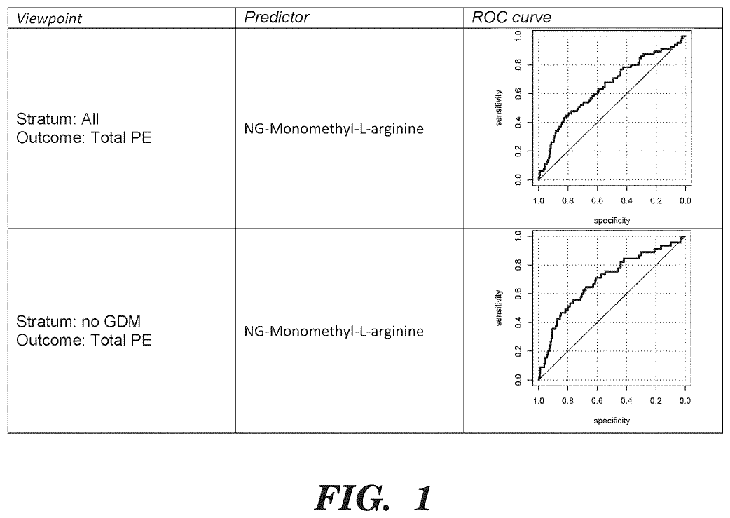

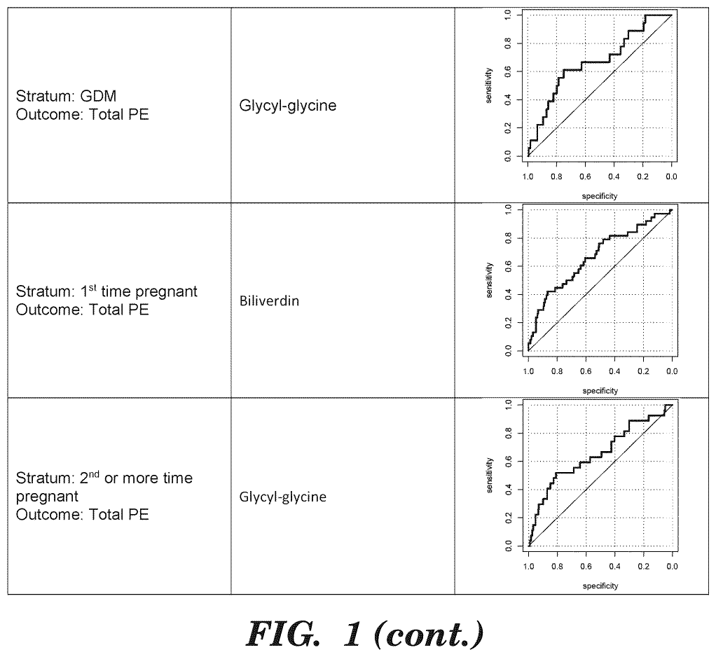

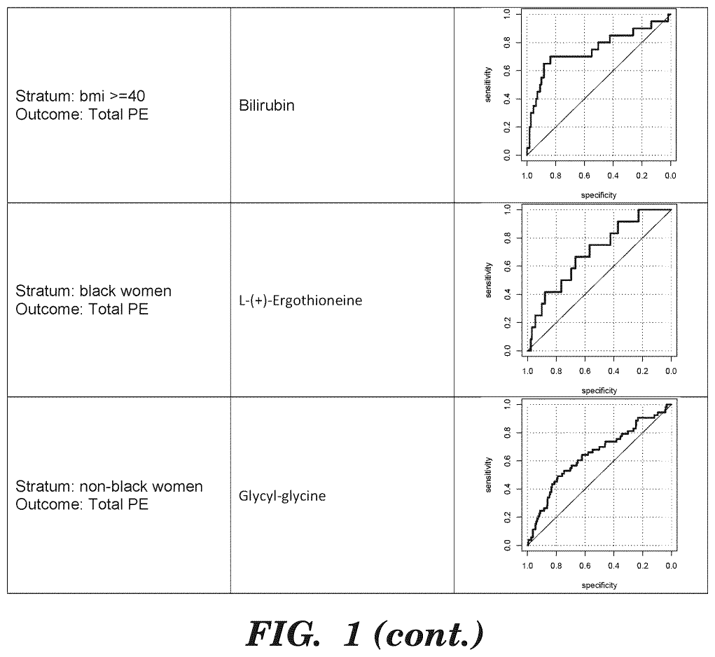

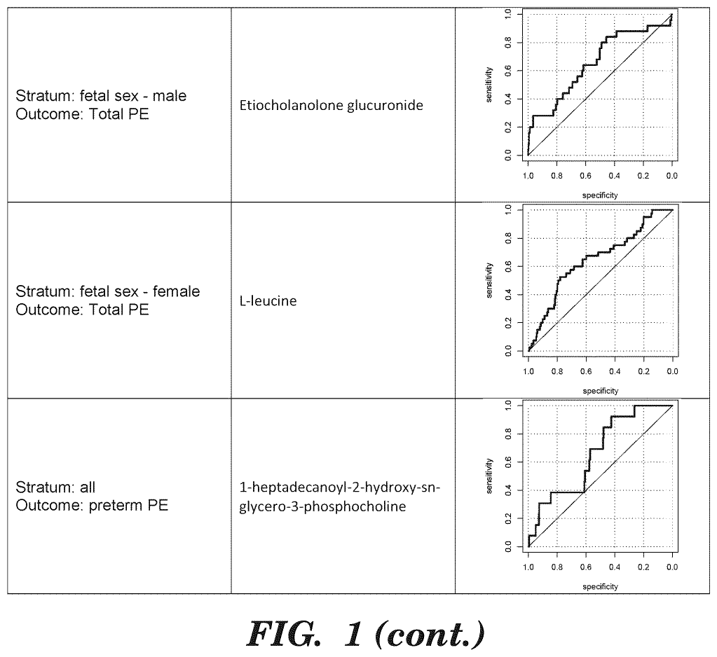

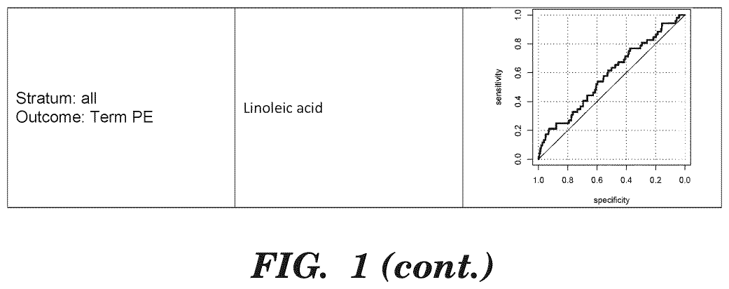

Univariable predictors are tabulated in Example 2. Models as per point 2 wherefore: (predict.mean.AUC >=0.75) AND (predict.all.AUC >=0.75) are tabulated in Example 3. Receiver operating characteristic (ROC) curves for exemplary single metabolite predictors (point 1) and exemplary multivariable predictors (point 2) are also presented in graphic format in the respective FIGS. 1 & 2. These are indicated by an asterisk (*) in the tables of Example 2 and Example 3.
8. The applicants then ranked the predictors based on the rate (frequency) of occurrence in models selected with criterium 2 per stratum and outcome.

### Example 2—Results Univariable Analyses

### Single Predictor Selection

Given the applicants idea that the prediction of pre-eclampsia in the obese pregnant may require for different predictors, and combinations thereof, depending on specific patient populations and/or disease sub-types, this was considered in the univariable analyses. Firstly, the applicants investigated variables, and metabolite variables more specifically, that can have merits in predicting pre-eclampsia in all obese pregnant women. Then, the applicants aimed to identify predictors specific to any of the substrata defined earlier. To identify predictors which are truly sub-group specific, and which may have been missed by treating the obese pregnancy population as a homogenous group, their prognostic performance is compared to their performance in predicting PE in the complete obese population (stratum: all). If they do not have significant prognostic performance in the complete obese population, they are considered sub-group specific. The same rationale is applied for predictors as found for term- and preterm PE vs prediction of total pre-eclampsia in all. Any rankings in the below tables are based on the lower limit of the 95% CI (ICI) of the AUC as found for the prediction of PE in all pregnant obese (stratum: all). Non-significant predictors, based on the lower limit of the 95% CI, are shown in italicized bold text. For information purposes, the upper limit of the 95% CI (uCl) of the AUC is also presented. Information on whether the median levels of a variable are higher (UP) or lower (DOWN) in the future PE cases compared to the future controls are also given for these variables with significant prognostic performance.

Exemplary ROC curves for univariable predictors across all viewpoints are presented in FIG. 1; the univariable predictors selected for presented in FIG. 1 are indicated in the tables of Example 2 by an Asterix (*)

In addition to some known clinical variables and routine lab analyses, 15 metabolites with univariable prognostic performance to prognose PE in obese pregnant women were found significant (p<0.05), of which 7 have an AUC >=0.60 (Table 8).

In addition to some known clinical variables (blood pressures, Mat_prev_pet) and routine lab analyses, 12 metabolites with significant univariable prognostic performance to prognose PE in obese pregnant women who do not develop GDM as a comorbidity were found (p<0.05). All but one predictor, were also found for the prediction of PE in all; Stearic acid, is only significant in the stratum no-GDM (Table 9).

Differently, only blood pressures and 3 metabolites with significant univariable prognostic performance to prognose PE in obese pregnant women who develop GDM as a comorbidity, were found (p<0.05); Asymmetric dimethylarginine and Taurine were only found significant to the prediction of PE in in obese pregnant women who develop GDM (Table 9).

Based on the data in Table 9, NG-Monomethyl-L-arginine, Dilinoleoyl-glycerol, Mat_trig, PIGF, Octadecenoic acid, Hexadecanoic acid, Linoleic acid and Stearic acid appear mostly specific to predicting PE in women that will not develop GDM as comorbidity (p<0.05). Differently, Asymmetric dimethylarginine and Taurine were only shown to predict PE in these women that will develop GDM as a comorbidity during their pregnancies. Also, there is a clear difference within the blood pressure measurements; whereas the diastolic blood pressure is the best predictor for PE in all obese pregnancies, and these obese pregnant women, who will not develop GDM as a comorbidity, the systolic blood pressure appears the best single predictor for PE in these women that go on to develop both PE and GDM.

In addition to some known clinical variables and routine lab analyses, 8 metabolites with significant univariable prognostic performance to prognose PE in the 1st time pregnant obese women were discovered. All but one predictor, were also found for the prediction of PE in all; L-alanine, is only significant in the stratum nulliparous (Table 10).

In addition to some known clinical variables and routine lab analyses, 7 metabolites with univariable prognostic performance to prognose PE in the 2nd time or more pregnant obese women. All but one predictor, were also found for the prediction of PE in all; 8,11,14 Eicosatrienoic acid, is only significant in the stratum multiparous (Table 11).

Based on the data, NG-Monomethyl-L-arginine and Biliverdin appear mostly specific to predicting PE in the 1st time pregnant obese women; while, 8,11,14 Eicosatrienoic appears more specific to predict PE in multiparous pregnant obese women.

Also, there is a clear difference within the blood pressure measurements. Blood pressures appear to have strong predictive performance for predicting PE in the 2nd time or more pregnant obese women, but far less so in the 1st time pregnant obese women. It is also clear, that being exposed to pre-eclampsia in a previous pregnancy is a fair predictor in the multiparous. Together with the observation that blood pressures are not so predictive, this fortifies the concept that prediction of PE in 1st time pregnant women requires for better biomarkers as clinical risk factors are less relevant in this group.

In addition to blood pressures, 6 metabolites with significant univariable prognostic performance to prognose PE in the morbidly obese pregnant women were identified. Four of these, were also found for the prediction of PE in all; Taurine and L-arginine are only significant in the stratum bmi>40 (Table 11).

Based on the data, Bilirubin, Biliverdin, Taurine and L-arginine are more specific to predicting PE in the morbidly obese women, as a distinct subgroup in the obese pregnant. Strikingly, NG-Monomethyl-L-arginine, does not appear to be a predictor of PE for morbidly obese women, it's predictive performance for PE appears restricted to the non-morbidly obese pregnant women.

The predictors for the non-black obese pregnant women are largely the same as these identified for the prediction of PE in all obese pregnant women.

Two metabolites have a remarkably high performance for the prediction of PE in the black obese women, namely L-(+)-Ergothioneine and Stearoylcarnitine; the latter not reaching significance when considered in all. In addition, the routine lab analyses Mat_hdl and Mat_trig are also far more predictive in the black obese women compare to the non-black obese women (Table 12).

Inversely, the metabolites Met_XXX, Glycyl-glycine, Linoleic acid and interestingly the blood pressure measurement Mat_sbp_m appear particularly ineffective to predict PE in the black obese women, with their predictive merits for PE restricted to non-black obese women (Table 12).

No specific predictors are found, compared to prediction of PE in all the pregnant obese (stratum: all) when taking fetal sex into account (Table 13).

Yet, some predictors appear more specific to predicting PE depending on the fetal sex of the offspring. The metabolites L-leucine and L-Isoleucine and the routine clinical lab analyte Mat_hdl appear more specific to predicting PE in obese women pregnant with female fetuses. In this population PIGF appears also more predictive for PE in obese pregnant women carrying a female fetus.

Interestingly, the blood pressure measurements Mat_dbp_m and Mat_sbp_m, as well as the routine clinical lab analyte Mat_trig appear far more specific to predicting PE in obese women pregnant with male fetuses.

Compared to prediction of PE in all the pregnant obese (stratum: all), only 1 additional biomarker was found when differentiating the sub-outcomes Preterm Pre-eclampsia and Term Pre-eclampsia, i.e. Urea which only reaches significance for predicting term PE in the obese pregnant women (Table 14).

Yet, some predictors appear more specific to predicting PE depending the sub-type of PE. In agreement with literature PIGF is more specific for PE manifesting earlier in pregnancy. Another predictor identified to be more specific to Preterm PE than to Term PE is 1-heptadecanoyl-2-hydroxy-sn-glycero-3-phosphocholine. In the studied pregnancy population, preterm PE was also associated with a history of pre-eclampsia in a previous pregnancy. On the other hand, Linoleic acid, bilirubin and Mat_bmi appear to be more specific to the prediction of term PE in the obese.

Conversely, Mat_bmi may be a better predictor for term PE than preterm PE in the obese pregnant population.

Summary Single Predictors for PE in the Obese: Metabolites with Stand-Alone Predictive/Prognostic Performance.

In addition to the metabolite predictors NG-Monomethyl-L-arginine, Biliverdin, Glycyl-glycine, Dilinoleoyl-glycerol, Etiocholanolone glucuronide, Bilirubin, L-leucine, 25-Hydroxyvitamin D3, 1-heptadecanoyl-2-hydroxy-sn-glycero-3-phosphocholine, L-(+)-Ergothioneine, Octadecenoic acid, Met_XXX, Hexadecanoic acid, Linoleic acid, L-Isoleucine which have stand-alone predictive performance predict PE in the obese pregnancy population. An additional set of metabolite predictors relevant to predict PE in the obese where discovered by looking into specific sub-populations within the obese pregnant and/or specific sub-types of PE. These include Stearic acid, Asymmetric dimethylarginine, Taurine, 8,11,14 Eicosatrienoic acid, L-alanine, Taurine, L-arginine, Stearoylcarnitine and Urea.

Within these, the following metabolite predictors have strong prognostic performances (AUROC>=0.65) to predict PE in the obese population as assessed in the various viewpoints taken by the applicants: NG-Monomethyl-L-arginine, Glycyl-glycine, Dilinoleoyl-glycerol, Biliverdin, Asymmetric dimethylarginine, Taurine, Bilirubin, Etiocholanolone glucuronide, L-leucine, L-(+)-Ergothioneine, and 1-heptadecanoyl-2-hydroxy-sn-glycero-3-phosphocholine.

Within these, the following metabolite predictors have particularly strong prognostic performances (AUROC>=0.70) to predict PE in the obese population as assessed in the various viewpoints taken by the applicants: NG-Monomethyl-L-arginine, Biliverdin, Bilirubin, and L-(+)-Ergothioneine.

### Example 3—Results Multivariable Analyses

The concept of creating a model space containing all possible prediction models with 2 to 4 predictors followed by the exploring this model space using strict statistical criteria as explained earlier, enables the discovery of robust synergistic predictor potential, i.e., these combinations of predictors that significantly improve the prognostic performance of any of the constituting parent models. For instance, a prognostic model ABC constituting the predictors A, B and C is considered an improved model only if the prognostic performance of ABC is significantly better than either of the model AB, BC and AC, based on the criteria explained earlier.

Again, the applicants applied a variety of viewpoints in addition to the prediction of PE in all obese women. The applicants did this to ensure that the ensuing selection of predictors, and the combinations of predictors within this selection, will predict PE in the pregnant obese irrespective of the patient subgroup (phenotypes, strata) a woman is part of, or independent of the PE sub-diseases she is at risk of developing.

Across all viewpoints considered, 1224 models of 2 to 4 predictors were found which complied with the strict statistical criteria applied. It will be obvious to someone skilled in the art, that it will be harder to meet the criteria when the number of participants (cases and controls) in a stratum is smaller, as such the methodology applied will protect against overfitting of the data. At the same time, lack of models in a stratum and/or outcome cannot be interpreted that no synergistic models do exist, only that models are not selected due to a lack of statistical power in the current sample set and the stringent selection criteria applied.

By ranking single predictors based on the rate of occurrence in models selected per stratum and outcome, the applicants identified these predictors that have additive potential to predicting PE, per stratum and/or per outcome. It will be obvious to someone skilled in the art, that further improving by adding one predictor upon particularly strong single predictors and/or particularly strong 2 predictor models, and/or particularly strong 3 predictor models, will be difficult within the set of stringent statistical criteria applied. Therefore, the fact that specific predictors are less frequent, e.g. part of only less than 30% of selected models, or less than 20% of models, or less than 10% of models, or less than 5% of models per stratum or outcome, does not preclude these predictors as being relevant to predicting PE in the obese pregnant women.

In Table 15 (Annex), the frequency of all predictors across all viewpoints, as well as per viewpoint (stratum and outcome) is given. The different viewpoint analyses are represented using the follow column header letter codes.

| Letter | Population sub-group/pre-eclampsia | Cases | Controls |
|---|---|---|---|
| code | sub-outcome (italic) | (N=) | (N=) |
| A | Prognosis of PE in all the obese pregnant | 65 | 645 |
| B | Prognosis of PE in the obese pregnant who | 45 | 465 |
| | don't develop the GDM co-morbidity | | |
| C | Prognosis of PE in the obese pregnant who do | 18 | 169 |
| | develop the GDM co-morbidity | | |
| D | Prognosis of PE in the first-time pregnant | 38 | 287 |
| | women | | |
| E | Prognosis of PE in the non-first-time pregnant | 27 | 358 |
| | women, i.e., within women who have been | | |
| | pregnant before | | |
| F | Prognosis of PE in the morbidly obese pregnant | 20 | 111 |
| | women, i.e., women with a bmi >= 40 | | |
| G | Prognosis of PE in pregnant women of black | 12 | 132 |
| | ethnicity | | |
| H | Prognosis of PE in pregnant women of non- | 53 | 513 |
| | black ethnicity | | |
| I | Prognosis of PE in pregnant women with male | 25 | 328 |
| | offspring | | |
| J | Prognosis of PE in pregnant women with | 40 | 315 |
| | female offspring | | |
| K | Prognosis of preterm PE. i.e., PE warranting a | 13 | 645 |
| | delivery before 37 weeks of gestation | | |
| L | Prognosis of term PE. i.e., PE not leading to a | 52 | 645 |
| | delivery before 37 weeks of gestation | | |

From Table 15, it is clear that notably the metabolites, Eicosapentaenoic acid, Stearic acid, 1-Palmitoyl-2-hydroxy-sn-glycero-3-phosphocholine, Myristic acid, L-Palmitoylcarnitine, Arachidonic acid, Choline, Docosahexaenoic acid, L-methionine, 3-Hydroxybutanoic acid, and to a lesser extent the metabolites, 2-Hydroxybutanoic acid, Decanoylcarnitine, L-Acetylcarnitine, Citrulline, Dodecanoyl-I-carnitine and Sphingosine 1-phosphate have a synergistic prognostic utility in multivariable models, despite the fact they did not exhibit univariable prognostic performance.

Depending on the demographic constitution of the obese pregnancy population, accurate prediction of pre-eclampsia risk will be possible by means of multivariable models that contain at least two or more, or at least three or more, or at least four or more, or at least 5 or more, etc of the predictors as tabulated in table 15, whereby at least one or more of, or at least two or more, or at least three or more or at least four or more, etc is a metabolite of the group Biliverdin, Met_XXX, Glycyl-glycine, NG-Monomethyl-L-arginine, Etiocholanolone glucuronide, Eicosapentaenoic acid, Dilinoleoyl-glycerol, L-leucine, Hexadecanoic acid, 25-Hydroxyvitamin D3, Linoleic acid, Octadecenoic acid, Stearoylcarnitine, Stearic acid, 8,11,14 Eicosatrienoic acid, 1-Palmitoyl-2-hydroxy-sn-glycero-3-phosphocholine, L-Isoleucine, Bilirubin, L-arginine, L-(+)-Ergothioneine, Myristic acid, L-Palmitoylcarnitine, Arachidonic acid, Urea, Choline, Taurine, Docosahexaenoic acid, Asymmetric dimethylarginine, L-methionine, 3-Hydroxybutanoic acid, 2-Hydroxybutanoic acid, 1-heptadecanoyl-2-hydroxy-sn-glycero-3-phosphocholine, Decanoylcarnitine, L-Acetylcarnitine, Citrulline, Dodecanoyl-I-carnitine, Sphingosine 1-phosphate, L-alanine.

For illustrative purposes only, the applicants present in the Tables 16 to 26 (annex) here exemplary combinations of 2 to 4 predictors among the 1224 multivariable models that met the stringent performance and improvement criteria as elaborated earlier, which result in an AUROC >=0.75 for both the full data set (all.AUC), as well in the 3 fold cross validation (mean.AUC) for the different viewpoints considered: i.e., Prognosis of PE in all the obese pregnant; Prognosis of PE in the obese pregnant who don't develop the GDM co-morbidity

Prognosis of PE in the obese pregnant who do develop the GDM co-morbidity; Prognosis of PE in the first-time pregnant women; Prognosis of PE in the non-first-time pregnant women, i.e., within women who have been pregnant before; Prognosis of PE in the morbidly obese pregnant women, i.e., women with a bmi>=40; Prognosis of PE in pregnant women of black ethnicity; Prognosis of PE in pregnant women of non-black ethnicity

Prognosis of PE in pregnant women with male offspring; Prognosis of PE in pregnant women with female offspring; Prognosis of term PE. i.e., PE not leading to a delivery before 37 weeks of gestation

Exemplary ROC curves for multivariable predictors across all viewpoints assessed are presented in FIG. 2; the multivariable predictors selected for presented in FIG. 2 are indicated in the tables of Example 3 by an Asterix (*).

Applying the stringent criteria as outlined earlier, no multivariable models were found for the prediction of Preterm PE in the obese pregnant women. This does not preclude that specific combinations of predictors as listed in Table 15 cannot prognose preterm PE in the obese pregnant women.

### Example 4—Results Multivariable Analyses—Delineation of Rule-In and Rule-Out Tests

The AUROC (AUC), essentially a measure of discrimination, corresponds to the probability that a classifier will correctly rank a randomly chosen person with the condition higher than a randomly chosen person without the condition.⁶⁴ The AUROC may not be optimal in assessing prognostic models or models that stratify individuals into risk categories. Clinical decisions and access to certain clinical care pathways are mostly governed by weighing the benefits versus the costs at the level of the intended-use population. For a so-called “rule-in” test, the benefit of the early detection of risk in those who will develop the disease (true positives) needs to be balanced against the cost of wrongly identifying individuals as being at high risk (false positives). Vice versa, for a “rule-out” test, the benefits of finding true negatives will be weighed against wrongly identifying false negatives as being at low risk. Rule-in tests and Rule-out test are characterised by skewed ROC curves as illustrated in FIGS. 1 and 5 in the applicants scientific paper.

Therefore, in a separate exercise the applicants looked whether the selection of predictors considered would also enable the development of particular Rule-in or Rule-out models. To do so, the applicants applied a similar statistical methodology as outlined in Example 1 (statistical analysis-Methods applied to select predictors and predictor combinations), yet a different set of threshold criteria was applied to find within the comprehensive multi-variable model space either Rule-in or Rule-out models. In brief, the statistical methodology applies was the following:

Again the concentrations and relative concentrations of PIGF, the routine lab analyses and the metabolites were log-transformed before modelling; PLS-DA models were generated for all combinations of two to four variables once more. A three-fold cross validation was performed using a block-randomization over the outcome (pre-eclampsia).

The predictive performance for the outcome of each predictor panel was quantified using the mean using Fisher's method over the cross-validation of the p value of the Mann Whitney test comparing the model score between case and control patients corrected for multiple testing using Benjamini and Hochberg method. For each models a likelihood ratio here named the “improvement” was computed. For a marker panel, the improvement is the ratio between the mentioned corrected p value of the panel and the lowest of the p values of panels that are constituted of a subset of its predictors. This statistic indicates whether a panel improves on the performance of its components.

Further the discriminative performance of panels was quantified using the mean over the cross validation of both sensitivity at 80% specificity and specificity at 80% sensitivity with their respective 90% confidence interval. These two statistics gives an estimate of the clinical usefulness of panels in as a “rule-in” and or or “rule-out” classifier⁶⁶.

Marker panels were selected if
1) the mean p value was lower than 0.05 and
2) the improvement was lower than 0.50 and
3) the rule-in or rule-out performance at 80% sens or 80% spec is greater than 0.50 and
4) the lower confidence interval for the rule-in or rule-out performance is greater than 0.35.

These criteria ensure that using the panel a score can be derived that is different for case and control patients, that the panel is sparse and that the panel has some clinical usefulness.

The Applicants found that the predictors as identified in Table 15 also enable the development of robust prognostic models with particular rule-in and/or rule-out merits. Combinations of predictors for all viewpoints were found, we illustrate in the tables 27 to 32 (annex) the potential for specific rule-in and rule-out multivariable models for a number of viewpoints. ROC curves for exemplary multivariable Rule-in and Rule-out predictors are also presented in graphic format in FIG. 3. In the tables, Rule-in predictors selected for graphical presentation are indicated by an asterisk (*), Rule-out predictors are indicated by a double asterisk (**).

Depending on the demographic constitution of the obese pregnancy population, accurate rule-in prediction and/or rule-out of pre-eclampsia risk will be possible by means of multivariable models that contain at least two or more, or at least three or more, or at least four or more, or at least 5 or more, etc of the predictors as tabulated in table 15, whereby at least one or more of, or at least two or more, or at least three or more or at least four or more, etc is a metabolite of the group Biliverdin, Met_XXX, Glycyl-glycine, NG-Monomethyl-L-arginine, Etiocholanolone glucuronide, Eicosapentaenoic acid, Dilinoleoyl-glycerol, L-leucine, Hexadecanoic acid, 25-Hydroxyvitamin D3, Linoleic acid, Octadecenoic acid, Stearoylcarnitine, Stearic acid, 8, 11, 14 Eicosatrienoic acid, 1-Palmitoyl-2-hydroxy-sn-glycero-3-phosphocholine, L-Isoleucine, Bilirubin, L-arginine, L-(+)-Ergothioneine, Myristic acid, L-Palmitoylcarnitine, Arachidonic acid, Urea, Choline, Taurine, Docosahexaenoic acid, Asymmetric dimethylarginine, L-methionine, 3-Hydroxybutanoic acid, 2-Hydroxybutanoic acid, 1-heptadecanoyl-2-hydroxy-sn-glycero-3-phosphocholine, Decanoylcarnitine, L-Acetylcarnitine, Citrulline, Dodecanoyl-I-carnitine, Sphingosine 1-phosphate, L-alanine.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

### Annex

TABLE 3
Metabolites considered:

| Metabolite | Metabolite Class | HMDB | CAS | MW |
|---|---|---|---|---|
| 25-Hydroxyvitamin D₃ | Vitamin D and derivatives | 0003550 | CAS 63283-36-3 | 400.6371 |
| 2-Hydroxybutanoic acid | Keto- or Hydroxy fatty acids | 0000008 | CAS 5094-24-6 | 104.1045 |
| 3-Hydroxybutanoic acid | Keto- or Hydroxy fatty acids | 0000357 | CAS 300-85-6 | 104.1045 |
| L-alanine | Amino acids | 0000161 | CAS 56-41-7 | 89.0932 |
| Arachidonic acid | (Unsaturated) Fatty acids | 0001043 | CAS 506-32-1 | 304.4669 |
| L-arginine | Amino acids | 0000517 | CAS 74-79-3 | 174.201 |
| L-leucine | Amino acids | 0000687 | CAS 61-90-5 | 131.1729 |
| 8,11,14 Eicosatrienoic acid | (Unsaturated) Fatty acids | 0002925 | CAS 1783-84-2 | 306.4828 |
| Citrulline | Amino acids | 0000904 | CAS 372-75-8 | 175.1857 |
| Decanoylcarnitine | Carnitines | 0000651 | CAS 1492-27-9 | 315.4482 |
| Dodecanoyl-l-carnitine | Carnitines | 0002250 | CAS 25518-54-1 | 343.5014 |
| Docosahexaenoic acid | (Unsaturated) Fatty acids | 0002183 | CAS 6217-54-5 | 328.4883 |
| Dilinoleoyl-glycerol: | Diacylglycerols | 0007248 | CAS 15818-46-9 | 616.9542 |
| 1,3Dilinoleoyl-glycerol and | | | CAS 30606-27-0 | |
| 1,2-Dilinoleoyl-glycerol | | | | |
| (isomer mixture) | | | | |
| Choline | Cholines | 0000097 | CAS 62-49-7 | 104.1708 |
| Glycyl-glycine | Dipeptides | 0011733 | CAS 556-50-3 | 132.1179 |
| Homo-L-arginine | Amino acids | 0000670 | CAS 156-86-5 | 138.2275 |
| Hexadecanoic acid (palmitic acid) | Fatty acids | 0000220 | CAS 57-10-3 | 256.4241 |
| L-Isoleucine | Amino acids | 0000172 | CAS 73-32-5 | 131.1729 |
| Linoleic acid | (Unsaturated) Fatty acids | 0000673 | CAS 60-33-3 | 280.4455 |
| L-methionine | Amino acids | 0000696 | CAS 63-68-3 | 149.211 |
| NG-Monomethyl-L-arginine | Amino acids | 0029416 | CAS 17035-90-4 | 188.2275 |
| Octadecenoic acid (the total of the | (Unsaturated) Fatty acids | 0000207 | CAS 112-80-1 | 282.4614 |
| metabolites: Oleic acid (primary | | | CAS 112-79-8 | |
| contributor (Z)-octadec-9-enoic | | | CAS 506-17-2 | |
| acid), and possibly (E)-octadec- | | | CAS-693-72-1 | |
| 9-enoic acid, (Z,E)-octadec-11- | | | CAS 593-39-5 | |
| enoic acid, (Z,E)-octadec-6-enoic | | | na | |
| acid) | | | | |
| L-Palmitoylcarnitine | Acyl carnitines | 0000222 | CAS 6865-14-1 | 399.6077 |
| Asymmetric dimethylarginine | Amino acids | 0001539 | CAS 30315-93-6 | 202.2541 |
| Sphingosine 1-phosphate | Phosphosphingolipids | 0000277 | CAS 26993-30-6 | 379.4718 |
| Sphinganine-1-phosphate (C18 | Phosphosphingolipids | 0001383 | CAS 19794-97-9 | 381.4877 |
| base) | | | | |
| Symmetric dimethylarginine | Amino acids | 0003334 | CAS 30344-00-4 | 202.2541 |
| Taurine | Amino acids | 0000251 | CAS 107-35-7 | 125.147 |
| Urea | Amino ketones | 0000294 | CAS 57-13-6 | 60.0553 |
| Stearoylcarnitine | Acyl carnitines | 0000848 | CAS 1976-27-8 | 427.6609 |
| Eicosapentaenoic acid | (Unsaturated) Fatty acids | 0001999 | CAS 10417-94-4 | 302.451 |
| 1-heptadecanoyl-2-hydroxy-sn- | Glycerophospholipids | 0012108 | CAS 50930-23-9 | 509.6566 |
| glycero-3-phosphocholine | | | | |
| Bilirubin | (Tetrapyrroles and derivatives) | 0000054 | CAS 635-65-4 | 564.6621 |
| | Bilirubins | | | |
| Biliverdin | (Tetrapyrroles and derivatives) | 0001008 | CAS 114-25-0 | 582.6463 |
| | Bilirubins | | | |
| Etiocholanolone glucuronide | (androgens/steroids) | 0004484 | CAS 3602-09-3 | 466.5644 |
| | Testosterones | | | |
| Cotinine | Pyridines | 0001046 | CAS 486-56-6 | |
| Myristic acid | (Saturated) Fatty acids | 0000806 | CAS 544-63-8 | 228 3709 |
| Stearic acid | (Saturated) Fatty acids | 0000827 | CAS 57-11-4 | 284.4772 |
| L-(+)-Ergothioneine | Amino acids | 0003045 | CAS 497-30-3 | 229.299 |
| Met_XXX | UNKNOWN | | | 366 |
| 1-Palmitoyl-2-hydroxy-sn-glycero- | Glycerophospholipids | 0010382 | CAS 17364-16-8 | 495.6301 |
| 3-phosphocholine (LysoPC(16:0)) | | | | |
| L-Acetylcarnitine | Carnitines | 0000201 | CAS 3040-38-8 | 203.2356 |
| L-Lysine | Amino acids | 0000182 | CAS 56-87-1 | 146.1876 |
| L-Glutamine | Amino acids | 0000641 | CAS 56-85-9 | 146.1445 |

TABLE 4
LC-MRM parameters for the hydrophobic metabolites of interest and associated SIL-IS

| | Rt | Quant/ | MS1 m/z | MS2 m/z | Quant/ | Dwell | Frag | CE | CAV | |
|---|---|---|---|---|---|---|---|---|---|---|
| Metabolite | (min) | Qual | (Res) | (Res) | Qual ratio | (ms) | (V) | (V) | (V) | Polarity |
| 25-Hydroxyvitamin D₃ | 6.6 | Quant | 401.3 (Wide) | 383.3 (Wide) | 23.6 | 20 | 104 | 4 | 2 | Positive |
| | | Qual | 401.3 (Wide) | 365.3 (Wide) | | 20 | 104 | 4 | 2 | Positive |
| Arachidonic acid | 6.8 | Quant | 303.1 (Unit) | 259.1 (Unit) | 34.7 | 3 | 135 | 3 | 2 | Negative |
| | | Qual | 303.1 (Unit) | 59.1 (Unit) | | 3 | 135 | 15 | 2 | Negative |
| 8,11,14 Eicosatrienoic acid | 7.0 | Quant | 305.1 (Unit) | 305.0 (Unit) | 152.7 | 3 | 80 | 1 | 2 | Negative |
| | | Qual | 305.1 (Unit) | 304.9 (Unit) | | 3 | 80 | 0 | 2 | Negative |
| Decanoylcarnitine | 4.7 | Quant | 316.1 (Unit) | 60.1 (Unit) | 59.9 | 3 | 190 | 24 | 2 | Positive |
| | | Qual | 316.1 (Unit) | 257.1 (Unit) | | 3 | 190 | 12 | 2 | Positive |
| Dodecanoyl-l-carnitine (C12) | 5.4 | Quant | 344.1 (Unit) | 85.1 (Unit) | 37.8 | 3 | 140 | 21 | 3 | Positive |
| | | Qual | 344.1 (Unit) | 85.0 (Unit) | | 3 | 140 | 51 | 3 | Positive |
| Docosahexaenoic acid | 6.7 | Quant | 327.1 (Unit) | 283.1 (Unit) | 11.8 | 3 | 80 | 5 | 2 | Negative |
| | | Qual | 327.1 (Unit) | 229.1 (Unit) | | 3 | 80 | 5 | 2 | Negative |
| Dilinoleoyl-glycerol⁺ | 8.8 | Quant | 634.4 (Unit) | 337.5 (Unit) | 185.4 | 3 | 84 | 28 | 2 | Positive |
| | | Qual | 634.4 (Unit) | 599.2 (Unit) | | 3 | 84 | 16 | 2 | Positive |
| Hexadecanoic acid | 7.0 | Quant | 255.1 (Unit) | 255.1 (Unit) | 21.5 | 3 | 130 | 15 | 3 | Negative |
| | | Qual | 255.1 (Unit) | 255.0 (Unit) | | 3 | 130 | 20 | 3 | Negative |
| Linoleic acid | 6.9 | Quant | 279.1 (Unit) | 279.1 (Unit) | 13.8 | 3 | 104 | 10 | 2 | Negative |
| | | Qual | 279.1 (Unit) | 279.0 (Unit) | | 3 | 104 | 20 | 2 | Negative |
| Octadecenoic acid** | 7.1 | Quant | 281.1 (Unit) | 281.1 (Unit) | 22.3 | 3 | 128 | 10 | 3 | Negative |
| | | Qual | 281.1 (Unit) | 281.0 (Unit) | | 3 | 128 | 20 | 3 | Negative |
| L-Palmitoylcarnitine | 6.3 | Quant | 400.2 (Unit) | 60.2 (Unit) | 34.8 | 3 | 110 | 26 | 2 | Positive |
| | | Qual | 400.2 (Unit) | 341.2 (Unit) | | 3 | 110 | 17 | 2 | Positive |
| Sphingosine-1-phosphate | 6.1 | Quant | 380.1 (Unit) | 264.2 (Unit) | 5.9 | 3 | 100 | 11 | 3 | Positive |
| | | Qual | 380.1 (Unit) | 362.2 (Unit) | | 3 | 100 | 11 | 3 | Positive |
| Sphinganine-1-phosphate (C18 base) | 6.2 | Quant | 382.0 (Unit) | 284.0 (Unit) | 43.3 | 3 | 100 | 8 | 3 | Positive |
| | | Qual | 382.0 (Unit) | 266.0 (Unit) | | 3 | 100 | 12 | 3 | Positive |
| Stearoylcarnitine | 6.7 | Quant | 428.3 (Unit) | 85.0 (Unit) | 2.7 | 3 | 130 | 25 | 5 | Positive |
| | | Qual | 428.3 (Unit) | 369.3 (Unit) | | 3 | 130 | 15 | 5 | Positive |
| 1-heptadecanoyl-glycero-3- | 6.6 | Quant | 510.2 (unit) | 184.0 (unit) | 26.5** | 3 | 120 | 25 | 5 | Positive |
| phosphocholine | | Qual | 510.2 (unit) | 86.1 (unit) | | 3 | 120 | 45 | 5 | Positive |
| Eicosapentaenoic acid | 6.6 | Quant | 301.1 (Unit) | 257.0 (Unit) | 15.1 | 3 | 120 | 5 | 5 | Negative |
| | | Qual | 301.1 (Unit) | 59.2 (Unit) | | 3 | 120 | 15 | 5 | Negative |
| Bilirubin | 6.5 | Quant | 585.2 (Unit) | 299.1 (Unit) | 0.7 | 3 | 125 | 20 | 5 | Positive |
| | | Qual | 585.2 (Unit) | 213.1 (Unit) | | 3 | 125 | 45 | 3 | Positive |
| Biliverdin | 5.1 | Quant | 583.2 (Unit) | 297.1 (Unit) | 200.7 | 3 | 135 | 35 | 5 | Positive |
| | | Qual | 583.2 (Unit) | 583.1 (Unit) | | 3 | 135 | 0 | 5 | Positive |
| Etiocholanolone glucuronide | 7.1 | Quant | 465.2 (Unit) | 465.1 (Unit) | Na | 3 | 135 | 0 | 6 | Negative |
| | | Qual | 465.2 (Unit) | 113.0 (Unit) | | 3 | 135 | 35 | 6 | Negative |
| Myristic acid | 6.7 | Quant | 227.2 (Unit) | 227.1 (Unit) | 0.5 | 3 | 145 | 0 | 4 | Negative |
| | | Qual | 227.2 (Unit) | 53.8 (Unit) | | 3 | 145 | 45 | 4 | Negative |
| Stearic acid | 7.4 | Quant | 283.2 (Unit) | 265.0 (Unit) | 6.8 | 3 | 145 | 19 | 2 | Negative |
| | | Qual | 283.2 (Unit) | 45.1 (Unit) | | 3 | 145 | 20 | 2 | Negative |
| 1-Palmitoy1-2-hydroxy-sn-glycero-3- | 6.5 | Quant | 496.2 (Unit) | 104.1 (Unit) | 34.3 | 3 | 120 | 10 | 2 | Positive |
| phosphocholine (LysoPC(16:0)) | | Qual | 496.2 (Unit) | 184.0 (Unit) | | 3 | 120 | 5 | 2 | Positive |

SIL-IS

| | | | | | | | | | | |
|---|---|---|---|---|---|---|---|---|---|---|
| 25-Hydroxyvitamin D3-[²H₃] | 6.6 | Quant | 404.2 (Wide) | 386.2 (Wide) | 46.6 | 20 | 98 | 10 | 2 | Positive |
| | | Qual | 404.2 (Wide) | 368.3 (Wide) | | 20 | 98 | 10 | 2 | Positive |
| Arachidonic acid-[²H₈] | 6.8 | Quant | 311.1 (Unit) | 267.1 (Unit) | 36.1 | 3 | 135 | 3 | 2 | Negative |
| | | Qual | 311.1 (Unit) | 59.1 (Unit) | | 3 | 135 | 15 | 3 | Negative |
| Decanoylcarnitine-[²H₃] | 4.7 | Quant | 319.1 (Unit) | 63.1 (Unit) | 46.6 | 3 | 190 | 24 | 2 | Positive |
| | | Qual | 319.1 (Unit) | 257.1 (Unit) | | 3 | 190 | 12 | 2 | Positive |
| Dodecanoyl-l-carnitine-[²H₃] | 5.4 | Quant | 347.1 (Unit) | 85.1 (Unit) | 29.8 | 3 | 140 | 21 | 3 | Positive |
| | | Qual | 347.1 (Unit) | 85.0 (Unit) | | 3 | 140 | 51 | 3 | Positive |
| Docosahexaenoic acid-[²H₅] | 6.7 | Quant | 332.1 (Unit) | 288.1 (Unit) | 12.1 | 3 | 80 | 5 | 2 | Negative |
| | | Qual | 332.1 (Unit) | 234.1 (Unit) | | 3 | 80 | 5 | 2 | Negative |
| 1,3-Dilinoleoyl-rac-glycerol-[²H₅] | 8.8 | Quant | 639.4 (Unit) | 342.5 (Unit) | 141.1 | 3 | 84 | 20 | 2 | Positive |
| | | Qual | 639.4 (Unit) | 604.2 (Unit) | | 3 | 84 | 10 | 2 | Positive |
| Hexadecanoic acid-[²H₄] | 7.0 | Quant | 259.1 (Unit) | 259.1 (Unit) | 24.1 | 3 | 130 | 15 | 3 | Negative |
| | | Qual | 259.1 (Unit) | 259.0 (Unit) | | 3 | 130 | 20 | 3 | Negative |
| Linoleic acid-[¹³C₁₈] | 6.9 | Quant | 297.3 (Unit) | 297.3 (Unit) | 21.5 | 3 | 104 | 10 | 3 | Negative |
| | | Qual | 297.3 (Unit) | 297.2 (Unit) | | 3 | 104 | 20 | 3 | Negative |
| Oleic acid-[¹³C₅] | 7.1 | Quant | 286.3 (Unit) | 286.3 (Unit) | 24.5 | 3 | 128 | 10 | 3 | Negative |
| | | Qual | 286.3 (Unit) | 286.2 (Unit) | | 3 | 128 | 20 | 3 | Negative |
| Palmitoyl carnitine-[²H₃] | 6.3 | Quant | 403.2 (Unit) | 63.2 (Unit) | 29.7 | 3 | 190 | 5 | 2 | Positive |
| | | Qual | 403.2 (Unit) | 341.2 (Unit) | | 3 | 190 | 0 | 2 | Positive |
| Sphingosine-1-phosphate-[¹³C₂,²H₂] | 6.1 | Quant | 384.2 (Unit) | 268.2 (Unit) | 4.3 | 3 | 100 | 11 | 3 | Positive |
| | | Qual | 384.2 (Unit) | 366.2 (Unit) | | 3 | 100 | 11 | 3 | Positive |
| Stearoyl-L-carnitine [²H₃] | 6.7 | Quant | 431.3 (Unit) | 85.0 (Unit) | 2.7 | 3 | 130 | 25 | 5 | Positive |
| | | Qual | 431.3 (Unit) | 369.3 (Unit) | | 3 | 130 | 15 | 5 | Positive |
| Bilirubin [²H₄] | 6.5 | Quant | 590.2 (Unit) | 301.2 (Unit) | 98.8 | 3 | 125 | 15 | 5 | Positive |
| | | Qual | 590.2 (Unit) | 303.2 (Unit) | | 3 | 125 | 15 | 5 | Positive |
| Biliverdin [²H₄] | 5.1 | Quant | 586.0 (Unit) | 299.2 (Unit) | NA | 3 | 130 | 35 | 5 | Positive |

⁺read-out is a combined signal of 1,3-rac-Dilinoleoyl-glycerol and 1,2-rac-Dilinoleoyl-glycerol;
**read-out is the signal of total Octadecenoic acid, constituting mainly oleic acid.

TABLE 5
LC-MRM parameters for the hydrophilic metabolites of interest and associated SIL-IS

| | Rt | Quant/ | MS1 | MS2 | Quant/ | Dwell | Frag | CE | CAV | |
|---|---|---|---|---|---|---|---|---|---|---|
| Metabolite | (min) | Qual | m/z (Res) | m/z (Res) | Qual ratio | (ms) | (V) | (V) | (V) | Polarity |
| 2-Hydroxybutanoic acid | 2.3 | Quant | 103.0 (Unit) | 57.2 (Unit) | 14.0 | 15 | 84 | 8 | 4 | Negative |
| | | Qual | 103.0 (Unit) | 45.2 (Unit) | | 15 | 84 | 5 | 4 | Negative |
| 3-Hydroxybutanoic acid | 2.7 | Quant | 103.1 (Unit) | 59.1 (Unit) | 344.9 | 15 | 78 | 8 | 4 | Negative |
| | | Qual | 103.1 (Unit) | 103.1 (Unit) | | 15 | 78 | 0 | 4 | Negative |
| L-Alanine | 4.6 | Quant | 90.1 (Unit) | 90.1 (Unit) | 74.7 | 3 | 62 | 0 | 2 | Positive |
| | | Qual | 90.1 (Unit) | 44.1 1 (Unit) | | 3 | 62 | 8 | 2 | Positive |
| L-Arginine | 6.8 | Quant | 175.0 (Unit) | 116.0 (Unit) | 505.9 | 3 | 82 | 15 | 2 | Positive |
| | | Qual | 175.0 (Unit) | 70.1 (Unit) | | 3 | 82 | 20 | 2 | Positive |
| L-Leucine | 3.4 | Quant | 132.0 (Unit) | 86.2 (Unit) | 19.6 | 3 | 104 | 10 | 4 | Positive |
| | | Qual | 132.0 (Unit) | 44.2 (Unit) | | 3 | 104 | 25 | 4 | Positive |
| Citrulline | 5.0 | Quant | 176.0 (Unit) | 113.0 (Unit) | 261.2 | 3 | 68 | 15 | 5 | Positive |
| | | Qual | 176.0 (Unit) | 70.1 (Unit) | | 3 | 68 | 20 | 5 | Positive |
| Choline | 5.7 | Quant | 104.1 (Unit) | 45.3 (Unit) | 171.5 | 3 | 40 | 27 | 2 | Positive |
| | | Qual | 104.1 (Unit) | 60.3 (Unit) | | 3 | 40 | 17 | 2 | Positive |
| Glycyl-glycine | 5.4 | Quant | 133.1 (Unit) | 30.4 (Unit) | 15.7 | 3 | 58 | 20 | 4 | Positive |
| | | Qual | 133.1 (Unit) | 76.2 (Unit) | | 3 | 58 | 5 | 4 | Positive |
| Homo-L-arginine | 6.8 | Quant | 189.0 (Unit) | 144.2 (Unit) | 2.6 | 3 | 88 | 15 | 2 | Positive |
| | | Qual | 189.0 (Unit) | 57.1 (Unit) | | 3 | 88 | 25 | 2 | Positive |
| L-Isoleucine | 3.6 | Quant | 132.0 (Unit) | 69.2 (Unit) | 22.9 | 3 | 104 | 19 | 2 | Positive |
| | | Qual | 132.0 (Unit) | 57.2 (Unit) | | 3 | 104 | 32 | 2 | Positive |
| L-Methionine | 3.5 | Quant | 150.0 (Unit) | 56.2 (Unit) | 37.0 | 3 | 104 | 16 | 2 | Positive |
| | | Qual | 150.0 (Unit) | 104.1 (Unit) | | 3 | 104 | 14 | 2 | Positive |
| NG-Monomethyl-L-arginine | 7.2 | Quant | 189.0 (Unit) | 116.2 (Unit) | 180.2 | 3 | 88 | 15 | 2 | Positive |
| | | Qual | 189.0 (Unit) | 70.2 (Unit) | | 3 | 88 | 15 | 2 | Positive |
| Asymmetric dimethylarginine | 7.9 | Quant | 203.0 (Unit) | 46.2 (Unit) | 188.5 | 3 | 100 | 15 | 4 | Positive |
| | | Qual | 203.0 (Unit) | 70.1 (Unit) | | 3 | 100 | 18 | 4 | Positive |
| Symmetric dimethylarginine | 7.7 | Quant | 203.1 (Unit) | 172.2 (Unit) | 49.3 | 3 | 90 | 10 | 4 | Positive |
| | | Qual | 203.1 (Unit) | 133.0 (Unit) | | 3 | 90 | 6 | 4 | Positive |
| Taurine | 2.9 | Quant | 126.1 (Unit) | 44.2 (Unit) | 35.7 | 3 | 100 | 20 | 2 | Positive |
| | | Qual | 126.1 (Unit) | 108.0 (Unit) | | 3 | 100 | 10 | 2 | Positive |
| Urea | 1.3 | Quant | 61.2 (Unit) | 44.3 (Unit) | 167.3 | 3 | 100 | 10 | 2 | Positive |
| | | Qual | 61.2 (Unit) | 61.2 (Unit) | | 3 | 100 | 10 | 2 | Positive |
| Cotinine | 1.3 | Quant | 177.0 (Unit) | 80.0 (Unit) | 19.7 | 3 | 100 | 25 | 5 | Positive |
| | | Qual | 177.0 (Unit) | 98.1 (Unit) | | 3 | 100 | 20 | 5 | Positive |
| L-(+)-Ergothioneine | 4.8 | Quant | 230.1 (Unit) | 127.0 (Unit) | 47.3 | 3 | 100 | 25 | 2 | Positive |
| | | Qual | 230.1 (Unit) | 186.0 (Unit) | | 3 | 100 | 15 | 2 | Positive |
| L-Acetylcarnitine | 6.1 | Quant | 204.2 (Unit) | 60.1 (Unit) | 377.5 | 3 | 100 | 15 | 4 | Positive |
| | | Qual | 204.2 (Unit) | 85.0 (Unit) | | 3 | 100 | 15 | 4 | Positive |
| L-Lysine | 7.4 | Quant | 146.9 (Unit) | 130.2 (Unit) | 613.4 | 1 | 100 | 20 | 2 | Positive |
| | | Qual | 146.9 (Unit) | 84.2 (Unit) | | 1 | 100 | 2 | 2 | Positive |
| L-Glutamine | 4.8 | Quant | 144.9 (Unit) | 127.0 (Unit) | 31.8 | 3 | 100 | 10 | 2 | Negative |
| | | Qual | 144.9 (Unit) | 108.8 (Unit) | | 3 | 100 | 15 | 2 | Negative |

SIL-IS

| | | | | | | | | | | |
|---|---|---|---|---|---|---|---|---|---|---|
| 2-Hydroxybutyrate-[²H₃] | 2.5 | Quant | 106.0 (Unit) | 59.2 (Unit) | 12.9 | 15 | 84 | 8 | 4 | Negative |
| | | Qual | 106.0 (Unit) | 45.2 (Unit) | | 15 | 84 | 5 | 4 | Negative |
| 3-Hydroxybutanoic acid [²H₄] | 2.7 | Quant | 107.0 (Unit) | 107.0 (Unit) | 26.7 | 15 | 78 | 0 | 3 | Negative |
| | | Qual | 107.0 (Unit) | 59.1 (Unit) | | 15 | 78 | 8 | 3 | Negative |
| L-Alanine-[¹³C₃] | 4.6 | Quant | 93.1 (Unit) | 93.1 (Unit) | 94.5 | 3 | 62 | 0 | 2 | Positive |
| | | Qual | 93.1 (Unit) | 46.1 (Unit) | | 3 | 62 | 8 | 2 | Positive |
| L-Arginine-[¹³C₆] | 6.8 | Quant | 181.2 (Unit) | 61.3 (Unit) | 55.6 | 3 | 82 | 12 | 2 | Positive |
| | | Qual | 181.2 (Unit) | 121.1 (Unit) | | 3 | 82 | 12 | 2 | Positive |
| Leucine-[¹³C₆] | 3.4 | Quant | 138.0 (Unit) | 46.2 (Unit) | 10.7 | 3 | 104 | 25 | 4 | Positive |
| | | Qual | 138.0 (Unit) | 44.2 (Unit) | | 3 | 104 | 25 | 4 | Positive |
| L-Citrulline [²H₇] | 5.1 | Quant | 183.1 (Unit) | 120.1 (Unit) | 231.6 | 3 | 68 | 16 | 5 | Positive |
| | | Qual | 183.1 (Unit) | 166.1 (Unit) | | 3 | 68 | 4 | 5 | Positive |
| Choline-[²H₉] | 5.7 | Quant | 114.0 (Unit) | 45.2 (Unit) | 141.3 | 3 | 40 | 20 | 2 | Positive |
| | | Qual | 114.0 (Unit) | 69.2 (Unit) | | 3 | 40 | 20 | 2 | Positive |
| Glycyl-glycine [¹³C₄, ¹⁵N₂] | 5.4 | Quant | 138.9 (Unit) | 79.1 (Unit) | 24.5 | 3 | 58 | 5 | 4 | Positive |
| | | Qual | 138.9 (Unit) | 32.2 (Unit) | | 3 | 58 | 20 | 4 | Positive |
| Homo-L-arginine [¹³C₇, ¹⁵N₄] | 6.8 | Quant | 200.0 (Unit) | 153.0 (Unit) | 717.1 | 3 | 88 | 5 | 2 | Positive |
| | | Qual | 200.0 (Unit) | 90.2 (Unit) | | 3 | 88 | 20 | 2 | Positive |
| Isoleucine-[¹³C₆] | 3.6 | Quant | 138.0 (Unit) | 74.2 (Unit) | 24.6 | 3 | 104 | 19 | 3 | Positive |
| | | Qual | 138.0 (Unit) | 60.2 (Unit) | | 3 | 104 | 32 | 3 | Positive |
| L-Methionine-[¹³C₅] | 3.5 | Quant | 155.0 (Unit) | 59.2 (Unit) | 40.0 | 3 | 104 | 16 | 2 | Positive |
| | | Qual | 155.0 (Unit) | 108.2 (Unit) | | 3 | 104 | 14 | 2 | Positive |
| Asymmetric dimethylarginine [²H₆] | 7.9 | Quant | 209.2 (Unit) | 52.3 (Unit) | 175.0 | 3 | 100 | 15 | 4 | Positive |
| | | Qual | 209.2 (Unit) | 70.2 (Unit) | | 3 | 100 | 20 | 4 | Positive |
| Symmetric Dimethylarginine- [²H₆] | 7.7 | Quant | 209.1 (Unit) | 175.1 (Unit) | 41.9 | 3 | 90 | 10 | 4 | Positive |
| | | Qual | 209.1 (Unit) | 164.0 (Unit) | | 3 | 90 | 15 | 4 | Positive |
| Taurine [¹³C₂] | 2.9 | Quant | 128.1 (Unit) | 46.2 (Unit) | 74.2 | 3 | 102 | 16 | 3 | Positive |
| | | Qual | 128.1 (Unit) | 110.2 (Unit) | | 3 | 102 | 8 | 3 | Positive |
| Urea [¹³C, ¹⁸O] | 1.3 | Quant | 64.2 (Unit) | 47.2 (Unit) | 469.3 | 3 | 100 | 25 | 2 | Positive |
| | | Qual | 64.2 (Unit) | 64.1 (Unit) | | 3 | 100 | 0 | 2 | Positive |
| (±)-Cotinine [²H₃] | 1.4 | Quant | 180.0 (Unit) | 80.0 (Unit) | 21.7 | 3 | 100 | 25 | 5 | Positive |
| | | Qual | 180.0 (Unit) | 101.0 (Unit) | | 3 | 100 | 20 | 5 | Positive |
| L-(+)-Ergothioneine [²H₉] | 4.8 | Quant | 239.0 (Unit) | 127.0 (Unit) | 122.3 | 3 | 100 | 25 | 4 | Positive |
| | | Qual | 239.0 (Unit) | 195.0 (Unit) | | 3 | 100 | 10 | 2 | Positive |
| L-Acetylcarnitine [²H₃] | 6.1 | Quant | 207.2 (Unit) | 60.1 (Unit) | 853.4 | 3 | 100 | 15 | 4 | Positive |
| | | Qual | 207.2 (Unit) | 85.0 (Unit) | | 3 | 100 | 15 | 4 | Positive |
| L-Glutamine [¹³C₅] | 4.7 | Quant | 149.9 (Unit) | 131.9 (Unit) | 24.8 | 3 | 100 | 10 | 2 | Negative |
| | | Qual | 149.9 (Unit) | 113.8 (Unit) | | 3 | 100 | 15 | 2 | Negative |

^{#}in source fragmentation

TABLE 6
Metabolites considered in the discovery of univariable and multivariable predictors of PE in the obese pregnant women

| | | | Intra-day | |
|---|---|---|---|---|
| Metabolite | SIL-IS | Quantifier | normalisation | % CV |
| 25-Hydroxyvitamin D₃ | 25-Hydroxyvitamin D3-[²H₃] | anlt-ISTD_Resp._Ratio | Yes | 17% |
| 2-Hydroxybutanoic acid | 2-Hydroxybutyrate-[²H₃] | anlt-ISTD_Resp._Ratio | No | 5% |
| 3-Hydroxybutanoic acid | 3-Hydroxybutanoic acid [²H₄] | anlt-ISTD_Resp._Ratio | No | 4% |
| L-alanine | L-Alanine-[¹³C₃] | anlt-ISTD_Resp._Ratio | Yes | 7% |
| Arachidonic acid | Arachidonic acid-[²H₈] | anlt-ISTD_Resp._Ratio | Yes | 7% |
| L-arginine | L-Arginine-[¹³C₆] | anlt-ISTD_Resp._Ratio | No | 6% |
| L-leucine | Leucine-[¹³C₆] | anlt-ISTD_Resp._Ratio | No | 4% |
| 8,11,14 Eicosatrienoic acid | Arachidonic acid-[²H₈] | anlt-ISTD_Resp._Ratio | Yes | 6% |
| Citrulline | L-Citrulline [²H₇] | anlt-ISTD_Resp._Ratio | Yes | 7% |
| Decanoylcarnitine | Decanoylcarnitine-[²H₃] | anlt-ISTD_Resp._Ratio | No | 7% |
| Dodecanoyl-l-carnitine (c12) | Dodecanoyl-l-carnitine-[²H₃] | anlt-ISTD_Resp._Ratio | No | 5% |
| Docosahexaenoic acid | Docosahexaenoic acid-[²H₅] | anlt-ISTD_Resp._Ratio | No | 5% |
| Dilinoleoyl-glycerol | 1,3-Dilinoleoyl-rac-glycerol-[²H₅] | anlt-ISTD_Resp._Ratio | Yes | 9% |
| Choline | Choline-[²H₉] | anlt-ISTD_Resp._Ratio | No | 4% |
| Glycyl-glycine | Glycyl-glycine [¹³C₄, ¹⁵N₂] | anlt-ISTD_Resp._Ratio | Yes | 24% |
| Homo-L-arginine | Homo-L-arginine [¹³C₇, ¹⁵N₄] | anlt-ISTD_Resp._Ratio | No | 4% |
| Hexadecanoic acid | Hexadecanoic acid-[²H₄] | anlt-ISTD_Resp._Ratio | No | 7% |
| L-Isoleucine | Isoleucine-[¹³C₆] | anlt-ISTD_Resp._Ratio | No | 4% |
| Linoleic acid | Linoleic acid-[¹³C₁₈] | qual-ISTD_Resp._Ratio | No | 5% |
| L-methionine | L-Methionine-[¹³C₅] | anlt-ISTD_Resp._Ratio | No | 5% |
| NG-Monomethyl-L-arginine | Homo-L-arginine [¹³C₇, ¹⁵N₄] | anlt-ISTD_Resp._Ratio | Yes | 9% |
| Octadecenoic acid | Oleic acid-[¹³C₅] | qual-ISTD_Resp._Ratio | No | 5% |
| L-Palmitoylcarnitine | Palmitoyl carnitine-[²H₃] | anlt-ISTD_Resp._Ratio | No | 10% |
| Asymmetric dimethylarginine | Asymmetric dimethylarginine [²H₆] | anlt-ISTD_Resp._Ratio | No | 5% |
| Sphingosine-1-phosphate | Sphingosine-1-phosphate-[¹³C₂, | anlt-ISTD_Resp._Ratio | No | 5% |
| | ²H₂] | | | |
| Sphinganine-1-phosphate | Sphingosine-1-phosphate-[¹³C₂, | anlt-ISTD_Resp._Ratio | No | 9% |
| | ²H₂] | | | |
| Symmetric dimethylarginine | Symmetric Dimethylarginine- [²H₆] | anlt-ISTD_Resp._Ratio | No | 6% |
| Taurine | Taurine [¹³C₂] | anlt-ISTD_Resp._Ratio | No | 5% |
| Urea | Urea [¹³C, ¹⁸O] | anlt-ISTD_Resp._Ratio | No | 4% |
| Stearoylcarnitine | Stearoyl-L-carnitine [²H₃] | anlt-ISTD_Resp._Ratio | No | 9% |
| Eicosapentaenoic acid | Hexadecanoic acid-[²H₄] | anlt-ISTD_Resp._Ratio | Yes | 18% |
| 1-heptadecanoyl-2-hydroxy-sn- | Sphingosine-1-phosphate-[¹³C₂, | anlt-ISTD_Resp._Ratio | Yes | 7% |
| glycero-3-phosphocholine | ²H₂] | | | |
| Bilirubin | Bilirubin [²H₄] | anlt-ISTD_Resp._Ratio | Yes | 9% |
| Biliverdin | Biliverdin [²H₄] | anlt-ISTD_Resp._Ratio | Yes | 15% |
| Etiocholanolone glucuronide | Oleic acid-[¹³C₅] | anlt-ISTD_Resp._Ratio | Yes | 11% |
| Cotinine | (±)-Cotinine [²H₃] | anlt-ISTD_Resp._Ratio | No | 26% |
| Myristic acid | Docosahexaenoic acid-[²H₅] | anlt-ISTD_Resp._Ratio | Yes | 9% |
| Stearic acid | Linoleic acid-[¹³C₁₈] | anlt-ISTD_Resp._Ratio | No | 11% |
| L-(+)-Ergothioneine | L-(+)-Ergothioneine [²H₉] | anlt-ISTD_Resp._Ratio | Yes | 15% |
| Met_XXX | Docosahexaenoic acid-[²H₅] | anlt-Area | Yes | 8% |
| 1-Palmitoyl-2-hydroxy-sn- | Linoleic acid-[¹³C₁₈] | qual-ISTD_Resp._Ratio | Yes | 6% |
| glycero-3-phosphocholine | | | | |
| L-Acetylcarnitine | L-Acetylcarnitine [²H₃] | anlt-ISTD_Resp._Ratio | No | 4% |
| L-Lysine | L-Acetylcarnitine [²H₃] | anlt-ISTD_Resp._Ratio | Yes | 6% |
| L-Glutamine | L-Glutamine [¹³C₅] | anlt-ISTD_Resp._Ratio | No | 5% |

TABLE 8
Single predictors for PE in the obese; stratum: all; outcome: Total PE

| | Prediction of PE in the obese | | | | |
|---|---|---|---|---|---|
| | (stratum: all/outcome: total PE) | | | | |
| | | AUC | AUC | Δ cases:con- | controls |
| Predictors | AUC | lCI 95% CI | uCI 95% CI | trols | (n):cases (n) |
| Mat_dbp_m | 0.68 | 0.61 | 0.75 | UP | 633:63 |
| Mat_sbp_m | 0.67 | 0.60 | 0.74 | UP | 633:63 |
| NG-Monomethyl-L-arginine* | 0.65 | 0.57 | 0.72 | DOWN | 645:65 |
| Biliverdin | 0.62 | 0.55 | 0.69 | DOWN | 643:65 |
| Glycyl-glycine | 0.62 | 0.54 | 0.70 | DOWN | 645:65 |
| Dilinoleoyl-glycerol | 0.61 | 0.54 | 0.68 | UP | 645:65 |
| Etiocholanolone glucuronide | 0.61 | 0.54 | 0.69 | UP | 645:65 |
| Bilirubin | 0.62 | 0.54 | 0.70 | DOWN | 645:65 |
| L-leucine | 0.60 | 0.53 | 0.67 | UP | 645:65 |
| Mat_trig | 0.60 | 0.53 | 0.67 | UP | 640:65 |
| Mat_prev_pet | 0.60 | 0.52 | 0.69 | n/a | 362:29 |
| PIGF | 0.60 | 0.52 | 0.67 | DOWN | 645:65 |
| 25-Hydroxyvitamin D3 | 0.59 | 0.52 | 0.67 | DOWN | 568:59 |
| 1-heptadecanoyl-2-hydroxy- | 0.59 | 0.51 | 0.66 | DOWN | 645:65 |
| sn-glycero-3-phosphocholine | | | | | |
| L-(+)-Ergothioneine | 0.58 | 0.51 | 0.66 | DOWN | 645:65 |
| Octadecenoic acid | 0.58 | 0.51 | 0.65 | UP | 645:65 |
| Met_XXX | 0.59 | 0.51 | 0.67 | DOWN | 645:65 |
| Mat_bmi | 0.59 | 0.51 | 0.67 | UP | 645:65 |
| Hexadecanoic acid | 0.58 | 0.51 | 0.65 | UP | 645:65 |
| Mat_hdl | 0.57 | 0.50 | 0.63 | DOWN | 640:65 |
| Linoleic acid | 0.57 | 0.50 | 0.65 | UP | 645:65 |
| L-Isoleucine | 0.57 | 0.50 | 0.64 | UP | 645:65 |

TABLE 9
Single predictors for total PE in the obese; delineation in accordance to developing the comorbidity GDM.

| | | | | | | Prediction of PE in the obese | | |
|---|---|---|---|---|---|---|---|---|
| | Prediction of PE in the obese who will not | | | | | who will develop | | |
| | develop GDM (stratum:no GDM) | | | | | GDM (stratum: GDM) | | |
| | | AUC ICI | AUC uCI | Δ cases: | controls(n): | | AUC ICI | AUC uCI |
| Predictors | AUC | 95% CI | 95% CI | controls | cases(n) | AUC | 95% CI | 95% CI |
| Mat_dbp_m | 0.70 | 0.62 | 0.78 | UP | 458:43 | 0.66 | 0.51 | 0.80 |
| Mat_sbp_m | 0.64 | 0.56 | 0.73 | UP | 458:43 | 0.76 | 0.64 | 0.88 |
| NG-Monomethyl-L-arginine* | 0.70 | 0.61 | 0.78 | DOWN | 465:45 | 0.55 | 0.41 | 0.69 |
| Biliverdin | 0.64 | 0.55 | 0.72 | DOWN | 463:45 | 0.60 | 0.44 | 0.75 |
| Glycyl-glycine* | 0.61 | 0.52 | 0.71 | DOWN | 465:45 | 0.67 | 0.53 | 0.81 |
| Dilinoleoyl-glycerol | 0.65 | 0.57 | 0.73 | UP | 465:45 | 0.48 | 0.34 | 0.62 |
| Etiocholanolone glucuronide | 0.63 | 0.54 | 0.72 | UP | 465:45 | 0.59 | 0.43 | 0.75 |
| Bilirubin | 0.62 | 0.52 | 0.72 | DOWN | 465:45 | 0.65 | 0.49 | 0.81 |
| L-Ieucine | 0.61 | 0.52 | 0.70 | UP | 465:45 | 0.56 | 0.43 | 0.68 |
| Mat_trig | 0.63 | 0.55 | 0.71 | UP | 460:45 | 0.52 | 0.36 | 0.69 |
| Mat_prev_pet | 0.62 | 0.52 | 0.72 | n/a | 254:20 | 0.59 | 0.42 | 0.75 |
| PIGF | 0.63 | 0.55 | 0.71 | DOWN | 465:45 | 0.52 | 0.35 | 0.69 |
| 25-Hydroxyvitamin D3 | 0.57 | 0.48 | 0.66 | | 412.41 | 0.62 | 0.46 | 0.78 |
| 1-heptadecanoy1-2-hydroxy-sn- | 0.57 | 0.48 | 0.66 | | 465:45 | 0.59 | 0.46 | 0.71 |
| glycero-3-phosphocholine | | | | | | | | |
| L-(+)-Ergothioneine | 0.58 | 0.49 | 0.67 | | 465:45 | 0.60 | 0.46 | 0.71 |
| Octadecenoic acid | 0.59 | 0.51 | 0.68 | UP | 465:45 | 0.47 | 0.31 | 0.62 |
| Met_XXX | 0.61 | 0.51 | 0.70 | DOWN | 465:45 | 0.56 | 0.39 | 0.73 |
| Mat_bmi | 0.57 | 0.48 | 0.67 | | 465:45 | 0.61 | 0.44 | 0.77 |
| Hexadecanoic acid | 0.59 | 0.51 | 0.68 | UP | 465:45 | 0.48 | 0.31 | 0.64 |
| Mat_hdl | 0.56 | 0.48 | 0.65 | | 460:45 | 0.57 | 0.44 | 0.70 |
| Linoleic acid | 0.60 | 0.51 | 0.68 | UP | 465:45 | 0.49 | 0.32 | 0.65 |
| L- Isoleucine | 0.58 | 0.49 | 0.67 | | 465:45 | 0.53 | 0.40 | 0.66 |
| Stearic acid | 0.58 | 0.50 | 0.66 | UP | 465:45 | 0.50 | 0.33 | 0.67 |
| Asymmetric dimethylarginine | 0.52 | 0.43 | 0.61 | | 465:45 | 0.66 | 0.53 | 0.78 |
| Taurine | 0.53 | 0.45 | 0.61 | | 465:45 | 0.65 | 0.52 | 0.78 |

| | Prediction of PE in the | | | | | | | |
|---|---|---|---|---|---|---|---|---|
| | obese who will develop | | | | | | | |
| | GDM (stratum: GDM) | | Δ AUC: | Prediction of PE in the obese (stratum:all) | | | | |
| | Δ cases: | controls(n): | no GDM − | | AUC ICI | AUC uCI | Δ cases: | controls(n): |
| Predictors | controls | cases(n) | GDM | AUC | 95% CI | 95% CI | controls | cases(n) |
| Mat_dbp_m | UP | 164:18 | 0.04 | 0.68 | 0.61 | 0.75 | UP | 633:63 |
| Mat_sbp_m | UP | 164:18 | −0.12 | 0.67 | 0.60 | 0.74 | UP | 633:63 |
| NG-Monomethyl-L-arginine* | | 169:18 | 0.15 | 0.65 | 0.57 | 0.72 | DOWN | 645:65 |
| Biliverdin | | 169:18 | 0.04 | 0.62 | 0.55 | 0.69 | DOWN | 643:65 |
| Glycyl-glycine* | DOWN | 169:18 | −0.06 | 0.62 | 0.54 | 0.70 | DOWN | 645:65 |
| Dilinoleoyl-glycerol | | 169:18 | 0.17 | 0.61 | 0.54 | 0.68 | UP | 645:65 |
| Etiocholanolone glucuronide | | 169:18 | 0.04 | 0.61 | 0.54 | 0.69 | UP | 645:65 |
| Bilirubin | | 169:18 | −0.03 | 0.62 | 0.54 | 0.70 | DOWN | 645:65 |
| L-Ieucine | | 169:18 | 0.05 | 0.60 | 0.53 | 0.67 | UP | 645:65 |
| Mat_trig | | 169:18 | 0.11 | 0.60 | 0.53 | 0.67 | UP | 640:65 |
| Mat_prev_pet | | 102:8 | 0.03 | 0.60 | 0.52 | 0.69 | n/a | 362:29 |
| PIGF | | 169:18 | 0.11 | 0.60 | 0.52 | 0.67 | DOWN | 645:65 |
| 25-Hydroxyvitamin D3 | | 146.16 | −0.04 | 0.59 | 0.52 | 0.67 | DOWN | 568:59 |
| 1-heptadecanoy1-2-hydroxy-sn- | | 169:18 | −0.02 | 0.59 | 0.51 | 0.66 | DOWN | 645.65 |
| glycero-3-phosphocholine | | | | | | | | |
| L-(+)-Ergothioneine | | 169:18 | −0.02 | 0.58 | 0.51 | 0.66 | DOWN | 645:65 |
| Octadecenoic acid | | 169:18 | 0.13 | 0.58 | 0.51 | 0.65 | UP | 645:65 |
| Met_XXX | | 169:18 | 0.04 | 0.59 | 0.51 | 0.67 | DOWN | 645:65 |
| Mat_bmi | | 169:18 | −0.03 | 0.59 | 0.51 | 0.67 | UP | 645:65 |
| Hexadecanoic acid | | 169:18 | 0.12 | 0.58 | 0.51 | 0.65 | UP | 645:65 |
| Mat_hdl | | 169:18 | 0.00 | 0.57 | 0.50 | 0.63 | DOWN | 640:65 |
| Linoleic acid | | 169:18 | 0.11 | 0.57 | 0.50 | 0.65 | UP | 645:65 |
| L- Isoleucine | | 169:18 | 0.05 | 0.57 | 0.50 | 0.64 | UP | 645:65 |
| Stearic acid | | 169:18 | 0.08 | 0.56 | 0.48 | 0.63 | | 645:65 |
| Asymmetric dimethylarginine | UP | 169:18 | −0.14 | 0.54 | 0.46 | 0.61 | | 645:65 |
| Taurine | UP | 169:18 | −0.12 | 0.53 | 0.46 | 0.60 | | 645:65 |

TABLE 10
Single predictors for total PE in the obese; delineation in accordance to parity (1st time pregnant vs.
2nd and more time pregnant)

| | | | | | | Prediction of PE in the | | |
|---|---|---|---|---|---|---|---|---|
| | | | | | | 2nd or more time | | |
| | Prediction of PE in the 1st time pregnant | | | | | pregnant obese women | | |
| | obese women (stratum:nulliparous) | | | | | (stratum:multiparous) | | |
| | | AUC ICI | AUC uCI | Δ cases: | controls(n): | | AUC ICI | AUC uCI |
| Predictors | AUC | 95% CI | 95% CI | controls | cases(n) | AUC | 95% CI | 95% CI |
| Mat_dbp_m | 0.62 | 0.52 | 0.72 | UP | 280:38 | 0.75 | 0.65 | 0.85 |
| Mat_sbp_m | 0.62 | 0.53 | 0.72 | UP | 280:38 | 0.71 | 0.61 | 0.81 |
| NG-Monomethyl-L-arginine | 0.68 | 0.59 | 0.77 | DOWN | 287:38 | 0.59 | 0.46 | 0.72 |
| Biliverdin* | 0.67 | 0.57 | 0.77 | DOWN | 287:38 | 0.57 | 0.46 | 0.68 |
| Glycyl-glycine* | 0.59 | 0.49 | 0.69 | | 287:38 | 0.65 | 0.53 | 0.77 |
| Dilinoleoyl-glycerol | 0.62 | 0.53 | 0.72 | UP | 287:38 | 0.61 | 0.50 | 0.71 |
| Etiocholanolone glucuronide | 0.60 | 0.50 | 0.70 | UP | 287:38 | 0.63 | 0.51 | 0.75 |
| Bilirubin | 0.61 | 0.50 | 0.73 | DOWN | 287:38 | 0.63 | 0.51 | 0.74 |
| L-Ieucine | 0.62 | 0.53 | 0.70 | UP | 287:38 | 0.60 | 0.48 | 0.72 |
| Mat_trig | 0.61 | 0.52 | 0.71 | UP | 283:38 | 0.59 | 0.49 | 0.69 |
| Mat_prev pet | | | | | | 0.61 | 0.53 | 0.70 |
| PIGF | 0.58 | 0.48 | | DOWN | 287:38 | 0.60 | 0.48 | 0.73 |
| 25-Hydroxyvitamin D3 | 0.58 | 0.47 | | DOWN | 252:34 | 0.62 | 0.52 | 0.72 |
| 1-heptadecanoy1-2-hydroxy-sn- | 0.59 | 0.49 | | DOWN | 287:38 | 0.60 | 0.48 | 0.71 |
| glycero-3-phosphocholine | | | | | | | | |
| L-(+)-Ergothioneine | 0.58 | 0.48 | | DOWN | 287:38 | 0.61 | 0.50 | 0.72 |
| Octadecenoic acid | 0.57 | 0.48 | | UP | 287:38 | 0.58 | 0.47 | 0.69 |
| Met_XXX | 0.56 | 0.45 | | DOWN | 287:38 | 0.62 | 0.49 | 0.74 |
| Mat_bmi | 0.60 | 0.50 | 0.71 | UP | 287:38 | 0.57 | 0.44 | 0.69 |
| Hexadecanoic acid | 0.56 | 0.46 | | UP | 287:38 | 0.60 | 0.49 | 0.70 |
| Mat_hdl | 0.58 | 0.49 | | DOWN | 283:38 | 0.54 | 0.44 | 0.63 |
| Linoleic acid | 0.57 | 0.47 | | UP | 287:38 | 0.59 | 0.48 | 0.70 |
| L- Isoleucine | 0.60 | 0.51 | 0.70 | UP | 287:38 | 0.55 | 0.43 | 0.66 |
| 8,11,14 Eicosatrienoic acid | 0.53 | 0.42 | | UP | 287:38 | 0.61 | 0.50 | 0.72 |
| L-alanine | 0.61 | 0.52 | 0.69 | DOWN | 287:38 | 0.57 | 0.46 | 0.67 |
| | | | Δ AUC: | | | | | |
| | 2nd or more time | | 1st time | | | | | |
| | pregnant obese women | | pregn. − | | | | | |
| | (stratum:multiparous) | | >=2nd | Prediction of PE in the obese (stratum:all) | | | | |
| | Δ cases: | controls(n): | time | | AUC ICI | AUC uCI | Δ cases: | controls (n): |
| Predictors | controls | cases(n) | pregn. | AUC | 95% CI | 95% CI | controls | cases (n) |
| Mat_dbp_m | UP | 353:25 | −0.13 | 0.68 | 0.61 | 0.75 | UP | 633:63 |
| Mat_sbp_m | UP | 353:25 | −0.09 | 0.67 | 0.60 | 0.74 | UP | 633:63 |
| NG-Monomethyl-L-arginine | | 358.27 | 0.09 | 0.65 | 0.57 | 0.72 | DOWN | 645:65 |
| Biliverdin* | | 356:27 | 0.10 | 0.62 | 0.55 | 0.69 | DOWN | 643:65 |
| Glycyl-glycine* | DOWN | 358:27 | −0.07 | 0.62 | 0.54 | 0.70 | DOWN | 645:65 |
| Dilinoleoyl-glycerol | UP | 358:27 | 0.02 | 0.61 | 0.54 | 0.68 | UP | 645:65 |
| Etiocholanolone glucuronide | UP | 358:27 | −0.03 | 0.61 | 0.54 | 0.69 | UP | 645:65 |
| Bilirubin | DOWN | 358:27 | −0.01 | 0.62 | 0.54 | 0.70 | DOWN | 645:65 |
| L-Ieucine | | 358:27 | 0.01 | 0.60 | 0.53 | 0.67 | UP | 645:65 |
| Mat_trig | | 357:27 | 0.02 | 0.60 | 0.53 | 0.67 | UP | 640:65 |
| Mat_prev pet | n/a | 353:27 | 0.60 | 0.52 | 0.69 | | n/a | 362:29 |
| PIGF | | 358:27 | −0.03 | 0.60 | 0.52 | 0.67 | DOWN | 645:65 |
| 25-Hydroxyvitamin D3 | DOWN | 316:25 | −0.04 | 0.59 | 0.52 | 0.67 | DOWN | 568:59 |
| 1-heptadecanoy1-2-hydroxy-sn- | | | −0.01 | 0.59 | 0.51 | 0.66 | DOWN | 645:65 |
| glycero-3-phosphocholine | | 358:27 | | | | | | |
| L-(+)-Ergothioneine | DOWN | 358:27 | −0.03 | 0.58 | 0.51 | 0.66 | DOWN | 645:65 |
| Octadecenoic acid | | 358:27 | −0.01 | 0.58 | 0.51 | 0.65 | UP | 645:65 |
| Met_XXX | | 358:27 | −0.06 | 0.59 | 0.51 | 0.67 | DOWN | 645:65 |
| Mat_bmi | | 358:27 | 0.04 | 0.59 | 0.51 | 0.67 | UP | 645:65 |
| Hexadecanoic acid | | 358:27 | −0.04 | 0.58 | 0.51 | 0.65 | UP | 645:65 |
| Mat_hdl | | 357:27 | 0.05 | 0.57 | 0.50 | 0.63 | DOWN | 640:65 |
| Linoleic acid | | 358:27 | −0.02 | 0.57 | 0.50 | 0.65 | UP | 645:65 |
| L- Isoleucine | | 358:27 | 0.06 | 0.57 | 0.50 | 0.64 | UP | 645:65 |
| 8,11,14 Eicosatrienoic acid | UP | 357:27 | −0.09 | 0.57 | 0.49 | 0.65 | | 644:65 |
| L-alanine | | 358:27 | 0.04 | 0.53 | 0.46 | 0.60 | | 645:65 |

TABLE 11
Single predictors for total PE in the obese; prediction in the morbidly obese (bmi > =40)

| | Prediction of PE in the morbidly obese | | | | | | | | | |
|---|---|---|---|---|---|---|---|---|---|---|
| | (stratum:bmi >40) | | | | | Prediction of PE in the obese (stratum:all) | | | | |
| | | AUC ICI | AUC uCI | Δ cases: | controls (n): | | AUC ICI | AUC uCI | Δ cases: | controls (n): |
| Predictors | AUC | 95% CI | 95% CI | controls | cases (n) | AUC | 95% CI | 95% CI | controls | cases (n) |
| Mat_dbp_m | 0.66 | 0.52 | 0.81 | UP | 106:19 | 0.68 | 0.61 | 0.75 | UP | 633:63 |
| Mat_sbp_m | 0.72 | 0.58 | 0.85 | UP | 106:19 | 0.67 | 0.60 | 0.74 | UP | 633:63 |
| NG-Monomethyl-L-arginine | 0.43 | 0.28 | 0.57 | | 111:20 | 0.65 | 0.57 | 0.72 | DOWN | 645:65 |
| Biliverdin | 0.73 | 0.61 | 0.86 | DOWN | 110:20 | 0.62 | 0.55 | 0.69 | DOWN | 643:65 |
| Glycyl-glycine | 0.60 | 0.46 | 0.74 | | 111:20 | 0.62 | 0.54 | 0.70 | DOWN | 645:65 |
| Dilinoleoyl-glycerol | 0.59 | 0.45 | 0.73 | | 111:20 | 0.61 | 0.54 | 0.68 | UP | 645:65 |
| Etiocholanolone glucuronide | 0.67 | 0.52 | 0.81 | UP | 111:20 | 0.61 | 0.54 | 0.69 | UP | 645:65 |
| Bilirubin* | 0.75 | 0.61 | 0.89 | DOWN | 111:20 | 0.62 | 0.54 | 0.70 | DOWN | 645:65 |
| L-leucine | 0.65 | 0.52 | 0.78 | UP | 111:20 | 0.60 | 0.53 | 0.67 | UP | 645:65 |
| Mat_trig | 0.61 | 0.47 | 0.75 | | 111:20 | 0.60 | 0.53 | 0.67 | UP | 640:65 |
| Mat_prev_pet | 0.57 | 0.43 | 0.72 | | 67:9 | 0.60 | 0.52 | 0.69 | n/a | 362:29 |
| PIGF | 0.55 | 0.40 | 0.70 | | 111:20 | 0.60 | 0.52 | 0.67 | DOWN | 645:65 |
| 25-Hydroxyvitamin D3 | 0.55 | 0.39 | 0.70 | | 98:17 | 0.59 | 0.52 | 0.67 | DOWN | 568:59 |
| 1-heptadecanoy1-2-hydroxy-sn- | 0.56 | 0.42 | 0.71 | | 111:20 | 0.59 | 0.51 | 0.66 | DOWN | 645:65 |
| glycero-3-phosphocholine | | | | | | | | | | |
| L-(+)-Ergothioneine | 0.56 | 0.44 | 0.69 | | 111:20 | 0.58 | 0.51 | 0.66 | DOWN | 645:65 |
| Octadecenoic acid | 0.61 | 0.48 | 0.74 | | 111:20 | 0.58 | 0.51 | 0.65 | UP | 645:65 |
| Met_XXX | 0.53 | 0.38 | 0.68 | | 111:20 | 0.59 | 0.51 | 0.67 | DOWN | 645:65 |
| Mat_bmi | 0.57 | 0.44 | 0.71 | | 111:20 | 0.59 | 0.51 | 0.67 | UP | 645:65 |
| Hexadecanoic acid | 0.62 | 0.49 | 0.74 | | 111:20 | 0.58 | 0.51 | 0.65 | UP | 645:65 |
| Mat_hdl | 0.52 | 0.38 | 0.65 | | 111:20 | 0.57 | 0.50 | 0.63 | DOWN | 640:65 |
| Linoleic acid | 0.60 | 0.47 | 0.72 | | 111:20 | 0.57 | 0.50 | 0.65 | UP | 645:65 |
| L- Isoleucine | 0.62 | 0.48 | 0.76 | | 111:20 | 0.57 | 0.50 | 0.64 | UP | 645:65 |
| Taurine | 0.65 | 0.52 | 0.79 | UP | 111:20 | 0.53 | 0.46 | 0.60 | | 645:65 |
| L-arginine | 0.64 | 0.52 | 0.77 | UP | 111:20 | 0.52 | 0.45 | 0.60 | | 645:65 |

TABLE 12
Single predictors for total PE in the obese; delineation in accordance to maternal ethnicity (black vs non-black).

| | Prediction of PE in black pregnant obese | | | | | Prediction of PE in non-black obese | | |
|---|---|---|---|---|---|---|---|---|
| | women (stratum:black) | | | | | women stratum:not black) | | |
| | | AUC ICI | AUC uCI | Δ cases: | controls (n): | | AUC ICI | AUC uCI |
| predictors | AUC | 95% CI | 95% CI | controls | cases(n) | AUC | 95% CI | 95% CI |
| Mat_dbp_m | 0.71 | 0.58 | 0.84 | UP | 129:12 | 0.68 | 0.60 | 0.76 |
| Mat_sbp_m | 0.55 | 0.38 | 0.72 | | 129:12 | 0.70 | 0.62 | 0.77 |
| NG-Monomethyl-L-arginine | 0.59 | 0.38 | 0.80 | | 132:12 | 0.66 | 0.58 | 0.74 |
| Biliverdin | 0.64 | 0.45 | 0.82 | | 132:12 | 0.63 | 0.55 | 0.71 |
| Glycyl-glycine* | 0.48 | 0.31 | 0.65 | | 132:12 | 0.65 | 0.56 | 0.73 |
| Dilinoleoyl-glycerol | 0.62 | 0.46 | 0.77 | | 132:12 | 0.61 | 0.53 | 0.69 |
| Etiocholanolone glucuronide | 0.56 | 0.37 | 0.76 | | 132:12 | 0.63 | 0.54 | 0.71 |
| Bilirubin | 0.67 | 0.47 | 0.87 | | 132:12 | 0.60 | 0.51 | 0.69 |
| L-Ieucine | 0.56 | 0.40 | 0.72 | | 132:12 | 0.61 | 0.53 | 0.69 |
| Mat_trig | 0.67 | 0.53 | 0.81 | UP | 132:12 | 0.58 | 0.50 | 0.66 |
| Mat_prev pet | 0.55 | 0.39 | 0.72 | | 97:6 | 0.62 | 0.52 | 0.72 |
| PIGF | 0.61 | 0.42 | 0.81 | | 132:12 | 0.58 | 0.50 | 0.67 |
| 25-Hydroxyvitamin D3 | 0.56 | 0.37 | 0.75 | | 110:10 | 0.61 | 0.52 | 0.69 |
| 1-heptadecanoy1-2-hydroxy-sn- | 0.64 | 0.48 | 0.80 | | 132:12 | 0.58 | 0.50 | 0.66 |
| glycero-3-phosphocholine | | | | | | | | |
| L-(+)-Ergothioneine* | 0.70 | 0.55 | 0.85 | DOWN | 132:12 | 0.56 | 0.48 | 0.64 |
| Octadecenoic acid | 0.56 | 0.38 | 0.74 | | 132:12 | 0.58 | 0.50 | 0.66 |
| Met_XXX | 0.51 | 0.32 | 0.69 | | 132:12 | 0.61 | 0.52 | 0.70 |
| Mat_bmi | 0.56 | 0.34 | 0.78 | | 132:12 | 0.59 | 0.51 | 0.68 |
| Hexadecanoic acid | 0.56 | 0.39 | 0.73 | | 132.12 | 0.58 | 0.50 | 0.66 |
| Mat_hdl | 0.73 | 0.62 | 0.83 | DOWN | 132:12 | 0.53 | 0.46 | 0.61 |
| Linoleic acid | 0.43 | 0.24 | 0.63 | | 132:12 | 0.58 | 0.50 | 0.65 |
| L- Isoleucine | 0.52 | 0.36 | 0.68 | | 132:12 | 0.59 | 0.51 | 0.67 |
| Stearoylcarnitine | 0.69 | 0.54 | 0.84 | DOWN | 132:12 | 0.47 | 0.39 | 0.56 |
| | Prediction of PE | | | | | | | |
| | in non-black obese | | Δ AUC: | | | | | |
| | women stratum:not black) | | black − | Prediction of PE in the obese (stratum:all) | | | | |
| | Δ cases: | controls (n): | Non- | | AUC ICI | AUC uCI | Δ cases: | controls (n): |
| predictors | controls | cases (n) | black | AUC | 95% CI | 95% CI | controls | cases (n) |
| Mat_dbp_m | UP | 504:51 | 0.03 | 0.68 | 0.61 | 0.75 | UP | 633:63 |
| Mat_sbp_m | UP | 504:51 | −0.15 | 0.67 | 0.60 | 0.74 | UP | 633:63 |
| NG-Monomethyl-L-arginine | DOWN | 513:53 | −0.07 | 0.65 | 0.57 | 0.72 | DOWN | 645:65 |
| Biliverdin | DOWN | 511:53 | 0.01 | 0.62 | 0.55 | 0.69 | DOWN | 643:65 |
| Glycyl-glycine* | DOWN | 513:53 | −0.16 | 0.62 | 0.54 | 0.70 | DOWN | 645:65 |
| Dilinoleoyl-glycerol | UP | 513:53 | 0.00 | 0.61 | 0.54 | 0.68 | UP | 645:65 |
| Etiocholanolone glucuronide | UP | 513:53 | −0.06 | 0.61 | 0.54 | 0.69 | UP | 645:65 |
| Bilirubin | DOWN | 513:53 | 0.07 | 0.62 | 0.54 | 0.70 | DOWN | 645:65 |
| L-Ieucine | UP | 513:53 | −0.05 | 0.60 | 0.53 | 0.67 | UP | 645:65 |
| Mat_trig | UP | 508:53 | 0.09 | 0.60 | 0.53 | 0.67 | UP | 640:65 |
| Mat_prev pet | n/a | 265:23 | −0.07 | 0.60 | 0.52 | 0.69 | n/a | 362:29 |
| PIGF | DOWN | 513:53 | 0.03 | 0.60 | 0.52 | 0.67 | DOWN | 645:65 |
| 25-Hydroxyvitamin D3 | DOWN | 458:49 | −0.05 | 0.59 | 0.52 | 0.67 | DOWN | 568:59 |
| 1-heptadecanoy1-2-hydroxy-sn- | DOWN | 513:53 | 0.06 | 0.59 | 0.51 | 0.66 | DOWN | 645:65 |
| glycero-3-phosphocholine | | | | | | | | |
| L-(+)-Ergothioneine* | | 513:53 | 0.14 | 0.58 | 0.51 | 0.66 | DOWN | 645:65 |
| Octadecenoic acid | UP | 513:53 | −0.02 | 0.58 | 0.51 | 0.65 | UP | 645:65 |
| Met_XXX | DOWN | 513:53 | −0.11 | 0.59 | 0.51 | 0.67 | DOWN | 645:65 |
| Mat_bmi | UP | 513:53 | −0.03 | 0.59 | 0.51 | 0.67 | UP | 645:65 |
| Hexadecanoic acid | UP | 513:53 | −0.02 | 0.58 | 0.51 | 0.65 | UP | 645:65 |
| Mat_hdl | | 508:53 | 0.19 | 0.57 | 0.50 | 0.63 | DOWN | 640:65 |
| Linoleic acid | UP | 513:53 | −0.14 | 0.57 | 0.50 | 0.65 | UP | 645:65 |
| L- Isoleucine | UP | 513:53 | −0.07 | 0.57 | 0.50 | 0.64 | UP | 645:65 |
| Stearoylcarnitine | UP | 513:53 | 0.22 | 0.55 | 0.48 | 0.63 | | 645:65 |

TABLE 13
Single predictors for total PE in the obese; delineation in accordance to fetal sex (male vs female).

| | | | | | | Prediction of PE in pregnant | | |
|---|---|---|---|---|---|---|---|---|
| | Prediction of PE in pregnant obese women with | | | | | obese women with female | | |
| | male offspring (stratum:male) | | | | | offspring (stratum:female) | | |
| | | AUC ICI | AUC uCI | Δ cases: | controls (n): | | AUC ICI | AUC uCI |
| predictors | AUC | 95% CI | 95% CI | controls | cases (n) | AUC | 95% CI | 95% CI |
| Mat_dbp_m | 0.80 | 0.74 | 0.87 | UP | 323:25 | 0.60 | 0.50 | 0.70 |
| Mat_sbp_m | 0.72 | 0.62 | 0.82 | UP | 323:25 | 0.63 | 0.53 | 0.73 |
| NG-Monomethyl-L-arginine | 0.63 | 0.50 | 0.76 | DOWN | 328:25 | 0.66 | 0.57 | 0.76 |
| Biliverdin | 0.62 | 0.50 | 0.75 | DOWN | 328:25 | 0.63 | 0.54 | 0.72 |
| Glycyl-glycine | 0.63 | 0.49 | 0.76 | | 328:25 | 0.62 | 0.53 | 0.72 |
| Dilinoleoyl-glycerol | 0.61 | 0.51 | 0.72 | UP | 328:25 | 0.61 | 0.52 | 0.70 |
| Etiocholanolone glucuronide* | 0.66 | 0.54 | 0.78 | UP | 328:25 | 0.58 | 0.48 | 0.68 |
| Bilirubin | 0.57 | 0.43 | 0.70 | | 328:25 | 0.65 | 0.55 | 0.75 |
| L-Ieucine* | 0.52 | 0.42 | 0.63 | | 328:25 | 0.65 | 0.56 | 0.74 |
| Mat_trig | 0.67 | 0.57 | 0.77 | UP | 324:25 | 0.54 | 0.45 | 0.64 |
| Mat_prev pet | 0.59 | 0.43 | 0.75 | | 179:8 | 0.61 | 0.51 | 0.71 |
| PIGF | 0.51 | 0.39 | 0.63 | | 328:25 | 0.65 | 0.55 | 0.74 |
| 25-Hydroxyvitamin D3 | 0.62 | 0.49 | 0.75 | | 294:21 | 0.59 | 0.49 | 0.69 |
| 1-heptadecanoy1-2-hydroxy-sn- | 0.61 | 0.49 | 0.73 | | 328:25 | 0.58 | 0.48 | 0.67 |
| glycero-3-phosphocholine | | | | | | | | |
| L-(+)-Ergothioneine | 0.58 | 0.45 | 0.71 | | 328:25 | 0.58 | 0.49 | 0.67 |
| Octadecenoic acid | 0.59 | 0.49 | 0.70 | | 328:25 | 0.56 | 0.47 | 0.66 |
| Met_XXX | 0.61 | 0.47 | 0.74 | | 328:25 | 0.57 | 0.47 | 0.67 |
| Mat_bmi | 0.59 | 0.46 | 0.71 | | 328:25 | 0.59 | 0.48 | 0.69 |
| Hexadecanoic acid | 0.59 | 0.48 | 0.70 | | 328:25 | 0.57 | 0.47 | 0.67 |
| Mat_hdl | 0.48 | 0.38 | 0.58 | | 324:25 | 0.60 | 0.51 | 0.69 |
| Linoleic acid | 0.60 | 0.49 | 0.71 | | 328:25 | 0.56 | 0.46 | 0.65 |
| L- Isoleucine | 0.51 | 0.40 | 0.62 | | 328:25 | 0.61 | 0.51 | 0.70 |
| | Prediction of PE in pregnant | | Δ AUC: | | | | | |
| | obese women with female | | Male | | | | | |
| | offspring (stratum:female) | | fetus − | Prediction of PE in the obese (stratum:all | | | | |
| | Δ cases: | controls(n): | Female | | AUC ICI | AUC uCI | Δ cases: | controls (n): |
| predictors | controls | cases (n) | fetus | AUC | 95% CI | 95% CI | controls | cases (n) |
| Mat_dbp_m | UP | 308:38 | 0.21 | 0.68 | 0.61 | 0.75 | UP | 633:63 |
| Mat_sbp_m | UP | 308:38 | 0.09 | 0.67 | 0.60 | 0.74 | UP | 633:63 |
| NG-Monomethyl-L-arginine | DOWN | 315:40 | −0.03 | 0.65 | 0.57 | 0.72 | DOWN | 645:65 |
| Biliverdin | DOWN | 313:40 | −0.01 | 0.62 | 0.55 | 0.69 | DOWN | 643:65 |
| Glycyl-glycine | DOWN | 315:40 | 0.01 | 0.62 | 0.54 | 0.70 | DOWN | 645:65 |
| Dilinoleoyl-glycerol | UP | 315:40 | 0.00 | 0.61 | 0.54 | 0.68 | UP | 645:65 |
| Etiocholanolone glucuronide* | | 315:40 | 0.07 | 0.61 | 0.54 | 0.69 | UP | 645:65 |
| Bilirubin | DOWN | 315:40 | −0.08 | 0.62 | 0.54 | 0.70 | DOWN | 645:65 |
| L-Ieucine* | UP | 315:40 | −0.13 | 0.60 | 0.53 | 0.67 | UP | 645:65 |
| Mat_trig | UP | 314:40 | 0.12 | 0.60 | 0.53 | 0.67 | UP | 640:65 |
| Mat_prev pet | n/a | 181:21 | −0.02 | 0.60 | 0.52 | 0.69 | n/a | 362:29 |
| PIGF | DOWN | 315:40 | −0.13 | 0.60 | 0.52 | 0.67 | DOWN | 645:65 |
| 25-Hydroxyvitamin D3 | | 272:38 | 0.03 | 0.59 | 0.52 | 0.67 | DOWN | 568:59 |
| 1-heptadecanoy1-2-hydroxy-sn- | | 315:40 | 0.03 | 0.59 | 0.51 | 0.66 | DOWN | 645:65 |
| glycero-3-phosphocholine | | | | | | | | |
| L-(+)-Ergothioneine | | 315:40 | 0.00 | 0.58 | 0.51 | 0.66 | DOWN | 645:65 |
| Octadecenoic acid | | 315:40 | 0.03 | 0.58 | 0.51 | 0.65 | UP | 645:65 |
| Met_XXX | | 315:40 | 0.03 | 0.59 | 0.51 | 0.67 | DOWN | 645:65 |
| Mat_bmi | | 315:40 | 0.00 | 0.59 | 0.51 | 0.67 | UP | 645:65 |
| Hexadecanoic acid | | 315:40 | 0.02 | 0.58 | 0.51 | 0.65 | UP | 645:65 |
| Mat_hdl | DOWN | 314:40 | −0.12 | 0.57 | 0.50 | 0.63 | DOWN | 640:65 |
| Linoleic acid | | 315:40 | 0.05 | 0.57 | 0.50 | 0.65 | UP | 645:65 |
| L- Isoleucine | UP | 315:40 | −0.09 | 0.57 | 0.50 | 0.64 | UP | 645:65 |

TABLE 14
Single predictors for PE in the obese; delineation in accordance to the sub-outcomes preterm PE and term PE.

| | | | | | | Prediction of term PE | | |
|---|---|---|---|---|---|---|---|---|
| | Prediction of preterm PE in pregnant obese | | | | | in pregnant obese women | | |
| | women (stratum:preterm PE) | | | | | (stratum:term PE) | | |
| | | AUC ICI | AUC uCI | Δ cases: | controls(n): | | AUC ICI | AUC uCI |
| Predictors | AUC | 95% CI | 95% CI | controls | cases (n) | AUC | 95% CI | 95% CI |
| Mat_d bp_m | 0.71 | 0.60 | 0.83 | UP | 633:13 | 0.67 | 0.59 | 0.75 |
| Mat_sbp_m | 0.69 | 0.55 | 0.83 | UP | 633:13 | 0.66 | 0.58 | 0.74 |
| NG-Monomethyl-L-arginine | 0.62 | 0.44 | 0.81 | | 645:13 | 0.66 | 0.57 | 0.74 |
| Biliverdin | 0.57 | 0.39 | 0.76 | | 643:13 | 0.64 | 0.56 | 0.71 |
| Glycyl-glycine | 0.59 | 0.42 | 0.75 | | 645:13 | 0.63 | 0.54 | 0.72 |
| Dilinoleoyl-glycerol | 0.57 | 0.42 | 0.73 | | 645:13 | 0.62 | 0.55 | 0.70 |
| Etiocholanolone glucuronide | 0.62 | 0.47 | 0.78 | | 645:13 | 0.61 | 0.53 | 0.70 |
| Bilirubin | 0.56 | 0.35 | 0.76 | | 645:13 | 0.63 | 0.55 | 0.72 |
| L-Ieucine | 0.64 | 0.50 | 0.78 | UP | 645:13 | 0.59 | 0.51 | 0.67 |
| Mat_trig | 0.61 | 0.45 | 0.78 | | 640:13 | 0.59 | 0.52 | 0.67 |
| Mat_prev pet | 0.72 | 0.50 | 0.94 | n/a | 362:6 | 0.58 | 0.49 | 0.66 |
| PIGF | 0.76 | 0.63 | 0.90 | DOWN | 645:13 | 0.55 | 0.47 | 0.64 |
| 25-Hydroxyvitamin D3 | 0.55 | 0.36 | 0.75 | | 568:11 | 0.60 | 0.52 | 0.69 |
| 1-heptadecanoy1-2-hydroxy-sn- | 0.67 | 0.54 | 0.80 | DOWN | 645:13 | 0.57 | 0.48 | 0.65 |
| glycero-3-phosphocholine* | | | | | | | | |
| L-(+)-Ergothioneine | 0.64 | 0.48 | 0.80 | | 645:13 | 0.57 | 0.49 | 0.65 |
| Octadecenoic acid | 0.55 | 0.41 | 0.69 | | 645:13 | 0.59 | 0.50 | 0.67 |
| Met_XXX | 0.58 | 0.42 | 0.73 | | 645:13 | 0.59 | 0.50 | 0.68 |
| Mat_bmi | 0.52 | 0.38 | 0.67 | | 645:13 | 0.61 | 0.52 | 0.70 |
| Hexadecanoic acid | 0.54 | 0.39 | 0.68 | | 645:13 | 0.59 | 0.51 | 0.67 |
| Mat_hdl | 0.62 | 0.47 | 0.77 | | 640:13 | 0.55 | 0.48 | 0.63 |
| Linoleic acid* | 0.47 | 0.32 | 0.62 | | 645:13 | 0.58 | 0.50 | 0.67 |
| L-Isoleucine | 0.63 | 0.50 | 0.76 | UP | 645:13 | 0.56 | 0.47 | 0.64 |
| Urea | 0.61 | 0.45 | 0.77 | | 645:13 | 0.59 | 0.51 | 0.67 |
| | Prediction of term PE | | | | | | | |
| | in pregnant obese women | | Δ AUC: | | | | | |
| | (stratum:term PE) | | Preterm | Prediction of PE in the obese (stratum:all) | | | | |
| | Δ cases: | controls(n): | PE − | | AUC ICI | AUC uCI | Δ cases: | controls (n): |
| Predictors | controls | cases (n) | Term PE | AUC | 95% CI | 95% CI | controls | cases (n) |
| Mat_d bp_m | UP | 633:50 | 0.04 | 0.68 | 0.61 | 0.75 | UP | 633:63 |
| Mat_sbp_m | UP | 633:50 | 0.03 | 0.67 | 0.60 | 0.74 | UP | 633:63 |
| NG-Monomethyl-L-arginine | DOWN | 645:52 | −0.03 | 0.65 | 0.57 | 0.72 | DOWN | 645:65 |
| Biliverdin | DOWN | 643:52 | −0.06 | 0.62 | 0.55 | 0.69 | DOWN | 643:65 |
| Glycyl-glycine | DOWN | 645:52 | −0.04 | 0.62 | 0.54 | 0.70 | DOWN | 645:65 |
| Dilinoleoyl-glycerol | UP | 645:52 | −0.05 | 0.61 | 0.54 | 0.68 | UP | 645:65 |
| Etiocholanolone glucuronide | UP | 645:52 | 0.01 | 0.61 | 0.54 | 0.69 | UP | 645:65 |
| Bilirubin | DOWN | 645:52 | −0.08 | 0.62 | 0.54 | 0.70 | DOWN | 645:65 |
| L-Ieucine | UP | 645:52 | 0.05 | 0.60 | 0.53 | 0.67 | UP | 645:65 |
| Mat_trig | UP | 640:52 | 0.02 | 0.60 | 0.53 | 0.67 | UP | 640:65 |
| Mat_prev pet | | 362:23 | 0.14 | 0.60 | 0.52 | 0.69 | n/a | 362:29 |
| PIGF | | 645:52 | 0.21 | 0.60 | 0.52 | 0.67 | DOWN | 645:65 |
| 25-Hydroxyvitamin D3 | DOWN | 568:48 | −0.05 | 0.59 | 0.52 | 0.67 | DOWN | 568:59 |
| 1-heptadecanoy1-2-hydroxy-sn- | | 645:52 | 0.10 | 0.59 | 0.51 | 0.66 | DOWN | 645:65 |
| glycero-3-phosphocholine* | | | | | | | | |
| L-(+)-Ergothioneine | | 645:52 | 0.07 | 0.58 | 0.51 | 0.66 | DOWN | 645:65 |
| Octadecenoic acid | UP | 645:52 | −0.03 | 0.58 | 0.51 | 0.65 | UP | 645:65 |
| Met_XXX | DOWN | 645:52 | −0.01 | 0.59 | 0.51 | 0.67 | DOWN | 645:65 |
| Mat_bmi | UP | 645:52 | −0.09 | 0.59 | 0.51 | 0.67 | UP | 645:65 |
| Hexadecanoic acid | UP | 645:52 | −0.05 | 0.58 | 0.51 | 0.65 | UP | 645:65 |
| Mat_hdl | | 640:52 | 0.06 | 0.57 | 0.50 | 0.63 | DOWN | 640:65 |
| Linoleic acid* | UP | 645:52 | −0.11 | 0.57 | 0.50 | 0.65 | UP | 645:65 |
| L-Isoleucine | | 645:52 | 0.07 | 0.57 | 0.50 | 0.64 | UP | 645:65 |
| Urea | UP | 645:52 | 0.02 | 0.55 | 0.47 | 0.63 | | 645:65 |

TABLE 15
Predictors relevant to prognostic multivariable models for the prediction of PE in the obese pregnancy population.

| | | Number | Univariable | | | | | | | | | | | | |
|---|---|---|---|---|---|---|---|---|---|---|---|---|---|---|---|
| | Predictor type | of Models | performance | A | B | C | D | E | F | G | H | I | J | K | L |
| n models | | | | 381 | 169 | 6 | 49 | 23 | 25 | 3 | 260 | 17 | 99 | 0 | 192 |
| n predictors in models | | | | 2 to 4 | 2 to 4 | 2 to 4 | 2 to 4 | 2 to 4 | 2 to 4 | 2 to | 2 to 4 | 2 to | 2 to 4 | 2 to 4 | 2 to |
| | | | | | | | | | | 4 | | 4 | | | 4 |
| Predictors | | | | | | | | | | | | | | | |
| Biliverdin | metabolite | 448 | TRUE | 131 | 70 | 1 | 9 | 3 | 13 | | 100 | 13 | 39 | | 69 |
| Met_XXX | metabolite | 394 | TRUE | 126 | 54 | | 10 | | | | 124 | 9 | 10 | | 61 |
| Glycyl-glycine | metabolite | 278 | TRUE | 103 | 23 | 2 | 2 | 6 | 7 | | 59 | 1 | 31 | | 44 |
| NG-Monomethyl-L-arginine | metabolite | 250 | TRUE | 53 | 39 | | 14 | 2 | | | 65 | | 21 | | 56 |
| Etiocholanolone glucuronide | metabolite | 246 | TRUE | 91 | 30 | 1 | 12 | 7 | 4 | | 49 | 1 | 14 | | 37 |
| Eicosapentaenoic acid | metabolite | 195 | FALSE | 58 | 18 | | 7 | 5 | 2 | | 47 | 1 | 17 | | 40 |
| Dilinoleoyl-glycerol | metabolite | 157 | TRUE | 55 | 24 | | 13 | | | | 30 | | 9 | | 26 |
| L-leucine | metabolite | 145 | TRUE | 50 | 32 | | 2 | 2 | 1 | | 29 | | 16 | | 13 |
| Hexadecanoic acid | metabolite | 139 | TRUE | 40 | 19 | 1 | 2 | 3 | 4 | | 36 | 1 | 10 | | 23 |
| 25-Hydroxyvitamin D3 | metabolite | 121 | TRUE | 44 | 3 | 1 | 1 | | 4 | | 36 | 2 | 2 | | 28 |
| PIGF | other | 106 | TRUE | 39 | 40 | | | 1 | | | 14 | | 12 | | |
| Mat_sbp_m | other | 103 | TRUE | 33 | 13 | 1 | 2 | 2 | 6 | | 18 | 2 | 7 | | 19 |
| Mat_hdl | other | 95 | TRUE | 51 | 9 | | 7 | | | 1 | | | 25 | | 2 |
| Linoleic acid | metabolite | 93 | TRUE | 29 | 13 | | 5 | | 1 | | 22 | 2 | 5 | | 16 |
| Octadecenoic acid | metabolite | 91 | TRUE | 29 | 6 | | 3 | | 3 | 1 | 28 | 1 | 2 | | 18 |
| Mat_age_LT25 | other | 87 | FALSE | 36 | | | 11 | | | | 15 | | 8 | | 17 |
| Stearoylcarnitine | metabolite | 75 | TRUE | 50 | 3 | | 2 | 5 | | 1 | 2 | 1 | 8 | | 3 |
| Stearic acid | metabolite | 70 | FALSE | 22 | 18 | 2 | 6 | | 1 | 1 | 10 | 2 | 2 | | 6 |
| 8,11,14 Eicosatrienoic acid | metabolite | 65 | TRUE | 18 | 4 | 1 | | 3 | 2 | | 19 | 1 | 2 | | 15 |
| 1-Palmitoy1-2-hydroxy-sn- | metabolite | 58 | FALSE | 19 | 11 | | 6 | | | | 7 | 3 | | | 12 |
| glycero-3-phosphocholine | | | | | | | | | | | | | | | |
| L- Isoleucine | metabolite | 56 | TRUE | 19 | 8 | | 4 | 1 | | | 14 | | 7 | | 3 |
| Bilirubin | metabolite | 50 | TRUE | 15 | 10 | | | 2 | 1 | | 3 | | 2 | | 17 |
| PIGF MoM | other | 41 | TRUE | 13 | 13 | | 1 | | | | 2 | | 12 | | |
| Mat_dbp_m | other | 41 | TRUE | 11 | 5 | | 1 | | 2 | | 10 | | 2 | | 10 |
| L-arginine | metabolite | 41 | TRUE | 5 | 4 | | 1 | 2 | 5 | | 7 | | 11 | | 6 |
| L-(+)-Ergothioneine | metabolite | 34 | TRUE | 21 | 1 | | 1 | 1 | | | 2 | | 5 | | 3 |
| Myristic acid | metabolite | 32 | FALSE | 11 | 3 | | | | 2 | | 10 | 1 | | | 5 |
| L-Palmitoylcarnitine | metabolite | 31 | FALSE | 8 | 13 | | 5 | | | | 3 | | 2 | | |
| Arachidonic acid | metabolite | 29 | FALSE | 13 | 1 | | | 1 | 2 | | 6 | 1 | 1 | | 4 |
| Mat_trig | other | 24 | TRUE | 8 | 12 | | 1 | | | | | | | | 3 |
| Urea | metabolite | 23 | TRUE | 1 | 5 | | 1 | | | | | | 1 | | 15 |
| Mat_age_GE35 | other | 19 | FALSE | 2 | | | | | 1 | | 16 | | | | |
| Choline | metabolite | 19 | FALSE | | 9 | | | | | | 5 | | | | 5 |
| Taurine | metabolite | 17 | TRUE | 6 | 5 | 2 | | | 3 | 1 | | | | | |
| Mat_bmi | other | 14 | TRUE | 7 | 1 | | 1 | | | | 2 | | | | 3 |
| Docosahexaenoic acid | metabolite | 14 | FALSE | 4 | | | 2 | | 1 | | 5 | 1 | | | 1 |
| Asymmetric dimethylarginine | metabolite | 14 | TRUE | 3 | 1 | 1 | | 2 | | | 2 | | 2 | | 3 |
| L-methionine | metabolite | 10 | FALSE | 1 | | | | 1 | | | 2 | | 2 | | 4 |
| 3-Hydroxputanoic acid | metabolite | 9 | FALSE | | | | | 3 | 1 | 2 | 3 | | | | |
| 2-Hydroxputanoic acid | metabolite | 8 | FALSE | | 1 | | | | 1 | | 5 | | 1 | | |
| 1 -heptadecanoy1-2-hydroxy- | metabolite | 7 | TRUE | 5 | 2 | | | | | | | | | | |
| sn-glycero-3-phosphocholine | | | | | | | | | | | | | | | |
| Decanoylcarnitine | metabolite | 7 | FALSE | 1 | | | 3 | | | | 3 | | | | |
| L-Acetylcarnitine | metabolite | 5 | FALSE | | 2 | | | | | | 3 | | | | |
| Citrulline | metabolite | 4 | FALSE | 1 | | 1 | | | 2 | | | | | | |
| Mat_prev_pet | other | 3 | TRUE | 1 | | 2 | | | | | | | | | |
| Dodecanoyl-1-carnitine | metabolite | 3 | FALSE | | | | | 1 | 1 | | 1 | | | | |
| uncomp_pregn_score | other | 3 | FALSE | | | | | | | | | | 3 | | |
| Mat_Idl_hdl | other | 2 | FALSE | 2 | | | | | | | | | | | |
| Sphingosine 1-phosphate | metabolite | 2 | FALSE | | 2 | | | | | | | | | | |
| gaw_1st_visit | other | 1 | FALSE | | | | | 1 | | | | | | | |
| L-alanine | metabolite | 1 | TRUE | | | | 1 | | | | | | | | |

TABLE 16
Exemplary Multivariable prediction for PE in the obese; stratum: all; outcome: Total PE

| | | all.AUC | | | mean.AUC | | |
|---|---|---|---|---|---|---|---|
| | number of | | AUC ICI | AUC uCI | | AUC ICI | AUC uCI |
| predictors in model | predictors | AUC | 95% CI | 95% CI | AUC | 95% CI | 95% CI |
| 8,11,14 Eicosatrienoic acid, Glycyl-glycine, Stearoylcarnitine, Mat_prev pet | 4 | 0.79 | 0.71 | 0.87 | 0.79 | 0.66 | 0.92 |
| Glycyl-glycine, Biliverdin, Etiocholanolone glucuronide, Mat_sbp_m* | 4 | 0.78 | 0.71 | 0.84 | 0.77 | 0.66 | 0.88 |
| Dilinoleoyl-glycerol, Glycyl-glycine, Biliverdin, Mat_sbp_m | 4 | 0.77 | 0.71 | 0.83 | 0.76 | 0.65 | 0.87 |
| Dilinoleoyl-glycerol, NG-Monomethyl-L-arginine, Biliverdin, Mat_sbp_m | 4 | 0.77 | 0.71 | 0.83 | 0.75 | 0.64 | 0.86 |
| Dilinoleoyl-glycerol, Biliverdin, Etiocholanolone glucuronide, Mat_sbp_m | 4 | 0.77 | 0.70 | 0.83 | 0.75 | 0.64 | 0.87 |
| Biliverdin, Etiocholanolone glucuronide, Met_XXX, Mat_sbp_m | 4 | 0.76 | 0.69 | 0.83 | 0.75 | 0.63 | 0.88 |
| Glycyl-glycine, Biliverdin, Etiocholanolone glucuronide, PIGF | 4 | 0.76 | 0.70 | 0.82 | 0.75 | 0.64 | 0.87 |
| Glycyl-glycine, Biliverdin, Etiocholanolone glucuronide, Mat_hdl | 4 | 0.76 | 0.69 | 0.82 | 0.75 | 0.64 | 0.86 |
| Eicosapentaenoic acid, Biliverdin, Etiocholanolone glucuronide, | 4 | 0.75 | 0.69 | 0.82 | 0.75 | 0.64 | 0.86 |
| Mat_age_LT25 | | | | | | | |
| Glycyl-glycine, Biliverdin, Mat_sbp_m | 3 | 0.75 | 0.69 | 0.82 | 0.75 | 0.63 | 0.87 |

TABLE 17
Exemplary Multivariable prediction for PE in the obese; stratum: no GDM; outcome: Total PE

| | | all.AUC | | | mean.AUC | | |
|---|---|---|---|---|---|---|---|
| | number of | | AUC ICI | AUC uCI | | AUC ICI | AUC uCI |
| predictors in model | predictors | AUC | 95% CI | 95% CI | AUC | 95% CI | 95% CI |
| L-Ieucine, Dilinoleoyl-glycerol, NG-Monomethyl-L-arginine, Biliverdin* | 4 | 0.81 | 0.75 | 0.87 | 0.79 | 0.68 | 0.89 |
| NG-Monomethyl-L-arginine, Biliverdin, Etiocholanolone glucuronide, PIGF | 4 | 0.81 | 0.75 | 0.87 | 0.79 | 0.68 | 0.90 |
| L- Isoleucine, NG-Monomethyl-L-arginine, Mat_dbp_m | 3 | 0.81 | 0.74 | 0.87 | 0.80 | 0.70 | 0.89 |
| NG-Monomethyl-L-arginine, Biliverdin, Etiocholanolone glucuronide, | 4 | 0.80 | 0.74 | 0.87 | 0.77 | 0.65 | 0.90 |
| Mat_hdl | | | | | | | |
| NG-Monomethyl-L-arginine, Biliverdin, Stearic acid, Met_XXX | 4 | 0.80 | 0.73 | 0.87 | 0.80 | 0.67 | 0.92 |
| Hexadecanoic acid, NG-Monomethyl-L-arginine, Biliverdin, Met_XXX | 4 | 0.80 | 0.73 | 0.87 | 0.80 | 0.68 | 0.92 |
| Hexadecanoic acid, NG-Monomethyl-L-arginine, Biliverdin, PIGF | 4 | 0.80 | 0.73 | 0.86 | 0.77 | 0.67 | 0.87 |
| 8,11,14 Eicosatrienoic acid, NG-Monomethyl-L-arginine, Biliverdin, | 4 | 0.79 | 0.72 | 0.86 | 0.78 | 0.66 | 0.90 |
| Met_XXX | | | | | | | |
| Dilinoleoyl-glycerol, Biliverdin, Etiocholanolone glucuronide, PIGF | 4 | 0.79 | 0.72 | 0.86 | 0.77 | 0.65 | 0.90 |
| NG-Monomethyl-L-arginine, Octadecenoic acid, Biliverdin, Met_XXX | 4 | 0.79 | 0.72 | 0.86 | 0.75 | 0.63 | 0.88 |
| Dilinoleoyl-glycerol, NG-Monomethyl-L-arginine, Urea | 3 | 0.79 | 0.72 | 0.86 | 0.76 | 0.64 | 0.88 |
| Hexadecanoic acid, Biliverdin, Met_XXX, PIGF | 4 | 0.79 | 0.72 | 0.86 | 0.77 | 0.65 | 0.88 |
| L-arginine, Glycyl-glycine, NG-Monomethyl-L-arginine, Biliverdin | 4 | 0.78 | 0.71 | 0.85 | 0.76 | 0.62 | 0.90 |
| Eicosapentaenoic acid, Biliverdin, Etiocholanolone glucuronide, PIGF | 4 | 0.78 | 0.71 | 0.86 | 0.78 | 0.66 | 0.90 |
| L-arginine, NG-Monomethyl-L-arginine, Biliverdin, Stearic acid | 4 | 0.78 | 0.72 | 0.85 | 0.77 | 0.65 | 0.88 |
| Linoleic acid, NG-Monomethyl-L-arginine, Eicosapentaenoic acid, Biliverdin | 4 | 0.78 | 0.72 | 0.84 | 0.76 | 0.65 | 0.86 |
| NG-Monomethyl-L-arginine, Biliverdin, Myristic acid, Met_XXX | 4 | 0.78 | 0.70 | 0.86 | 0.76 | 0.62 | 0.89 |
| Hexadecanoic acid, NG-Monomethyl-L-arginine, Eicosapentaenoic acid, | 4 | | | | | | |
| Met_XXX | | 0.78 | 0.71 | 0.85 | 0.76 | 0.65 | 0.88 |
| Choline, NG-Monomethyl-L-arginine, Stearic acid, Met_XXX | 4 | 0.78 | 0.70 | 0.86 | 0.78 | 0.66 | 0.89 |
| Choline, NG-Monomethyl-L-arginine, Octadecenoic acid, | 4 | 0.78 | 0.71 | 0.85 | 0.76 | 0.65 | 0.88 |
| L-Palmitoylcarnitine | | | | | | | |
| Choline, Linoleic acid, NG-Monomethyl-L-arginine, L-Palmitoylcarnitine | 4 | 0.77 | 0.70 | 0.85 | 0.76 | 0.64 | 0.88 |
| L-leucine, Hexadecanoic acid, Biliverdin, Met_XXX | 4 | 0.77 | 0.70 | 0.85 | 0.77 | 0.63 | 0.91 |
| Glycyl-glycine, Biliverdin, Mat_dbp_m | 3 | 0.77 | 0.69 | 0.85 | 0.77 | 0.62 | 0.91 |
| L-leucine, Biliverdin, Stearic acid, Met_XXX | 4 | 0.77 | 0.70 | 0.85 | 0.78 | 0.65 | 0.91 |
| Choline, NG-Monomethyl-L-arginine, Stearoylcarnitine, 1-Palmitoy1-2- | 4 | 0.77 | 0.70 | 0.85 | 0.76 | 0.65 | 0.88 |
| hydroxy-sn-glycero-3-phosphocholine | | | | | | | |
| NG-Monomethyl-L-arginine, Eicosapentaenoic acid, Etiocholanolone | 4 | 0.77 | 0.70 | 0.84 | 0.76 | 0.63 | 0.89 |
| glucuronide, PIGF | | | | | | | |
| Choline, NG-Monomethyl-L-arginine, 1-heptadecanoy1-2-hydroxy-sn- | 4 | 0.77 | 0.70 | 0.84 | 0.76 | 0.65 | 0.87 |
| glycero-3-phosphocholine, 1-Palmitoy1-2-hydroxy-sn-glycero-3- | | | | | | | |
| Biliverdin, Mat_dbp_m , PIGF | 3 | 0.77 | 0.70 | 0.84 | 0.76 | 0.64 | 0.87 |
| Choline, NG-Monomethyl-L-arginine, Mat_trig | 3 | 0.77 | 0.69 | 0.84 | 0.76 | 0.64 | 0.88 |
| L-arginine, Dilinoleoyl-glycerol, NG-Mononnethyl-L-arginine | 3 | 0.77 | 0.69 | 0.84 | 0.75 | 0.62 | 0.88 |
| Linoleic acid, Biliverdin, Met_XXX, PIGF | 4 | 0.77 | 0.69 | 0.84 | 0.77 | 0.65 | 0.89 |
| L-Ieucine, 8,11,14 Eicosatrienoic acid, Biliverdin, Met_XXX | 4 | 0.77 | 0.69 | 0.85 | 0.75 | 0.61 | 0.89 |
| Choline, NG-Mononnethyl-L-arginine, L-Palnnitoylcarnitine, 1-PaInnitoy1-2- | 4 | 0.77 | 0.70 | 0.84 | 0.76 | 0.66 | 0.87 |
| hydroxy-sn-glycero-3-phosphocholine | | | | | | | |
| L-arginine, Choline, NG-Mononnethyl-L-arginine | 3 | 0.77 | 0.70 | 0.84 | 0.76 | 0.65 | 0.88 |
| Hexadecanoic acid, NG-Mononnethyl-L-arginine, Biliverdin | 3 | 0.77 | 0.70 | 0.83 | 0.76 | 0.64 | 0.87 |
| Biliverdin, Etiocholanolone glucuronide, PIGF | 3 | 0.77 | 0.69 | 0.84 | 0.75 | 0.62 | 0.88 |
| Biliverdin, Stearic acid, Met_XXX, Mat_hdl | 4 | 0.77 | 0.69 | 0.85 | 0.77 | 0.63 | 0.91 |
| Dilinoleoyl-glycerol, Biliverdin, Stearic acid, Met_XXX | 4 | 0.76 | 0.68 | 0.84 | 0.77 | 0.63 | 0.91 |
| Hexadecanoic acid, NG-Mononnethyl-L-arginine, Met_XXX, PIGF | 4 | 0.76 | 0.69 | 0.84 | 0.75 | 0.63 | 0.87 |
| NG-Mononnethyl-L-arginine, Mat_sbp_nn | 2 | 0.76 | 0.69 | 0.84 | 0.78 | 0.69 | 0.87 |
| Octadecenoic acid, Biliverdin, Met_XXX, PIGF | 4 | 0.76 | 0.69 | 0.84 | 0.76 | 0.65 | 0.88 |
| NG-Mononnethyl-L-arginine, Mat_dbp_nn | 2 | 0.76 | 0.68 | 0.84 | 0.76 | 0.64 | 0.87 |
| Dilinoleoyl-glycerol, Biliverdin, Myristic acid, Met_XXX | 4 | 0.76 | 0.68 | 0.84 | 0.75 | 0.62 | 0.89 |
| NG-Mononnethyl-L-arginine, Asymmetric dinnethylarginine | 2 | 0.76 | 0.68 | 0.83 | 0.76 | 0.65 | 0.87 |
| NG-Mononnethyl-L-arginine, Urea, Mat_trig | 3 | 0.76 | 0.69 | 0.82 | 0.75 | 0.64 | 0.86 |
| L-Ieucine, NG-Mononnethyl-L-arginine | 2 | 0.75 | 0.67 | 0.83 | 0.75 | 0.64 | 0.87 |
| NG-Mononnethyl-L-arginine, Stearic acid, Met_XXX | 3 | 0.75 | 0.67 | 0.83 | 0.75 | 0.63 | 0.87 |

TABLE 18
Exemplary Multivariable prediction for PE in the obese; stratum: GDM; outcome: Total PE

| | | all.AUC | | | mean.AUC | | |
|---|---|---|---|---|---|---|---|
| | number of | | AUC ICI | AUC uCI | | AUC ICI | AUC uCI |
| predictors in model | predictors | AUC | 95% CI | 95% CI | AUC | 95% CI | 95% CI |
| Hexadecanoic acid, Stearic acid, Mat_prev_pet | 3 | 0.88 | 0.76 | 0.99 | 0.83 | 0.57 | 1.00 |
| 25-Hydroxyvitamin D3, Citrulline, Biliverdin, | 4 | 0.83 | 0.72 | 0.94 | 0.78 | 0.53 | 1.00 |
| Etiocholanolone glucuronide* | | | | | | | |
| Taurine, Mat_sbp_m | 2 | 0.83 | 0.73 | 0.92 | 0.81 | 0.64 | 0.98 |
| 8,11,14 Eicosatrienoic acid, Stearic acid, | 3 | 0.81 | 0.62 | 1.00 | 0.84 | 0.64 | 1.00 |
| Mat_prev_pet | | | | | | | |
| Glycyl-glycine, Asymmetric dimethylarginine | 2 | 0.77 | 0.67 | 0.87 | 0.77 | 0.58 | 0.96 |

TABLE 19
Exemplary Multivariable prediction for PE in the obese; stratum: 1st time pregnant; outcome: Total PE

| | | all · AUC | | | mean · AUC | | |
|---|---|---|---|---|---|---|---|
| | number of | | AUC lCI | AUC uCI | | AUC lCI | AUC uCI |
| predictors in model | predictors | AUC | 95% CI | 95% CI | AUC | 95% CI | 95% CI |
| Eicosapentaenoic acid, Biliverdin, Etiocholanolone | 4 | 0.77 | 0.69 | 0.85 | 0.77 | 0.63 | 0.90 |
| glucuronide, Mat_age_LT25 | | | | | | | |
| Biliverdin, Etiocholanolone glucuronide, | 4 | 0.77 | 0.69 | 0.85 | 0.77 | 0.62 | 0.91 |
| L-(+)-Ergothioneine, Mat_age_LT25* | | | | | | | |

TABLE 20
Exemplary Multivariable prediction for PE in the obese; stratum: 2nd or more time pregnant; outcome: Total PE

| | | all · AUC | | | mean · AUC | | |
|---|---|---|---|---|---|---|---|
| | number of | | AUC lCI | AUC uCI | | AUC lCI | AUC uCI |
| predictors in model | predictors | AUC | 95% CI | 95% CI | AUC | 95% CI | 95% CI |
| L-arginine, Glycyl-glycine, NG-Monomethyl-L- | 4 | 0.78 | 0.68 | 0.89 | 0.75 | 0.54 | 0.95 |
| arginine, | | | | | | | |
| Asymmetric dimethylarginine* | | | | | | | |
| L-(+)-Ergothioneine, Mat_sbp_m | 2 | 0.77 | 0.68 | 0.86 | 0.76 | 0.59 | 0.93 |
| 8, 11, 14 Eicosatrienoic acid, Eicosapentaenoic acid, | 4 | 0.77 | 0.66 | 0.87 | 0.76 | 0.55 | 0.97 |
| Biliverdin, Etiocholanolone glucuronide | | | | | | | |
| Glycyl-glycine, Mat_sbp_m | 2 | 0.76 | 0.67 | 0.86 | 0.77 | 0.58 | 0.94 |

TABLE 21
Exemplary Multivariable prediction for PE in the obese; stratum: bmi > 40; outcome: Total PE

| | | all · AUC | | | mean · AUC | | |
|---|---|---|---|---|---|---|---|
| | number of | | AUC lCI | AUC uCI | | AUC lCI | UC uCI |
| predictors in model | predictors | AUC | 95% CI | 95% CI | AUC | 95% CI | 95% CI |
| Biliverdin, Myristic acid, Mat_sbp_m* | 3 | 0.86 | 0.77 | 0.95 | 0.85 | 0.70 | 0.99 |
| Hexadecanoic acid, Biliverdin, Mat_sbp_m | 3 | 0.86 | 0.77 | 0.95 | 0.85 | 0.70 | 0.99 |
| 8, 11, 14 Eicosatrienoic acid, Biliverdin, Mat_sbp_m | 3 | 0.86 | 0.77 | 0.94 | 0.85 | 0.70 | 0.98 |
| Arachidonic acid, Biliverdin, Mat_sbp_m | 3 | 0.85 | 0.76 | 0.93 | 0.85 | 0.69 | 0.99 |
| Dodecanoyl-l-carnitine, Biliverdin, Mat_sbp_m | 3 | 0.84 | 0.76 | 0.92 | 0.85 | 0.72 | 0.98 |
| Linoleic acid, Biliverdin, Mat_sbp_m | 3 | 0.84 | 0.73 | 0.94 | 0.83 | 0.67 | 0.99 |
| 8, 11, 14 Eicosatrienoic acid, Biliverdin, Mat_dbp_m | 3 | 0.83 | 0.75 | 0.92 | 0.83 | 0.69 | 0.97 |
| Biliverdin, Myristic acid, Mat_age_GE35 | 3 | 0.83 | 0.74 | 0.91 | 0.83 | 0.69 | 0.97 |
| Arachidonic acid, Biliverdin, Mat_dbp_m | 3 | 0.82 | 0.73 | 0.92 | 0.81 | 0.64 | 0.97 |
| Biliverdin, Etiocholanolone glucuronide | 2 | 0.81 | 0.71 | 0.92 | 0.82 | 0.65 | 0.97 |
| Taurine, Eicosapentaenoic acid, Etiocholanolone glucuronide | 3 | 0.80 | 0.69 | 0.92 | 0.82 | 0.66 | 0.97 |
| 25-Hydroxyvitamin D3, L-arginine, Glycyl-glycine | 3 | 0.80 | 0.69 | 0.91 | 0.80 | 0.61 | 0.99 |
| Docosahexaenoic acid, Taurine, Etiocholanolone glucuronide | 3 | 0.80 | 0.68 | 0.92 | 0.81 | 0.63 | 0.97 |
| Glycyl-glycine, Bilirubin | 2 | 0.80 | 0.67 | 0.92 | 0.79 | 0.61 | 0.96 |
| Hexadecanoic acid, Biliverdin | 2 | 0.80 | 0.69 | 0.90 | 0.79 | 0.61 | 0.97 |
| Octadecenoic acid, Biliverdin | 2 | 0.79 | 0.69 | 0.90 | 0.79 | 0.60 | 0.98 |
| Glycyl-glycine, Biliverdin | 2 | 0.79 | 0.68 | 0.90 | 0.78 | 0.58 | 0.97 |
| 2-Hydroxbutanoic acid, L-leucine, Glycyl-glycine, Stearic acid | 4 | 0.79 | 0.70 | 0.87 | 0.75 | 0.59 | 0.91 |
| L-arginine, Hexadecanoic acid, Eicosapentaenoic acid | 3 | 0.78 | 0.68 | 0.87 | 0.76 | 0.57 | 0.96 |
| L-arginine, Glycyl-glycine, Taurine | 3 | 0.77 | 0.67 | 0.88 | 0.76 | 0.55 | 0.97 |
| 25-Hydroxyvitamin D3, 3-Hydroxybutanoic acid, | 4 | 0.77 | 0.67 | 0.87 | 0.76 | 0.57 | 0.95 |
| Glycyl-glycine, Octadecenoic acid | | | | | | | |
| 25-Hydroxyvitamin D3, Citrulline, Glycyl-glycine | 3 | 0.76 | 0.65 | 0.87 | 0.79 | 0.63 | 0.96 |
| L-arginine, Etiocholanolone glucuronide | 2 | 0.75 | 0.63 | 0.88 | 0.76 | 0.56 | 0.96 |

TABLE 22
Exemplary Multivariable prediction for PE in the obese; stratum: black women; outcome: Total PE

| | | all · AUC | | | mean · AUC | | |
|---|---|---|---|---|---|---|---|
| | number of | | AUC lCI | AUC uCI | | AUC lCI | AUC uCI |
| predictors in model | predictors | AUC | 95% CI | 95% CI | AUC | 95% CI | 95% CI |
| Taurine, Stearoylcarnitine, Mat_hdl* | 3 | 0.87 | 0.80 | 0.93 | 0.85 | 0.72 | 0.97 |
| 3-Hydroxputanoic acid, Stearic acid | 2 | 0.77 | 0.65 | 0.88 | 0.76 | 0.55 | 0.91 |

TABLE 23
Exemplary Multivariable prediction for PE in the obese; stratum: non-black women; outcome: Total PE

| | | all · AUC | | | mean · AUC | | |
|---|---|---|---|---|---|---|---|
| | number of | | AUC lCI | AUC uCI | | AUC lCI | AUC uCI |
| predictors in model | predictors | AUC | 95% CI | 95% CI | AUC | 95% CI | 95% CI |
| Biliverdin, Etiocholanolone glucuronide, Met_XXX, Mat_sbp_m | 4 | 0.79 | 0.72 | 0.87 | 0.78 | 0.65 | 0.91 |
| NG-Monomethyl-L-arginine, Eicosapentaenoic acid, Biliverdin, | 4 | 0.79 | 0.73 | 0.85 | 0.78 | 0.66 | 0.89 |
| Etiocholanolone glucuronide* | | | | | | | |
| Glycyl-glycine, Biliverdin, Etiocholanolone glucuronide, PIGF | 4 | 0.79 | 0.73 | 0.85 | 0.79 | 0.68 | 0.90 |
| Dilinoleoyl-glycerol, Biliverdin, Etiocholanolone glucuronide, | 4 | 0.79 | 0.72 | 0.86 | 0.77 | 0.65 | 0.90 |
| Mat_sbp_m | | | | | | | |
| Hexadecanoic acid, NG-Monomethyl-L-arginine, Met_XXX, Mat_sbp_m | 4 | 0.79 | 0.72 | 0.86 | 0.77 | 0.65 | 0.89 |
| L-leucine, Glycyl-glycine, NG-Monomethyl-L-arginine, Biliverdin | 4 | 0.79 | 0.72 | 0.85 | 0.76 | 0.64 | 0.89 |
| NG-Monomethyl-L-arginine, Biliverdin, Etiocholanolone glucuronide, | 4 | 0.79 | 0.72 | 0.86 | 0.78 | 0.64 | 0.91 |
| Met_XXX | | | | | | | |
| Eicosapentaenoic acid, Biliverdin, Etiocholanolone glucuronide, PIGF | 4 | 0.79 | 0.72 | 0.85 | 0.78 | 0.66 | 0.89 |
| Biliverdin, Etiocholanolone glucuronide, Met_XXX, PIGF | 4 | 0.78 | 0.72 | 0.85 | 0.78 | 0.66 | 0.90 |
| Eicosapentaenoic acid, Biliverdin, Etiocholanolone glucuronide, | 4 | 0.78 | 0.72 | 0.84 | 0.78 | 0.67 | 0.89 |
| Mat_age_LT25 | | | | | | | |
| NG-Monomethyl-L-arginine, Biliverdin, Etiocholanolone glucuronide, PIGF | 4 | 0.78 | 0.72 | 0.84 | 0.77 | 0.66 | 0.88 |
| Biliverdin, Etiocholanolone glucuronide, Met_XXX, PIGF__MoM | 4 | 0.78 | 0.71 | 0.84 | 0.77 | 0.65 | 0.90 |
| Glycyl-glycine, Biliverdin, Mat_sbp_m | 3 | 0.78 | 0.71 | 0.85 | 0.78 | 0.66 | 0.90 |
| Linoleic acid, Biliverdin, Met_XXX, Mat_dbp_m | 4 | 0.78 | 0.71 | 0.85 | 0.76 | 0.62 | 0.89 |
| NG-Monomethyl-L-arginine, Biliverdin, Stearic acid, Met_XXX | 4 | 0.78 | 0.71 | 0.84 | 0.76 | 0.63 | 0.89 |
| Biliverdin, Etiocholanolone glucuronide, Mat_sbp_m | 3 | 0.78 | 0.70 | 0.85 | 0.76 | 0.63 | 0.88 |
| Octadecenoic acid, Biliverdin, Met_XXX, Mat_dbp_m | 4 | 0.77 | 0.71 | 0.84 | 0.76 | 0.64 | 0.89 |
| L-leucine, Glycyl-glycine, Met_XXX, Mat_sbp_m | 4 | 0.77 | 0.71 | 0.84 | 0.77 | 0.66 | 0.88 |
| Glycyl-glycine, L-Isoleucine, Met_XXX, Mat_sbp_m | 4 | 0.77 | 0.71 | 0.84 | 0.77 | 0.66 | 0.88 |
| L-arginine, Glycyl-glycine, NG-Monomethyl-L-arginine, Biliverdin | 4 | 0.77 | 0.70 | 0.84 | 0.75 | 0.62 | 0.88 |
| Glycyl-glycine, Biliverdin, Etiocholanolone glucuronide | 3 | 0.77 | 0.70 | 0.84 | 0.77 | 0.65 | 0.89 |
| 8, 11, 14 Eicosatrienoic acid, NG-Monomethyl-L-arginine, Biliverdin, | 4 | 0.77 | 0.70 | 0.84 | 0.76 | 0.64 | 0.89 |
| Met_XXX | | | | | | | |
| 8, 11, 14 Eicosatrienoic acid, Glycyl-glycine, Biliverdin, Met_XXX | 4 | 0.77 | 0.69 | 0.85 | 0.77 | 0.62 | 0.91 |
| Hexadecanoic acid, NG-Monomethyl-L-arginine, Biliverdin, Met_XXX | 4 | 0.77 | 0.69 | 0.84 | 0.77 | 0.64 | 0.89 |
| Glycyl-glycine, Linoleic acid, Biliverdin, Met_XXX | 4 | 0.77 | 0.69 | 0.84 | 0.75 | 0.61 | 0.89 |
| L-leucine, Dilinoleoyl-glycerol, NG-Monomethyl-L-arginine, Biliverdin | 4 | 0.77 | 0.70 | 0.83 | 0.75 | 0.64 | 0.86 |
| Glycyl-glycine, Hexadecanoic acid, Biliverdin, Met_XXX | 4 | 0.76 | 0.68 | 0.85 | 0.76 | 0.62 | 0.91 |
| NG-Monomethyl-L-arginine, Biliverdin, Etiocholanolone glucuronide | 3 | 0.76 | 0.70 | 0.83 | 0.75 | 0.64 | 0.86 |
| Glycyl-glycine, Eicosapentaenoic acid, Etiocholanolone glucuronide, | 4 | 0.76 | 0.70 | 0.83 | 0.76 | 0.64 | 0.88 |
| Mat_age_LT25 | | | | | | | |
| Hexadecanoic acid, Biliverdin, Met_XXX, PIGF | 4 | 0.76 | 0.69 | 0.83 | 0.76 | 0.63 | 0.88 |
| Glycyl-glycine, Octadecenoic acid, Biliverdin, Met_XXX | 4 | 0.76 | 0.68 | 0.84 | 0.75 | 0.61 | 0.89 |
| Octadecenoic acid, Biliverdin, Met_XXX, Mat_age_LT25 | 4 | 0.76 | 0.69 | 0.83 | 0.75 | 0.62 | 0.88 |
| Eicosapentaenoic acid, Biliverdin, Etiocholanolone glucuronide | 3 | 0.76 | 0.69 | 0.83 | 0.75 | 0.63 | 0.87 |
| L-leucine, Glycyl-glycine, Eicosapentaenoic acid, Etiocholanolone glucuronide | 4 | 0.76 | 0.69 | 0.83 | 0.75 | 0.63 | 0.88 |
| Glycyl-glycine, Met_XXX, Mat_sbp_m | 3 | 0.76 | 0.69 | 0.82 | 0.75 | 0.64 | 0.87 |
| L-leucine, Glycyl-glycine, Mat_sbp_m | 3 | 0.76 | 0.69 | 0.82 | 0.75 | 0.63 | 0.87 |
| Glycyl-glycine, Biliverdin, Mat_dbp_m | 3 | 0.75 | 0.68 | 0.83 | 0.75 | 0.62 | 0.89 |
| Biliverdin, Etiocholanolone glucuronide, Met_XXX | 3 | 0.75 | 0.68 | 0.83 | 0.75 | 0.61 | 0.89 |
| Biliverdin, Etiocholanolone glucuronide, PIGF | 3 | 0.75 | 0.68 | 0.82 | 0.75 | 0.63 | 0.87 |

TABLE 24
Exemplary Multivariable prediction for PE in the obese; stratum: Male Offspring; outcome: Total PE

| | | all · AUC | | | mean · AUC | | |
|---|---|---|---|---|---|---|---|
| | number of | | AUC lCI | AUC uCI | | AUC lCI | AUC uCI |
| predictors in model | predictors | AUC | 95% CI | 95% CI | AUC | 95% CI | 95% CI |
| Linoleic acid, Biliverdin, Met_XXX, Mat_sbp_m* | 4 | 0.81 | 0.72 | 0.90 | 0.77 | 0.58 | 0.96 |
| Biliverdin, Stearic acid, Met_XXX | 3 | 0.76 | 0.65 | 0.88 | 0.76 | 0.55 | 0.96 |
| Hexadecanoic acid, Biliverdin, Met_XXX | 3 | 0.76 | 0.63 | 0.88 | 0.76 | 0.54 | 0.97 |

TABLE 25
Exemplary Multivariable prediction for PE in the obese; stratum: Female Offspring; outcome: Total PE

| | | all · AUC | | | mean · AUC | | |
|---|---|---|---|---|---|---|---|
| | number of | | AUC lCI | AUC uCI | | AUC lCI | AUC uCI |
| predictors in model | predictors | AUC | 95% CI | 95% CI | AUC | 95% CI | 95% CI |
| L-leucine, NG-Monomethyl-L-arginine, Biliverdin, uncomp_pregn_score | 4 | 0.81 | 0.74 | 0.87 | 0.79 | 0.66 | 0.91 |
| L-leucine, Glycyl-glycine, Biliverdin, Mat_hdl | 4 | 0.79 | 0.71 | 0.86 | 0.79 | 0.67 | 0.91 |
| L-leucine, Glycyl-glycine, NG-Monomethyl-L-arginine* | 3 | 0.78 | 0.71 | 0.85 | 0.76 | 0.63 | 0.89 |
| L-leucine, Dilinoleoyl-glycerol, Glycyl-glycine, Stearoylcarnitine | 4 | 0.77 | 0.69 | 0.85 | 0.77 | 0.64 | 0.90 |
| L-arginine, NG-Monomethyl-L-arginine, Mat_hdl | 3 | 0.76 | 0.68 | 0.84 | 0.76 | 0.60 | 0.91 |
| Glycyl-glycine, L-Isoleucine, Biliverdin, Mat_hdl | 4 | 0.76 | 0.68 | 0.84 | 0.77 | 0.64 | 0.90 |
| L-arginine, Glycyl-glycine, Biliverdin, Mat_hdl | 4 | 0.76 | 0.67 | 0.85 | 0.77 | 0.62 | 0.91 |
| L-leucine, Glycyl-glycine, Biliverdin | 3 | 0.76 | 0.68 | 0.84 | 0.76 | 0.62 | 0.90 |

TABLE 26
Exemplary Multivariable prediction for PE in the obese; stratum: all; outcome: Term PE

| | | all · AUC | | | mean · AUC | | |
|---|---|---|---|---|---|---|---|
| | number of | | AUC lCI | AUC uCI | | AUC lCI | AUC uCI |
| predictors in model | predictors | AUC | 95% CI | 95% CI | AUC | 95% CI | 95% CI |
| L-leucine, Glycyl-glycine, NG-Monomethyl-L-arginine, Biliverdin* | 4 | 0.78 | 0.72 | 0.85 | 0.76 | 0.64 | 0.89 |
| Glycyl-glycine, Biliverdin, Etiocholanolone glucuronide, Mat_sbp_m | 4 | 0.78 | 0.71 | 0.85 | 0.78 | 0.66 | 0.90 |
| Dilinoleoyl-glycerol, NG-Monomethyl-L-arginine, Biliverdin, Mat_sbp_m | 4 | 0.78 | 0.72 | 0.84 | 0.77 | 0.65 | 0.88 |
| Biliverdin, Etiocholanolone glucuronide, Met_XXX, Mat_sbp_m | 4 | 0.77 | 0.70 | 0.85 | 0.76 | 0.61 | 0.90 |
| Glycyl-glycine, NG-Monomethyl-L-arginine, Asymmetric dimethylarginine, | 4 | 0.77 | 0.70 | 0.85 | 0.76 | 0.62 | 0.90 |
| Biliverdin | | | | | | | |
| L-leucine, NG-Monomethyl-L-arginine, Biliverdin, Etiocholanolone | 4 | 0.77 | 0.71 | 0.83 | 0.76 | 0.64 | 0.88 |
| glucuronide | | | | | | | |
| NG-Monomethyl-L-arginine, Biliverdin, Etiocholanolone glucuronide, | 4 | 0.77 | 0.70 | 0.83 | 0.76 | 0.63 | 0.88 |
| Mat_hdl | | | | | | | |
| 8, 11, 14 Eicosatrienoic acid, NG-Monomethyl-L-arginine, Biliverdin, | 4 | 0.77 | 0.69 | 0.84 | 0.75 | 0.62 | 0.88 |
| Met_XXX | | | | | | | |
| Hexadecanoic acid, NG-Monomethyl-L-arginine, Biliverdin, Met_XXX | 4 | 0.76 | 0.69 | 0.84 | 0.76 | 0.62 | 0.89 |
| Eicosapentaenoic acid, Biliverdin, Etiocholanolone glucuronide, | 4 | 0.76 | 0.70 | 0.83 | 0.75 | 0.63 | 0.88 |
| Mat_age_LT25 | | | | | | | |
| Glycyl-glycine, Biliverdin, Mat_sbp_m | 3 | 0.76 | 0.69 | 0.83 | 0.76 | 0.64 | 0.89 |
| Dilinoleoyl-glycerol, Eicosapentaenoic acid, Biliverdin, Etiocholanolone | 4 | 0.76 | 0.69 | 0.83 | 0.75 | 0.62 | 0.88 |
| glucuronide | | | | | | | |
| Glycyl-glycine, Biliverdin, Mat_dbp_m | 3 | 0.75 | 0.68 | 0.83 | 0.76 | 0.63 | 0.88 |
| Glycyl-glycine, Biliverdin, Etiocholanolone glucuronide | 3 | 0.75 | 0.68 | 0.82 | 0.75 | 0.63 | 0.87 |

TABLE 27
Exemplary Multivariable Rule-in AND/OR rule-out prediction for PE in the obese; stratum: all; outcome: Total PE

| | | all · AUC | | | | | | | | | |
|---|---|---|---|---|---|---|---|---|---|---|---|
| | | | AUC | AUC | | | | | | | |
| | number | | lCI | uCI | | | | | | | |
| | of | | 95% | 95% | Rule-out metrics | | Rule-in metrics | | n. | n. | select |
| predictors in model | predictors | AUC | CI | CI | sp@sn80 | sp@sn90 | sn@sp80 | sn@sp90 | controls | cases | improvement |
| NG-Monomethyl-L-arginine, | 2 | 0.72 | 0.66 | 0.79 | 0.55 | 0.24 | 0.51 | 0.37 | 633 | 63 | sp@sn80; |
| Mat_dbp_m | | | | | | | | | | | |
| Eicosapentaenoic acid, | 2 | 0.68 | 0.60 | 0.76 | 0.36 | 0.24 | 0.52 | 0.35 | 644 | 65 | sn@sp80; |
| Etiocholanolone glucuronide | | | | | | | | | | | |
| Etiocholanolone glucuronide, | 2 | 0.70 | 0.59 | 0.82 | 0.36 | 0.29 | 0.55 | 0.45 | 362 | 29 | sn@sp80; |
| Mat_prev_pet | | | | | | | | | | | |
| Decanoylcarnitine, Mat_prev_pet | 2 | 0.65 | 0.52 | 0.77 | 0.26 | 0.09 | 0.55 | 0.34 | 361 | 29 | sn@sp80; |
| Glycyl-glycine, Mat_prev_pet | 2 | 0.75 | 0.66 | 0.84 | 0.53 | 0.40 | 0.59 | 0.38 | 362 | 29 | sn@sp80; |
| | | | | | | | | | | | sp@sn80 |
| Dodecanoyl-l-carnitine, Mat_prev_pet | 2 | 0.66 | 0.54 | 0.78 | 0.24 | 0.14 | 0.55 | 0.38 | 362 | 29 | sn@sp80; |
| L-leucine, Dilinoleoyl-glycerol** | 2 | 0.66 | 0.60 | 0.72 | 0.51 | 0.31 | 0.32 | 0.23 | 645 | 65 | sp@sn80; |
| Met_XXX, Mat_dbp_m | 2 | 0.69 | 0.62 | 0.77 | 0.31 | 0.24 | 0.52 | 0.35 | 633 | 63 | sn@sp80; |
| NG-Monomethyl-L-arginine, | 2 | 0.72 | 0.65 | 0.79 | 0.50 | 0.25 | 0.55 | 0.37 | 645 | 65 | sp@sn80; |
| Asymmetric dimethylarginine | | | | | | | | | | | |
| Mat_prev_pet, PIGF | 2 | 0.67 | 0.55 | 0.79 | 0.38 | 0.12 | 0.52 | 0.34 | 362 | 29 | sn@sp80; |
| Glycyl-glycine, Mat_prev_pet, PIGF | 3 | 0.76 | 0.67 | 0.85 | 0.63 | 0.55 | 0.55 | 0.34 | 362 | 29 | sp@sn80; |
| Hexadecanoic acid, Mat_prev_pet, | 3 | 0.70 | 0.59 | 0.81 | 0.56 | 0.17 | 0.52 | 0.31 | 362 | 29 | sp@sn80; |
| PIGF | | | | | | | | | | | |
| Hexadecanoic acid, Biliverdin, | 3 | 0.72 | 0.64 | 0.79 | 0.37 | 0.27 | 0.58 | 0.51 | 643 | 65 | sn@sp80; |
| Met_XXX* | | | | | | | | | | | |
| Dilinoleoyl-glycerol, Bilirubin, | 3 | 0.72 | 0.62 | 0.82 | 0.38 | 0.35 | 0.59 | 0.38 | 362 | 29 | sn@sp80; |
| Mat_prev_pet | | | | | | | | | | | |
| Etiocholanolone glucuronide, Stearic | 3 | 0.73 | 0.62 | 0.85 | 0.58 | 0.21 | 0.59 | 0.55 | 362 | 29 | sp@sn80; |
| acid, Mat_prev_pet | | | | | | | | | | | |
| Glycyl-glycine, NG-Monomethyl-L- | 3 | 0.70 | 0.63 | 0.77 | 0.53 | 0.30 | 0.43 | 0.30 | 528 | 56 | sp@sn80; |
| arginine, uncomp_pregn_score | | | | | | | | | | | |

TABLE 28
Exemplary Multivariable Rule-in AND/OR rule-out prediction for PE in the obese; stratum: no GDM; outcome: Total PE

| | | all · AUC | | | | | | | | | |
|---|---|---|---|---|---|---|---|---|---|---|---|
| | number of | | AUC lCI | AUC uCI | Rule-out metrics | | Rule-in metrics | | | | select |
| predictors in model | predictors | AUC | 95% CI | 95% CI | sp@sn80 | sp@sn90 | sn@sp80 | sn@sp90 | n. controls | n. cases | improvement |
| NG-Monomethyl-L-arginine | 1 | 0.70 | 0.61 | 0.78 | 0.44 | 0.21 | 0.51 | 0.36 | 465 | 45 | sn@sp80; |
| Etiocholanolone glucuronide, Mat_prev_pet | 2 | 0.73 | 0.60 | 0.86 | 0.59 | 0.31 | 0.55 | 0.45 | 254 | 20 | sn@sp80; |
| Stearic acid, Met_XXX | 2 | 0.67 | 0.58 | 0.77 | 0.35 | 0.25 | 0.51 | 0.38 | 465 | 45 | sn@sp80; |
| Decanoylcarnitine, Mat_prev_pet | 2 | 0.68 | 0.52 | 0.84 | 0.32 | 0.10 | 0.65 | 0.45 | 253 | 20 | sn@sp80; |
| Glycyl-glycine, Mat_prev_pet | 2 | 0.73 | 0.61 | 0.85 | 0.52 | 0.39 | 0.55 | 0.35 | 254 | 20 | sn@sp80; sp@sn80 |
| NG-Monomethyl-L-arginine, Biliverdin | 2 | 0.74 | 0.67 | 0.81 | 0.61 | 0.35 | 0.47 | 0.29 | 463 | 45 | sp@sn80; |
| 8, 11, 14 Eicosatrienoic acid, Mat_prev_pet | 2 | 0.70 | 0.57 | 0.82 | 0.53 | 0.30 | 0.50 | 0.30 | 253 | 20 | sn@sp80; |
| 1-heptadecanoyl-2-hydroxy-sn-glycero-3-phosphocholine, Mat_prev_pet | 2 | 0.67 | 0.52 | 0.81 | 0.27 | 0.22 | 0.50 | 0.40 | 254 | 20 | sn@sp80; |
| Bilirubin, Mat_dbp_m | 2 | 0.71 | 0.63 | 0.80 | 0.44 | 0.28 | 0.53 | 0.42 | 458 | 43 | sn@sp80; |
| Dodecanoyl-l-carnitine, Mat_prev_pet | 2 | 0.67 | 0.52 | 0.83 | 0.25 | 0.12 | 0.65 | 0.40 | 254 | 20 | sn@sp80; |
| Stearoylcarnitine, Mat_prev_pet | 2 | 0.66 | 0.51 | 0.81 | 0.26 | 0.21 | 0.55 | 0.40 | 254 | 20 | sn@sp80; |
| L-leucine, Etiocholanolone glucuronide | 2 | 0.67 | 0.58 | 0.75 | 0.41 | 0.22 | 0.51 | 0.27 | 465 | 45 | sn@sp80; |
| Dilinoleoyl-glycerol, Mat_prev_pet | 2 | 0.79 | 0.69 | 0.88 | 0.70 | 0.46 | 0.55 | 0.40 | 254 | 20 | sn@sp80; sp@sn80 |
| L-leucine, Dilinoleoyl-glycerol | 2 | 0.69 | 0.62 | 0.76 | 0.50 | 0.29 | 0.40 | 0.24 | 465 | 45 | sp@sn80; |
| Choline, NG-Monomethyl-L-arginine | 2 | 0.74 | 0.66 | 0.82 | 0.55 | 0.35 | 0.51 | 0.40 | 465 | 45 | sp@sn80; |
| Met_XXX, Mat_dbp_m | 2 | 0.71 | 0.63 | 0.80 | 0.41 | 0.25 | 0.51 | 0.37 | 458 | 43 | sn@sp80; |
| Met_XXX, Mat_prev_pet | 2 | 0.70 | 0.55 | 0.85 | 0.26 | 0.13 | 0.55 | 0.40 | 254 | 20 | sn@sp80; |
| L-Isoleucine, NG-Monomethyl-L-arginine* | 2 | 0.74 | 0.65 | 0.83 | 0.47 | 0.23 | 0.64 | 0.44 | 465 | 45 | sn@sp80; |
| NG-Monomethyl-L-arginine, Mat_sbp_m** | 2 | 0.76 | 0.69 | 0.84 | 0.59 | 0.51 | 0.65 | 0.40 | 458 | 43 | sn@sp80; sp@sn80 |
| Mat_prev_pet, PIGF | 2 | 0.73 | 0.61 | 0.84 | 0.55 | 0.38 | 0.55 | 0.35 | 254 | 20 | sn@sp80; sp@sn80 |
| Linoleic acid, Stearoylcarnitine, Mat_prev_pet | 3 | 0.75 | 0.65 | 0.86 | 0.63 | 0.53 | 0.50 | 0.30 | 254 | 20 | sp@sn80; |
| Hexadecanoic acid, Bilirubin, Met_XXX | 3 | 0.70 | 0.60 | 0.80 | 0.29 | 0.17 | 0.60 | 0.47 | 465 | 45 | sn@sp80; |
| Hexadecanoic acid, Biliverdin, Met_XXX | 3 | 0.75 | 0.67 | 0.83 | 0.52 | 0.33 | 0.62 | 0.44 | 463 | 45 | sn@sp80; |
| Linoleic acid, Biliverdin, Met_XXX | 3 | 0.74 | 0.67 | 0.82 | 0.43 | 0.36 | 0.60 | 0.38 | 463 | 45 | sn@sp80; |
| Octadecenoic acid, Stearoylcarnitine, Mat_prev_pet | 3 | 0.72 | 0.61 | 0.84 | 0.56 | 0.30 | 0.45 | 0.35 | 254 | 20 | sp@sn80; |
| Eicosapentaenoic acid, Etiocholanolone glucuronide, | 3 | 0.75 | 0.61 | 0.89 | 0.58 | 0.27 | 0.70 | 0.50 | 253 | 20 | sn@sp80; |
| Mat_prev_pet | | | | | | | | | | | |
| L-Isoleucine, Met_XXX, PIGF | 3 | 0.69 | 0.61 | 0.78 | 0.53 | 0.13 | 0.56 | 0.24 | 465 | 45 | sp@sn80; |
| Linoleic acid, Met_XXX, PIGF | 3 | 0.71 | 0.63 | 0.79 | 0.56 | 0.28 | 0.47 | 0.29 | 465 | 45 | sp@sn80; |
| Octadecenoic acid, Biliverdin, Met_XXX | 3 | 0.74 | 0.66 | 0.82 | 0.48 | 0.37 | 0.56 | 0.47 | 463 | 45 | sn@sp80; |
| 8, 11, 14 Eicosatrienoic acid, Biliverdin, Met_XXX | 3 | 0.74 | 0.66 | 0.83 | 0.50 | 0.25 | 0.60 | 0.42 | 463 | 45 | sn@sp80; |
| Hexadecanoic acid, NG-Monomethyl-L-arginine, L- | 3 | 0.73 | 0.66 | 0.81 | 0.58 | 0.31 | 0.53 | 0.36 | 465 | 45 | sp@sn80; |
| Palmitoylcarnitine | | | | | | | | | | | |
| 3-Hydroxybutanoic acid, Linoleic acid, Mat_prev_pet | 3 | 0.74 | 0.62 | 0.85 | 0.57 | 0.49 | 0.55 | 0.30 | 254 | 20 | sn@sp80; |
| L-leucine, Biliverdin, Met_XXX | 3 | 0.71 | 0.62 | 0.79 | 0.52 | 0.19 | 0.53 | 0.27 | 463 | 45 | sn@sp80; |
| Choline, NG-Monomethyl-L-arginine, Mat_trig | 3 | 0.77 | 0.69 | 0.84 | 0.59 | 0.27 | 0.64 | 0.47 | 460 | 45 | sn@sp80; |
| L-leucine, Met_XXX, PIGF | 3 | 0.70 | 0.62 | 0.78 | 0.53 | 0.14 | 0.56 | 0.31 | 465 | 45 | sp@sn80; |
| Choline, NG-Monomethyl-L-arginine, Biliverdin | 3 | 0.77 | 0.69 | 0.84 | 0.58 | 0.35 | 0.64 | 0.29 | 463 | 45 | sn@sp80; |
| Linoleic acid, Eicosapentaenoic acid, Mat_prev_pet | 3 | 0.75 | 0.62 | 0.87 | 0.58 | 0.45 | 0.65 | 0.35 | 253 | 20 | sn@sp80; |
| Octadecenoic acid, Eicosapentaenoic acid, Mat_prev_pet | 3 | 0.70 | 0.58 | 0.83 | 0.60 | 0.47 | 0.40 | 0.35 | 253 | 20 | sp@sn80; |
| Stearoylcarnitine, Biliverdin, Met_XXX, 1-Palmitoyl-2-hydroxy- | 4 | 0.72 | 0.63 | 0.81 | 0.39 | 0.20 | 0.64 | 0.38 | 463 | 45 | sn@sp80; |
| sn-glycero-3-phosphocholine | | | | | | | | | | | |

TABLE 29
Exemplary Multivariable Rule-in AND/OR rule-out prediction for PE in the obese; stratum: GDM; outcome: Total PE*
*Note, the below selection of exemplary data is not comprehensive; the list has been truncated for presentation purposes.

| | | all · AUC | | | | | | | | | |
|---|---|---|---|---|---|---|---|---|---|---|---|
| | number of | | AUC lCI | AUC uCI | Rule-out metrics | | Rule-in metrics | | | | select |
| predictors in model | predictors | AUC | 95% CI | 95% CI | sp@sn80 | sp@sn90 | sn@sp80 | sn@sp90 | n. controls | n. cases | improvement |
| Mat_sbp_m | 1 | 0.76 | 0.64 | 0.88 | 0.55 | 0.34 | 0.61 | 0.33 | 164 | 18 | sn@sp80; sp@sn80 |
| Glycyl-glycine | 1 | 0.67 | 0.53 | 0.81 | 0.33 | 0.20 | 0.50 | 0.22 | 169 | 18 | sn@sp80; |
| 25-Hydroxyvitamin D3, Mat_dbp_m | 2 | 0.72 | 0.56 | 0.87 | 0.50 | 0.17 | 0.63 | 0.38 | 141 | 16 | sn@sp80; |
| Glycyl-glycine, Taurine | 2 | 0.75 | 0.66 | 0.85 | 0.56 | 0.52 | 0.50 | 0.33 | 169 | 18 | sp@sn80; |
| Citrulline, Taurine | 2 | 0.69 | 0.55 | 0.84 | 0.36 | 0.21 | 0.50 | 0.33 | 169 | 18 | sn@sp80; |
| Glycyl-glycine, L-Glutamine | 2 | 0.71 | 0.60 | 0.82 | 0.55 | 0.40 | 0.33 | 0.33 | 169 | 18 | sp@sn80; |
| Glycyl-glycine, Mat_bmi | 2 | 0.74 | 0.61 | 0.86 | 0.60 | 0.25 | 0.56 | 0.39 | 169 | 18 | sp@sn80; |
| Symmetric dimethylarginine, Taurine | 2 | 0.68 | 0.56 | 0.80 | 0.53 | 0.31 | 0.39 | 0.28 | 169 | 18 | sp@sn80; |
| Etiocholanolone glucuronide, Mat_dbp_m | 2 | 0.68 | 0.54 | 0.82 | 0.43 | 0.17 | 0.50 | 0.39 | 164 | 18 | sn@sp80; |
| Citrulline, Mat_bmi | 2 | 0.67 | 0.53 | 0.82 | 0.31 | 0.22 | 0.56 | 0.50 | 169 | 18 | sn@sp80; |
| Asymmetric dimethylarginine, Mat_sbp_m | 2 | 0.79 | 0.68 | 0.90 | 0.72 | 0.32 | 0.61 | 0.44 | 164 | 18 | sp@sn80; |
| 8, 11, 14 Eicosatrienoic acid, Met_XXX | 2 | 0.70 | 0.56 | 0.84 | 0.51 | 0.09 | 0.50 | 0.28 | 169 | 18 | sp@sn80; |
| Citrulline, Glycyl-glycine | 2 | 0.73 | 0.59 | 0.87 | 0.69 | 0.09 | 0.50 | 0.39 | 169 | 18 | sp@sn80; |
| Glycyl-glycine, Asymmetric dimethylarginine | 2 | 0.77 | 0.67 | 0.87 | 0.56 | 0.49 | 0.61 | 0.39 | 169 | 18 | sn@sp80; sp@sn80 |
| Citrulline, Asymmetric dimethylarginine | 2 | 0.71 | 0.58 | 0.83 | 0.41 | 0.26 | 0.61 | 0.33 | 169 | 18 | sn@sp80; |
| Taurine, Mat_sbp_m** | 2 | 0.83 | 0.73 | 0.92 | 0.71 | 0.62 | 0.72 | 0.39 | 164 | 18 | sp@sn80; |
| Asymmetric dimethylarginine, Biliverdin | 2 | 0.69 | 0.54 | 0.85 | 0.22 | 0.11 | 0.56 | 0.33 | 169 | 18 | sn@sp80; |
| Taurine, Mat_dbp_m | 2 | 0.74 | 0.63 | 0.84 | 0.59 | 0.40 | 0.44 | 0.22 | 164 | 18 | sp@sn80; |
| Glycyl-glycine, Biliverdin | 2 | 0.73 | 0.60 | 0.86 | 0.62 | 0.22 | 0.56 | 0.33 | 169 | 18 | sp@sn80; |
| Symmetric dimethylarginine, Met_XXX | 2 | 0.67 | 0.52 | 0.81 | 0.51 | 0.02 | 0.39 | 0.22 | 169 | 18 | sp@sn80; |
| Citrulline, Decanoylcarnitine, Etiocholanolone glucuronide | 3 | 0.73 | 0.61 | 0.86 | 0.56 | 0.27 | 0.61 | 0.28 | 169 | 18 | sn@sp80; |
| Asymmetric dimethylarginine, Biliverdin, L-Glutamine | 3 | 0.72 | 0.57 | 0.87 | 0.60 | 0.07 | 0.61 | 0.28 | 169 | 18 | sp@sn80; |
| 25-Hydroxyvitamin D3, 8, 11, 14 Eicosatrienoic acid, Mat_dbp_m | 3 | 0.73 | 0.60 | 0.86 | 0.62 | 0.30 | 0.50 | 0.31 | 141 | 16 | sp@sn80; |
| Citrulline, L-methionine, Taurine | 3 | 0.71 | 0.57 | 0.85 | 0.35 | 0.25 | 0.61 | 0.39 | 169 | 18 | sn@sp80; |
| 2-Hydroxybutanoic acid, L-leucine, Mat_bmi | 3 | 0.62 | 0.44 | 0.81 | 0.08 | 0.07 | 0.61 | 0.39 | 169 | 18 | sn@sp80; |
| Citrulline, Dodecanoyl-l-carnitine, Mat_sbp_m | 3 | 0.80 | 0.71 | 0.89 | 0.72 | 0.40 | 0.72 | 0.22 | 164 | 18 | sn@sp80; |
| 25-Hydroxyvitamin D3, Arachidonic acid, Mat_dbp_m | 3 | 0.73 | 0.59 | 0.87 | 0.61 | 0.29 | 0.44 | 0.38 | 141 | 16 | sp@sn80; |
| L-arginine, Glycyl-glycine, Sphingosine 1-phosphate | 3 | 0.76 | 0.65 | 0.86 | 0.66 | 0.48 | 0.44 | 0.39 | 169 | 18 | sp@sn80; |
| Citrulline, Glycyl-glycine, Linoleic acid | 3 | 0.73 | 0.59 | 0.87 | 0.47 | 0.19 | 0.67 | 0.28 | 169 | 18 | sn@sp80; |
| Citrulline, Glycyl-glycine, Taurine* | 3 | 0.78 | 0.66 | 0.90 | 0.64 | 0.32 | 0.67 | 0.50 | 169 | 18 | sn@sp80; |
| Citrulline, Decanoylcarnitine, Dodecanoyl-l-carnitine | 3 | 0.69 | 0.56 | 0.83 | 0.41 | 0.19 | 0.56 | 0.33 | 169 | 18 | sn@sp80; |
| Biliverdin, Stearic acid, Met_XXX | 3 | 0.64 | 0.48 | 0.80 | 0.34 | 0.22 | 0.50 | 0.33 | 169 | 18 | sn@sp80; |
| 25-Hydroxyvitamin D3, Glycyl-glycine, Sphingosine 1- | 4 | 0.78 | 0.67 | 0.89 | 0.71 | 0.39 | 0.69 | 0.38 | 146 | 16 | sn@sp80; |
| phosphate, Etiocholanolone glucuronide | | | | | | | | | | | |
| Citrulline, Docosahexaenoic acid, Glycyl-glycine, | 4 | 0.74 | 0.60 | 0.88 | 0.54 | 0.24 | 0.75 | 0.25 | 139 | 16 | sn@sp80; |
| uncomp_pregn_score | | | | | | | | | | | |
| Glycyl-glycine, Taurine, Biliverdin, Etiocholanolone glucuronide | 4 | 0.80 | 0.71 | 0.89 | 0.58 | 0.49 | 0.67 | 0.17 | 169 | 18 | sn@sp80; |
| 2-Hydroxybutanoic acid, Hexadecanoic acid, Taurine, | 4 | 0.70 | 0.55 | 0.85 | 0.45 | 0.12 | 0.63 | 0.31 | 139 | 16 | sn@sp80; |
| uncomp_pregn_score | | | | | | | | | | | |
| Decanoylcarnitine, Glycyl-glycine, Etiocholanolone glucuronide, Mat_hdl | 4 | 0.78 | 0.66 | 0.89 | 0.56 | 0.51 | 0.72 | 0.39 | 169 | 18 | sn@sp80; |
| Decanoylcarnitine, Linoleic acid, Taurine, Mat_hdl | 4 | 0.70 | 0.56 | 0.84 | 0.35 | 0.26 | 0.56 | 0.39 | 169 | 18 | sn@sp80; |
| Taurine, 1-heptadecanoyl-2-hydroxy-sn-glycero-3- | 4 | 0.71 | 0.55 | 0.87 | 0.35 | 0.21 | 0.69 | 0.31 | 139 | 16 | sn@sp80; |
| phosphocholine, PIGF, uncomp_pregn_score | | | | | | | | | | | |
| 8, 11, 14 Eicosatrienoic acid, Dodecanoyl-l-carnitine, Taurine, | 4 | 0.68 | 0.53 | 0.83 | 0.32 | 0.17 | 0.56 | 0.28 | 169 | 18 | sn@sp80; |
| Mat_hdl | | | | | | | | | | | |
| 2-Hydroxybutanoic acid, Asymmetric dimethylarginine, Stearic | 4 | 0.67 | 0.52 | 0.81 | 0.28 | 0.24 | 0.61 | 0.39 | 169 | 18 | sn@sp80; |
| acid, Met_XXX | | | | | | | | | | | |

TABLE 30
Exemplary Multivariable Rule-in AND/OR rule-out prediction for PE in the obese; stratum: bmi >= 40; outcome: Total PE*
*Note, the below selection of exemplary data is not comprehensive; the list has been truncated for presentation purposes.

| | | all · AUC | | | | | | | | | |
|---|---|---|---|---|---|---|---|---|---|---|---|
| | number of | | AUC lCI | AUC uCI | Rule-out metrics | | Rule-in metrics | | | | select |
| predictors in model | predictors | AUC | 95% CI | 95% CI | sp@sn80 | sp@sn90 | sn@sp80 | sn@sp90 | n. controls | n. cases | improvement |
| Bilirubin | 1 | 0.75 | 0.61 | 0.89 | 0.50 | 0.26 | 0.70 | 0.55 | 111 | 20 | sn@sp80; sp@sn80 |
| Citrulline, Biliverdin | 2 | 0.77 | 0.64 | 0.90 | 0.62 | 0.48 | 0.60 | 0.50 | 110 | 20 | sn@sp80; |
| Symmetric dimethylarginine, Biliverdin | 2 | 0.77 | 0.64 | 0.90 | 0.65 | 0.27 | 0.70 | 0.35 | 110 | 20 | sn@sp80; |
| Biliverdin, Mat_sbp_m | 2 | 0.80 | 0.68 | 0.92 | 0.49 | 0.40 | 0.68 | 0.53 | 105 | 19 | sn@sp80; |
| Biliverdin, Mat_dbp_m | 2 | 0.78 | 0.66 | 0.90 | 0.42 | 0.34 | 0.63 | 0.47 | 105 | 19 | sn@sp80; |
| Biliverdin, Mat_age_GE35 | 2 | 0.76 | 0.64 | 0.88 | 0.52 | 0.42 | 0.65 | 0.45 | 110 | 20 | sn@sp80; |
| Hexadecanoic acid, Biliverdin | 2 | 0.80 | 0.69 | 0.90 | 0.65 | 0.43 | 0.60 | 0.45 | 110 | 20 | sn@sp80; |
| Biliverdin, uncomp_pregn_score | 2 | 0.76 | 0.62 | 0.89 | 0.63 | 0.13 | 0.61 | 0.56 | 89 | 18 | sn@sp80; |
| Linoleic acid, Biliverdin | 2 | 0.77 | 0.67 | 0.88 | 0.55 | 0.48 | 0.55 | 0.40 | 110 | 20 | sn@sp80; |
| Taurine, Mat_sbp_m | 2 | 0.76 | 0.61 | 0.90 | 0.27 | 0.22 | 0.63 | 0.63 | 106 | 19 | sn@sp80; |
| Asymmetric dimethylarginine, Biliverdin | 2 | 0.77 | 0.66 | 0.88 | 0.71 | 0.58 | 0.60 | 0.40 | 110 | 20 | sn@sp80; |
| L-arginine, Biliverdin | 2 | 0.76 | 0.64 | 0.88 | 0.53 | 0.51 | 0.65 | 0.45 | 110 | 20 | sn@sp80; |
| Glycyl-glycine, Biliverdin | 2 | 0.79 | 0.68 | 0.90 | 0.68 | 0.49 | 0.70 | 0.25 | 110 | 20 | sn@sp80; |
| Octadecenoic acid, Biliverdin | 2 | 0.79 | 0.69 | 0.90 | 0.66 | 0.41 | 0.65 | 0.45 | 110 | 20 | sn@sp80; |
| 25-Hydroxyvitamin D3, L-arginine, Glycyl-glycine | 3 | 0.80 | 0.69 | 0.91 | 0.69 | 0.51 | 0.65 | 0.47 | 98 | 17 | sp@sn80; |
| Hexadecanoic acid, Biliverdin, Mat_sbp_m | 3 | 0.86 | 0.77 | 0.95 | 0.75 | 0.48 | 0.79 | 0.53 | 105 | 19 | sn@sp80; |
| Linoleic acid, Biliverdin, Mat_sbp_m* | 3 | 0.84 | 0.73 | 0.94 | 0.64 | 0.41 | 0.79 | 0.63 | 105 | 19 | sn@sp80; |
| 25-Hydroxyvitamin D3, Citrulline, Glycyl-glycine | 3 | 0.76 | 0.65 | 0.87 | 0.65 | 0.42 | 0.41 | 0.29 | 98 | 17 | sp@sn80; |
| Citrulline, Eicosapentaenoic acid, Stearic acid | 3 | 0.75 | 0.66 | 0.85 | 0.62 | 0.58 | 0.40 | 0.25 | 111 | 20 | sp@sn80; |
| Arachidonic acid, Hexadecanoic acid, Eicosapentaenoic acid | 3 | 0.75 | 0.64 | 0.87 | 0.63 | 0.45 | 0.65 | 0.35 | 111 | 20 | sn@sp80; |
| Hexadecanoic acid, Biliverdin, uncomp_pregn_score | 3 | 0.83 | 0.74 | 0.93 | 0.64 | 0.55 | 0.78 | 0.39 | 89 | 18 | sn@sp80; |
| Biliverdin, Myristic acid, Mat_sbp_m** | 3 | 0.86 | 0.77 | 0.95 | 0.77 | 0.60 | 0.74 | 0.63 | 105 | 19 | sn@sp80; sp@sn80 |
| 8, 11, 14 Eicosatrienoic acid, Biliverdin, Mat_sbp_m | 3 | 0.86 | 0.77 | 0.94 | 0.70 | 0.57 | 0.74 | 0.58 | 105 | 19 | sp@sn80; |
| Taurine, Eicosapentaenoic acid, Etiocholanolone glucuronide | 3 | 0.80 | 0.69 | 0.92 | 0.76 | 0.50 | 0.75 | 0.45 | 111 | 20 | sn@sp80; sp@sn80 |
| Arachidonic acid, Biliverdin, Mat_sbp_m | 3 | 0.85 | 0.76 | 0.93 | 0.75 | 0.47 | 0.68 | 0.47 | 105 | 19 | sp@sn80; |
| Docosahexaenoic acid, Taurine, Etiocholanolone glucuronide | 3 | 0.80 | 0.68 | 0.92 | 0.80 | 0.47 | 0.80 | 0.45 | 111 | 20 | sp@sn80; |
| Glycyl-glycine, Hexadecanoic acid, Biliverdin | 3 | 0.82 | 0.72 | 0.92 | 0.80 | 0.54 | 0.80 | 0.45 | 110 | 20 | sn@sp80; |
| Taurine, Stearoylcarnitine, Etiocholanolone glucuronide | 3 | 0.76 | 0.63 | 0.90 | 0.56 | 0.33 | 0.65 | 0.45 | 111 | 20 | sn@sp80; |
| 25-Hydroxyvitamin D3, L-arginine, Glycyl-glycine, Stearic acid | 4 | 0.82 | 0.72 | 0.92 | 0.72 | 0.56 | 0.76 | 0.41 | 98 | 17 | sn@sp80; |
| L-arginine, Docosahexaenoic acid, Stearoylcarnitine, Stearic acid | 4 | 0.78 | 0.68 | 0.87 | 0.68 | 0.68 | 0.55 | 0.25 | 111 | 20 | sn@sp80; |
| 25-Hydroxyvitamin D3, Citrulline, L-Palmitoylcarnitine, Myristic acid | 4 | 0.76 | 0.65 | 0.87 | 0.60 | 0.39 | 0.71 | 0.18 | 98 | 17 | sn@sp80; |
| 2-Hydroxybutanoic acid, L-arginine, Decanoylcarnitine, | 4 | 0.77 | 0.66 | 0.88 | 0.71 | 0.61 | 0.70 | 0.25 | 111 | 20 | sp@sn80; |
| Octadecenoic acid | | | | | | | | | | | |
| Taurine, Biliverdin, Etiocholanolone glucuronide, L-(+)- | 4 | 0.83 | 0.72 | 0.94 | 0.81 | 0.37 | 0.80 | 0.60 | 110 | 20 | sn@sp80; |
| Ergothioneine | | | | | | | | | | | |
| 25-Hydroxyvitamin D3, L-arginine, Glycyl-glycine, Mat_dbp_m | 4 | 0.79 | 0.67 | 0.91 | 0.78 | 0.44 | 0.75 | 0.38 | 93 | 16 | sn@sp80; |
| L-arginine, Eicosapentaenoic acid, Stearic acid, | 4 | 0.79 | 0.69 | 0.89 | 0.63 | 0.53 | 0.61 | 0.50 | 90 | 18 | sn@sp80; |
| uncomp_pregn_score | | | | | | | | | | | |
| 25-Hydroxyvitamin D3, 2-Hydroxybutanoic acid, Taurine, | 4 | 0.76 | 0.62 | 0.90 | 0.44 | 0.26 | 0.71 | 0.53 | 98 | 17 | sn@sp80; |
| Etiocholanolone glucuronide | | | | | | | | | | | |
| 25-Hydroxyvitamin D3, Citrulline, Taurine, Etiocholanolone | 4 | 0.80 | 0.67 | 0.92 | 0.64 | 0.38 | 0.76 | 0.41 | 98 | 17 | sn@sp80; |
| glucuronide | | | | | | | | | | | |
| 25-Hydroxyvitamin D3, L-leucine, Glycyl-glycine, | 4 | 0.79 | 0.67 | 0.90 | 0.64 | 0.37 | 0.56 | 0.56 | 81 | 16 | sp@sn80; |
| uncomp_pregn_score | | | | | | | | | | | |
| 25-Hydroxyvitamin D3, Glycyl-glycine, L-Isoleucine, | 4 | 0.78 | 0.68 | 0.88 | 0.63 | 0.47 | 0.53 | 0.35 | 98 | 17 | sp@sn80; |
| Etiocholanolone glucuronide | | | | | | | | | | | |
| L-arginine, Citrulline, Taurine, Etiocholanolone glucuronide | 4 | 0.80 | 0.68 | 0.92 | 0.78 | 0.37 | 0.75 | 0.45 | 111 | 20 | sn@sp80; |
| 25-Hydroxyvitamin D3, Arachidonic acid, Glycyl-glycine, | 4 | 0.86 | 0.77 | 0.94 | 0.80 | 0.56 | 0.82 | 0.41 | 97 | 17 | sp@sn80; |
| Biliverdin | | | | | | | | | | | |
| 2-Hydroxybutanoic acid, L-arginine, Taurine, Etiocholanolone | 4 | 0.79 | 0.67 | 0.92 | 0.62 | 0.27 | 0.75 | 0.60 | 111 | 20 | sn@sp80; |
| glucuronide | | | | | | | | | | | |
| 25-Hydroxyvitamin D3, L-leucine, Glycyl-glycine, | 4 | 0.77 | 0.67 | 0.88 | 0.65 | 0.48 | 0.53 | 0.35 | 98 | 17 | sp@sn80; |
| Etiocholanolone glucuronide | | | | | | | | | | | |
| 2-Hydroxybutanoic acid, L-arginine, Dilinoleoyl-glycerol, Stearic | 4 | 0.77 | 0.66 | 0.88 | 0.43 | 0.37 | 0.65 | 0.45 | 111 | 20 | sn@sp80; |
| acid | | | | | | | | | | | |
| L-arginine, Sphinganine-1-phosphate, Taurine, Mat_trig | 4 | 0.75 | 0.64 | 0.86 | 0.54 | 0.46 | 0.65 | 0.25 | 111 | 20 | sn@sp80; |

TABLE 31
Exemplary Multivariable Rule-in AND/OR rule-out prediction for PE in the obese; stratum: black; outcome: Total PE*
*Note, the below selection of exemplary data is not comprehensive; the list has been truncated for presentation purposes.

| | | all · AUC | | | | | | | | | |
|---|---|---|---|---|---|---|---|---|---|---|---|
| | number of | | AUC lCI | AUC uCI | Rule-out metrics | | Rule-in metrics | | | | select |
| predictors in model | predictors | AUC | 95% CI | 95% CI | sp@sn80 | sp@sn90 | sn@sp80 | sn@sp90 | n. controls | n. cases | improvement |
| Stearoylcarnitine, L-(+)-Ergothioneine | 2 | 0.76 | 0.63 | 0.90 | 0.52 | 0.52 | 0.58 | 0.33 | 132 | 12 | sn@sp80; |
| 3-Hydroxybutanoic acid, Stearic acid | 2 | 0.77 | 0.65 | 0.88 | 0.63 | 0.54 | 0.42 | 0.33 | 132 | 12 | sp@sn80; |
| Octadecenoic acid, Stearoylcarnitine | 2 | 0.77 | 0.65 | 0.88 | 0.59 | 0.50 | 0.50 | 0.33 | 132 | 12 | sp@sn80; |
| Stearoylcarnitine, Mat_trig | 2 | 0.78 | 0.63 | 0.93 | 0.58 | 0.33 | 0.67 | 0.58 | 132 | 12 | sn@sp80; |
| 3-Hydroxybutanoic acid, Mat_trig | 2 | 0.75 | 0.60 | 0.91 | 0.67 | 0.52 | 0.50 | 0.42 | 132 | 12 | sn@sp80; |
| 3-Hydroxybutanoic acid, Mat_hdl | 2 | 0.78 | 0.66 | 0.90 | 0.73 | 0.73 | 0.58 | 0.17 | 132 | 12 | sn@sp80; |
| Stearoylcarnitine, Myristic acid | 2 | 0.76 | 0.64 | 0.89 | 0.64 | 0.41 | 0.50 | 0.33 | 132 | 12 | sp@sn80; |
| Hexadecanoic acid, Stearoylcarnitine | 2 | 0.78 | 0.65 | 0.90 | 0.61 | 0.51 | 0.58 | 0.50 | 132 | 12 | sp@sn80; |
| L-(+)-Ergothioneine, Mat_hdl | 2 | 0.78 | 0.66 | 0.91 | 0.45 | 0.45 | 0.67 | 0.50 | 132 | 12 | sn@sp80; |
| 8, 11, 14 Eicosatrienoic acid, L-Acetylcarnitine, | 3 | 0.76 | 0.58 | 0.93 | 0.57 | 0.20 | 0.67 | 0.44 | 106 | 9 | sn@sp80; |
| uncomp_pregn_score | | | | | | | | | | | |
| 3-Hydroxybutanoic acid, Stearoylcarnitine, Myristic acid | 3 | 0.79 | 0.64 | 0.94 | 0.50 | 0.36 | 0.67 | 0.67 | 132 | 12 | sn@sp80; |
| 1-heptadecanoyl-2-hydroxy-sn-glycero-3-phosphocholine, | 3 | 0.76 | 0.58 | 0.95 | 0.80 | 0.22 | 0.83 | 0.42 | 132 | 12 | sn@sp80; |
| Bilirubin, Mat_hdl | | | | | | | | | | | |
| 3-Hydroxybutanoic acid, Hexadecanoic acid, Stearoylcarnitine* | 3 | 0.85 | 0.74 | 0.97 | 0.83 | 0.80 | 0.92 | 0.58 | 132 | 12 | sn@sp80; sp@sn80 |
| 3-Hydroxybutanoic acid, Stearoylcarnitine, Stearic acid | 3 | 0.86 | 0.77 | 0.95 | 0.77 | 0.61 | 0.75 | 0.50 | 132 | 12 | sn@sp80; |
| 8, 11, 14 Eicosatrienoic acid, Stearoylcarnitine, Mat_hdl | 3 | 0.83 | 0.74 | 0.92 | 0.70 | 0.69 | 0.67 | 0.33 | 132 | 12 | sn@sp80; |
| Linoleic acid, Stearoylcarnitine, PIGF | 3 | 0.76 | 0.61 | 0.91 | 0.47 | 0.46 | 0.67 | 0.42 | 132 | 12 | sn@sp80; |
| Taurine, Stearoylcarnitine, Mat_trig** | 3 | 0.84 | 0.73 | 0.95 | 0.71 | 0.51 | 0.75 | 0.50 | 132 | 12 | sp@sn80; |
| 2-Hydroxybutanoic acid, 8, 11, 14 Eicosatrienoic acid, | 3 | 0.77 | 0.61 | 0.93 | 0.52 | 0.35 | 0.67 | 0.50 | 132 | 12 | sn@sp80; |
| Stearoylcarnitine | | | | | | | | | | | |
| 3-Hydroxybutanoic acid, 8, 11, 14 Eicosatrienoic acid, | 3 | 0.82 | 0.71 | 0.93 | 0.77 | 0.61 | 0.75 | 0.33 | 132 | 12 | sn@sp80; |
| Stearoylcarnitine | | | | | | | | | | | |
| 3-Hydroxybutanoic acid, Met_XXX, Mat_hdl | 3 | 0.78 | 0.64 | 0.92 | 0.60 | 0.55 | 0.75 | 0.50 | 132 | 12 | sn@sp80; |
| Taurine, Stearoylcarnitine, Mat_hdl | 3 | 0.87 | 0.80 | 0.93 | 0.78 | 0.77 | 0.75 | 0.50 | 132 | 12 | sn@sp80; sp@sn80 |
| Homo-L-arginine, Stearoylcarnitine, Bilirubin, | 4 | 0.77 | 0.57 | 0.98 | 0.58 | 0.03 | 0.78 | 0.56 | 106 | 9 | sn@sp80; |
| uncomp_pregn_score | | | | | | | | | | | |
| Decanoylcarnitine, Hexadecanoic acid, NG-Monomethyl-L- | 4 | 0.82 | 0.71 | 0.93 | 0.70 | 0.51 | 0.56 | 0.56 | 106 | 9 | sp@sn80; |
| arginine, uncomp_pregn_score | | | | | | | | | | | |
| Dilinoleoyl-glycerol, Stearoylcarnitine, Bilirubin, | 4 | 0.77 | 0.54 | 0.99 | 0.39 | 0.00 | 0.78 | 0.67 | 106 | 9 | sn@sp80; |
| uncomp_pregn_score | | | | | | | | | | | |
| Glycyl-glycine, Stearoylcarnitine, Myristic acid, | 4 | 0.80 | 0.65 | 0.95 | 0.59 | 0.33 | 0.67 | 0.44 | 106 | 9 | sn@sp80; |
| uncomp_pregn_score | | | | | | | | | | | |
| Linoleic acid, NG-Monomethyl-L-arginine, L-Acetylcarnitine, | 4 | 0.82 | 0.65 | 0.99 | 0.73 | 0.21 | 0.67 | 0.56 | 106 | 9 | sn@sp80; |
| uncomp_pregn_score | | | | | | | | | | | |
| Decanoylcarnitine, Dilinoleoyl-glycerol, Stearoylcarnitine, Stearic acid | 4 | 0.78 | 0.64 | 0.92 | 0.48 | 0.45 | 0.67 | 0.42 | 132 | 12 | sn@sp80; |
| Taurine, Stearoylcarnitine, Myristic acid, Mat_trig | 4 | 0.86 | 0.77 | 0.95 | 0.82 | 0.73 | 0.83 | 0.50 | 132 | 12 | sp@sn80; |
| Choline, NG-Monomethyl-L-arginine, L-Lysine, | 4 | 0.82 | 0.62 | 1.00 | 0.81 | 0.04 | 0.89 | 0.44 | 106 | 9 | sn@sp80; |
| uncomp_pregn_score | | | | | | | | | | | |
| Hexadecanoic acid, NG-Monomethyl-L-arginine, | 4 | 0.85 | 0.72 | 0.98 | 0.73 | 0.42 | 0.78 | 0.56 | 106 | 9 | sn@sp80; |
| Stearoylcarnitine, uncomp_pregn_score | | | | | | | | | | | |

TABLE 32
Exemplary Multivariable Rule-in AND/OR rule-out prediction for PE in the obese; stratum: non-black; outcome: Total PE*
*Note, the below selection of exemplary data is not comprehensive; the list has been truncated for presentation purposes.

| | | all · AUC | | | | | | | | | |
|---|---|---|---|---|---|---|---|---|---|---|---|
| | number of | | AUC lCI | AUC uCI | Rule-out metrics | | Rule-in metrics | | | | select |
| predictors in model | predictors | AUC | 95% CI | 95% CI | sp@sn80 | sp@sn90 | sn@sp80 | sn@sp90 | n. controls | n. cases | improvement |
| NG-Monomethyl-L-arginine, Mat_dbp_m | 2 | 0.73 | 0.65 | 0.80 | 0.59 | 0.28 | 0.53 | 0.35 | 504 | 51 | sp@sn80; |
| Eicosapentaenoic acid, Etiocholanolone glucuronide | 2 | 0.71 | 0.63 | 0.80 | 0.40 | 0.25 | 0.57 | 0.34 | 512 | 53 | sn@sp80; |
| Biliverdin, Mat_sbp_m | 2 | 0.74 | 0.66 | 0.81 | 0.54 | 0.31 | 0.55 | 0.39 | 502 | 51 | sn@sp80; |
| Etiocholanolone glucuronide, Met_XXX | 2 | 0.68 | 0.59 | 0.77 | 0.41 | 0.14 | 0.51 | 0.36 | 513 | 53 | sn@sp80; |
| L-leucine, Dilinoleoyl-glycerol | 2 | 0.66 | 0.59 | 0.73 | 0.51 | 0.29 | 0.30 | 0.25 | 513 | 53 | sp@sn80; |
| Biliverdin, Etiocholanolone glucuronide | 2 | 0.73 | 0.66 | 0.80 | 0.48 | 0.38 | 0.51 | 0.36 | 511 | 53 | sn@sp80; |
| Met_XXX, Mat_dbp_m | 2 | 0.70 | 0.61 | 0.79 | 0.34 | 0.18 | 0.53 | 0.39 | 504 | 51 | sn@sp80; |
| L-leucine, Glycyl-glycine | 2 | 0.71 | 0.64 | 0.79 | 0.51 | 0.29 | 0.51 | 0.34 | 513 | 53 | sp@sn80; |
| NG-Monomethyl-L-arginine, Asymmetric dimethylarginine | 2 | 0.73 | 0.66 | 0.80 | 0.51 | 0.32 | 0.47 | 0.32 | 513 | 53 | sp@sn80; |
| NG-Monomethyl-L-arginine, Mat_sbp_m** | 2 | 0.77 | 0.70 | 0.84 | 0.64 | 0.42 | 0.61 | 0.35 | 504 | 51 | sp@sn80; |
| Met_XXX, Mat_trig | 2 | 0.65 | 0.55 | 0.74 | 0.28 | 0.05 | 0.55 | 0.34 | 508 | 53 | sn@sp80; |
| 25-Hydroxyvitamin D3, Linoleic acid, Met_XXX | 3 | 0.73 | 0.65 | 0.81 | 0.48 | 0.33 | 0.57 | 0.43 | 458 | 49 | sn@sp80; |
| Glycyl-glycine, Taurine, Met_XXX | 3 | 0.70 | 0.62 | 0.78 | 0.54 | 0.18 | 0.47 | 0.32 | 513 | 53 | sp@sn80; |
| Glycyl-glycine, Biliverdin, Etiocholanolone glucuronide | 3 | 0.77 | 0.70 | 0.84 | 0.61 | 0.51 | 0.55 | 0.38 | 511 | 53 | sp@sn80; |
| L-arginine, Glycyl-glycine, NG-Monomethyl-L-arginine | 3 | 0.76 | 0.69 | 0.82 | 0.67 | 0.54 | 0.53 | 0.30 | 513 | 53 | sp@sn80; |
| Hexadecanoic acid, Biliverdin, Met_XXX | 3 | 0.74 | 0.66 | 0.82 | 0.44 | 0.26 | 0.60 | 0.53 | 511 | 53 | sn@sp80; |
| Linoleic acid, Biliverdin, Met_XXX | 3 | 0.74 | 0.67 | 0.82 | 0.40 | 0.31 | 0.62 | 0.40 | 511 | 53 | sn@sp80; |
| Octadecenoic acid, Biliverdin, Met_XXX | 3 | 0.74 | 0.66 | 0.82 | 0.41 | 0.34 | 0.58 | 0.51 | 511 | 53 | sn@sp80; |
| 8, 11, 14 Eicosatrienoic acid, Biliverdin, Met_XXX | 3 | 0.74 | 0.66 | 0.82 | 0.41 | 0.27 | 0.60 | 0.47 | 511 | 53 | sn@sp80; |
| 25-Hydroxyvitamin D3, Stearic acid, Met_XXX | 3 | 0.73 | 0.65 | 0.81 | 0.60 | 0.24 | 0.53 | 0.41 | 458 | 49 | sp@sn80; |
| Docosahexaenoic acid, Biliverdin, Met_XXX | 3 | 0.71 | 0.63 | 0.79 | 0.35 | 0.23 | 0.60 | 0.38 | 511 | 53 | sn@sp80; |
| Hexadecanoic acid, Biliverdin, PIGF | 3 | 0.71 | 0.64 | 0.78 | 0.50 | 0.33 | 0.53 | 0.28 | 511 | 53 | sn@sp80; |
| Biliverdin, Stearic acid, Met_XXX* | 3 | 0.74 | 0.67 | 0.82 | 0.49 | 0.31 | 0.62 | 0.43 | 511 | 53 | sn@sp80; |
| 8, 11, 14 Eicosatrienoic acid, Dilinoleoyl-glycerol, Eicosapentaenoic acid, Biliverdin | 4 | 0.73 | 0.66 | 0.79 | 0.61 | 0.38 | 0.45 | 0.23 | 511 | 53 | sp@sn80; |
| Dilinoleoyl-glycerol, Glycyl-glycine, L-Isoleucine, Urea | 4 | 0.72 | 0.65 | 0.79 | 0.56 | 0.27 | 0.53 | 0.34 | 513 | 53 | sp@sn80; |
| 25-Hydroxyvitamin D3, Glycyl-glycine, Biliverdin, | 4 | 0.78 | 0.71 | 0.86 | 0.58 | 0.44 | 0.65 | 0.49 | 457 | 49 | sn@sp80; |
| Etiocholanolone glucuronide | | | | | | | | | | | |
| 25-Hydroxyvitamin D3, Linoleic acid, Biliverdin, L-(+)-Ergothioneine | 4 | 0.72 | 0.64 | 0.80 | 0.45 | 0.26 | 0.59 | 0.35 | 457 | 49 | sn@sp80; |
| Arachidonic acid, Eicosapentaenoic acid, Met_XXX, Mat_age_LT25 | 4 | 0.68 | 0.59 | 0.76 | 0.34 | 0.25 | 0.55 | 0.32 | 512 | 53 | sn@sp80; |

TABLE 32
Exemplary Multivariable Rule-in AND/OR rule-out prediction for PE in the obese; stratum: all; outcome: Term PE*
*Note, the below selection of exemplary data is not comprehensive; the list has been truncated for presentation purposes.

| | | all · AUC | | | | | | | | | |
|---|---|---|---|---|---|---|---|---|---|---|---|
| | number of | | AUC lCI | AUC uCI | Rule-out metrics | | Rule-in metrics | | | | select |
| predictors in model | predictors | AUC | 95% CI | 95% CI | sp@sn80 | sp@sn90 | sn@sp80 | sn@sp90 | n. controls | n. cases | improvement |
| Hexadecanoic acid, Eicosapentaenoic acid | 2 | 0.69 | 0.63 | 0.75 | 0.50 | 0.42 | 0.37 | 0.15 | 644 | 52 | sp@sn80; |
| NG-Monomethyl-L-arginine, Mat_dbp_m | 2 | 0.73 | 0.66 | 0.80 | 0.60 | 0.33 | 0.48 | 0.38 | 633 | 50 | sp@sn80; |
| Eicosapentaenoic acid, Etiocholanolone glucuronide | 2 | 0.68 | 0.59 | 0.76 | 0.33 | 0.24 | 0.54 | 0.37 | 644 | 52 | sn@sp80; |
| 25-Hydroxyvitamin D3, 1-Palmitoyl-2-hydroxy-sn-glycero-3-phosphocholine | 2 | 0.66 | 0.57 | 0.75 | 0.33 | 0.18 | 0.50 | 0.33 | 568 | 48 | sn@sp80; |
| NG-Monomethyl-L-arginine, Biliverdin | 2 | 0.71 | 0.64 | 0.79 | 0.54 | 0.32 | 0.44 | 0.27 | 643 | 52 | sp@sn80; |
| Biliverdin, Mat_sbp_m | 2 | 0.72 | 0.64 | 0.79 | 0.40 | 0.29 | 0.52 | 0.40 | 631 | 50 | sn@sp80; |
| Etiocholanolone glucuronide, Met_XXX | 2 | 0.65 | 0.55 | 0.75 | 0.16 | 0.09 | 0.52 | 0.42 | 645 | 52 | sn@sp80; |
| Met_XXX, Mat_bmi | 2 | 0.65 | 0.56 | 0.74 | 0.30 | 0.13 | 0.56 | 0.38 | 645 | 52 | sn@sp80; |
| Hexadecanoic acid, Biliverdin | 2 | 0.70 | 0.63 | 0.77 | 0.53 | 0.38 | 0.42 | 0.27 | 643 | 52 | sp@sn80; |
| L-(+)-Ergothioneine, Mat_dbp_m | 2 | 0.68 | 0.59 | 0.76 | 0.42 | 0.24 | 0.52 | 0.30 | 633 | 50 | sn@sp80; |
| L-leucine, Dilinoleoyl-glycerol | 2 | 0.66 | 0.60 | 0.73 | 0.50 | 0.35 | 0.31 | 0.17 | 645 | 52 | sp@sn80; |
| Met_XXX, Mat_dbp_m | 2 | 0.68 | 0.60 | 0.77 | 0.29 | 0.24 | 0.52 | 0.32 | 633 | 50 | sn@sp80; |
| NG-Monomethyl-L-arginine, Mat_sbp_m | 2 | 0.74 | 0.67 | 0.81 | 0.61 | 0.43 | 0.54 | 0.34 | 633 | 50 | sp@sn80; |
| Hexadecanoic acid, Urea, Eicosapentaenoic acid | 3 | 0.71 | 0.63 | 0.78 | 0.48 | 0.40 | 0.52 | 0.33 | 644 | 52 | sn@sp80; |
| Hexadecanoic acid, Bilirubin, Met_XXX | 3 | 0.71 | 0.62 | 0.80 | 0.31 | 0.19 | 0.60 | 0.42 | 645 | 52 | sn@sp80; |
| Hexadecanoic acid, Biliverdin, Met_XXX* | 3 | 0.74 | 0.66 | 0.82 | 0.40 | 0.34 | 0.62 | 0.54 | 643 | 52 | sn@sp80; |
| 25-Hydroxyvitamin D3, Myristic acid, Met_XXX | 3 | 0.69 | 0.61 | 0.78 | 0.37 | 0.20 | 0.54 | 0.35 | 568 | 48 | sn@sp80; |
| 8, 11, 14 Eicosatrienoic acid, Urea, Eicosapentaenoic acid | 3 | 0.70 | 0.62 | 0.77 | 0.44 | 0.27 | 0.54 | 0.31 | 644 | 52 | sn@sp80; |
| 25-Hydroxyvitamin D3, Glycyl-glycine, Etiocholanolone glucuronide | 3 | 0.72 | 0.63 | 0.80 | 0.38 | 0.26 | 0.58 | 0.42 | 568 | 48 | sn@sp80; |
| Choline, Glycyl-glycine, NG-Monomethyl-L-arginine | 3 | 0.72 | 0.64 | 0.80 | 0.47 | 0.19 | 0.60 | 0.33 | 645 | 52 | sn@sp80; |
| L-arginine, Glycyl-glycine, NG-Monomethyl-L-arginine, Biliverdin** | 4 | 0.77 | 0.70 | 0.84 | 0.68 | 0.40 | 0.63 | 0.40 | 643 | 52 | sp@sn80; |

### REFERENCES

1. Nanda, S., Savvidou, M., Syngelaki, A., Akolekar, R. & Nicolaides, K. H. Prediction of gestational diabetes mellitus by maternal factors and biomarkers at 11 to 13 weeks. Prenat. Diagn. 31, 135-41 (2011).
2. Gabbay-Benziv, R., Doyle, L. E., Blitzer, M. & Baschat, A. A. First trimester prediction of maternal glycemic status. J. Perinat. Med. 43, 283-289 (2015).
3. TEEDE, H. J., HARRISON, C. L., TEH, W. T., PAUL, E. & ALLAN, C. A. Gestational diabetes: Development of an early risk prediction tool to facilitate opportunities for prevention. Aust. New Zeal. J. Obstet. Gynaecol. 51, 499-504 (2011).
4. Van Leeuwen, M. et al. Estimating the risk of gestational diabetes mellitus: A clinical prediction model based on patient characteristics and medical history. BJOG An Int. J. Obstet. Gynaecol. 117, 69-75 (2010).
5. Emerson, D. S., Felker, R. E. & Brown, D. L. The sagittal sign. An early second trimester sonographic indicator of fetal gender. J. Ultrasound Med. 8, 293-297 (1989).
6. Efrat, Z., Akinfenwa, O. O. & Nicolaides, K. H. First-trimester determination of fetal gender by ultrasound. Ultrasound Obstet. Gynecol. 13, 305-307 (1999).
7. Alfirevic, Z., Mujezinovic, F. & Sundberg, K. in Cochrane Database of Systematic Reviews (ed. Alfirevic, Z.) 148-156 (John Wiley & Sons, Ltd, 2003). doi: 10.1002/14651858.CD003252
8. Hackett, G. A. et al. Early amniocentesis at 11-14 weeks' gestation for the diagnosis of fetal chromosomal abnormality—a clinical evaluation. Prenat. Diagn. 11, 311-315 (1991).
9. Mazloom, A. R. et al. Noninvasive prenatal detection of sex chromosomal aneuploidies by sequencing circulating cell-free DNA from maternal plasma. Prenat. Diagn. 33, 591-597 (2013).
10. Costa, J. M. et al. First-trimester fetal sex determination in maternal serum using real-time PCR. Prenat. Diagn. 21, 1070-4 (2001).

What is claimed herein:

1. A method comprising:
determining the level of at least one of:
Bilirubin, Citrulline, 2-Hydroxybutanoic acid, Dodecanoyl-I-carnitine, Hexadecanoic acid, Myristic acid, Linoleic acid, Arachidonic acid, 8,11,14 Eicosatrienoic acid, Docosahexaenoic acid, Octadecenoic acid, and Stearic acid;
in a blood sample obtained from a pregnant obese woman with a BMI of 40 or greater.

2. The method of claim 1, comprising determining the level of at least one of:
2-Hydroxybutanoic acid, Dodecanoyl-I-carnitine, Hexadecanoic acid, Myristic acid, Linoleic acid, Arachidonic acid, 8,11,14 Eicosatrienoic acid, Docosahexaenoic acid, Octadecenoic acid, and Stearic acid.

3. The method of claim 1, comprising determining the level of at least one of:
Bilirubin and Citrulline.

4. The method of claim 1, further comprising determining the level of an obese pregnancy specific protein selected from:
Placental Growth Factor (PIGF), soluble endoglin, and Pregnancy-Associated Plasma Protein A (PAPPA).

5. A method comprising:
determining the level of at least one of:
Stearoylcarnitine, 3-Hydroxybutanoic acid and Stearic acid;
in a blood sample obtained from a pregnant obese woman of black ethnicity.

6. The method of claim 5, comprising determining the level of Stearoylcarnitine.

7. The method of claim 5, comprising determining the level of 3-Hydroxybutanoic acid.

8. The method of claim 5, comprising determining the level of Stearic acid.

9. The method of claim 5, comprising determining the level of Stearoylcarnitine and 3-Hydroxybutanoic acid.

10. The method of claim 5, comprising determining the level of Stearoylcarnitine acid Stearic acid.

11. The method of claim 5, comprising determining the level of 3-Hydroxybutanoic acid and Stearic acid.

12. The method of claim 5, further comprising determining the level of an obese pregnancy specific protein selected from:
Placental Growth Factor (PIGF), soluble endoglin, and Pregnancy-Associated Plasma Protein A (PAPPA).

13. A method comprising:
determining the level of at least one of:
I-leucine, I-isoleucine, Hexadecanoic acid, Octadecenoic acid, Linoleic acid, Docosahexaenoic acid, and Eicosapentaenoic acid;
in a blood sample obtained from a pregnant obese woman of non-black ethnicity.

14. The method of claim 13, comprising determining the level of at least one of:
Hexadecanoic acid, Octadecenoic acid, Linoleic acid, Docosahexaenoic acid, and Eicosapentaenoic acid.

15. The method of claim 13, comprising determining the level of at least one of I-leucine and L-isoleucine.

16. The method of claim 13, further comprising determining the level of an obese pregnancy specific protein selected from:
Placental Growth Factor (PIGF), soluble endoglin, and Pregnancy-Associated Plasma Protein A (PAPPA).
